(19) United States
(12) Patent Application Publication
(10) Pub. No.: US 2024/0335474 A1
(43) Pub. Date: Oct. 10, 2024

(54) COMPOSITIONS AND METHODS FOR IMMUNOTHERAPY

(71) Applicants: Gracell Biotechnologies (Shanghai) Co., Ltd., Shanghai (CN); Suzhou Gracell Biotechnologies Co., Ltd., Jiangsu (CN)

(72) Inventors: Jiaping HE, Shanghai (CN); Zhe SUN, Shanghai (CN); Yongliang ZHANG, Shanghai (CN); Nanjing LIN, Shanghai (CN); Yan HE, Shanghai (CN); Xin LIU, Shanghai (CN); Chao LI, Shanghai (CN); Jinghua LIU, Shanghai (CN); Lianjun SHEN, Shanghai (CN); Pengfei JIANG, Shanghai (CN); Wei CAO, Shanghai (CN); Liping LIU, Shanghai (CN)

(21) Appl. No.: 18/745,930

(22) Filed: Jun. 17, 2024

Related U.S. Application Data
(63) Continuation of application No. 17/338,972, filed on Jun. 4, 2021.
Application No. 17/338,972 is a continuation of application No. PCT/CN2019/123684, filed on Dec. 6, 2019.

(30) Foreign Application Priority Data
Dec. 7, 2018 (CN) 201811501797.8
Jan. 25, 2019 (WO) PCT/CN2019/073252
Apr. 12, 2019 (CN) 201910297171.8
Jul. 26, 2019 (WO) PCT/CN2019/097996

(51) Int. Cl. A61K 35/17; A61K 38/00; C07K 14/725

(52) U.S. Cl. CPC A61K 35/17; C07K 14/7051; A61K 38/00; C07K 2319/02; C07K 2319/03

(57) ABSTRACT

The present disclosure provides compositions and methods for engineered cellular compositions and methods of immunotherapy utilizing the same. Compositions of the present disclosure for immune cell regulation comprise a chimeric antigen receptor polypeptide, a T cell receptor polypeptide, and combinations thereof.

### CROSS-REFERENCE

This application is a continuation of International Application No. PCT/CN2019/073252, filed Jan. 25, 2019; and International Application No. PCT/CN2019/097996, filed Jul. 26, 2019; and PCT Application No. PCT/CN2019/123684, filed Dec. 6, 2019, which claim priority to Chinese Patent Application No. 201811501797.8, filed Dec. 7, 2018 and Chinese Patent Application No. 201910297171.8, filed Apr. 12, 2019; each of which is entirely incorporated herein by reference.

### SEQUENCE LISTING

The instant application contains a Sequence Listing which has been submitted electronically in ASCII format and is hereby incorporated by reference in its entirety. Said ASCII copy, created on Jan. 9, 2020, is named 181910172FF_SL.txt and is 14,481 bytes in size.

### BACKGROUND

Adoptive T cell therapy, as an example of immunotherapy, involves the isolation and ex vivo expansion of tumor specific T cells to achieve greater number of T cells than what could be obtained by vaccination alone. The tumor specific T cells are then infused into patients with cancer to provide their immune systems the ability to overwhelm remaining tumor via T cells which can attack and kill the cancer. While there are many forms of adoptive T cell therapy for cancer, they by and large suffer from various deficiencies. Amongst them are cellular exhaustion, long preparation time, and ineffective compositions of engineered cells.

### SUMMARY

In view of the foregoing, there exists a considerable need for alternative compositions and methods to carry out immunotherapy. The compositions and methods of the present disclosure address this need, and provide additional advantages as well. In particular, the various aspects of the disclosure provide systems for immune cell regulation.

In an aspect, the present disclosure provides a method of administering a cell therapy comprising engineered immune cells expressing a chimeric antigen receptor (CAR) and/or an engineered T cell receptor (TCR), comprising: infusing a population of immune cells comprising the engineered immune cells into a subject in need thereof, wherein the engineered immune cells have not been subject to ex vivo expansion for no more than 2 weeks or 1 week, and wherein the population is further characterized in that: central memory T cells (TCM) in the population are more abundant than effector memory T cells (TEM). In some embodiments, the engineered immune cells have been subject to ex vivo expansion less than 13, 12, 10, 9, 8, 7, 6, 5, 4, 3, 2, or 1 days. In some embodiments, the engineered immune cells have been subject to ex vivo expansion less than 6, 5, 4, 3, 2, or 1 days. In some embodiments, the engineered immune cells have been subject to ex vivo expansion less than 5 days. In some embodiments, the engineered immune cells have been subject to ex vivo expansion less than 72, 48, 36, 32, or 24 hours. In some embodiments, the TCM are CD45RO⁺CD62L⁺. In some embodiments, the TEM are CD45RO⁺CD62L−. In some embodiments, a population is further characterized in that it is less abundant in PD1 and LAG3. In some embodiments, reduced exhaustion of cells in a population is observed as compared to the exhaustion of cells in a comparable population that undergoes an ex vivo expansion for no more than 2 weeks or 1 week, or 10 or 9 days.

In some aspects, reduced exhaustion of a population is characterized in that the population comprises less cells expressing PD1 and LAG3.

In some embodiments, the engineered immune cells are T cells, NK cells, and/or NKT cells. In some embodiments, the TCR comprises (i) a ligand binding domain specific for a ligand and (ii) a transmembrane domain. In some embodiments, the CAR comprises: (i) a ligand binding domain specific for a ligand, (ii) a transmembrane domain, and (iii) an intracellular signaling domain. In some embodiments, the ligand of the TCR or CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments the transmembrane domain is from CD8a, CD4, CD28, CD45, PD-1 and/or CD152. In some embodiments, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least 2 intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the CAR further comprises a signal peptide, and wherein the signal peptide is derived from IgG1, GM-CSF and/or CD8α. In some embodiments, the engineered immune cells are from peripheral blood, cord blood, bone marrow, and/or induced pluripotent stem cells. In some embodiments, the engineered immune cells are from peripheral blood, and wherein the peripheral blood cells are T cells. In some embodiments, greater memory and/or stemness is observed in a population as compared a comparable population that undergoes an ex vivo expansion for no more than 2 weeks or 1 week. In some embodiments, there are 2 fold more TCM as compared to TEM. In some embodiments, there are 4 fold more TCM as compared to TEM. In some embodiments, the infusing is intravenous. In some embodiments, the administering comprises infusing from about 1×10⁴/kg body weight of engineered immune cells. In some embodiments, the administering comprises infusing from about 3×10⁵/kg body weight of engineered immune cells. In some embodiments, at least 10% of the immune cells express the CAR and/or the TCR. In some embodiments, at least 20% of the immune cells express the CAR and/or the TCR. In some embodiments, at least 40% of the immune cells express the CAR and/or the TCR. In some embodiments, a method further comprises administering a secondary agent to the subject in need thereof. In some embodiments, the secondary agent is a therapeutically effective amount of an immunostimulant, immunosuppressive, anti-fungal, antibiotic, anti-angiogenic, chemotherapeutic, radioactive, and/or an antiviral. In some aspects, the immunostimulant is IL-2. In some aspects, a method further comprises obtaining peripheral blood from the subject in need thereof after the administering. In some aspects, the engineered immune cells in the subject are quantified from the peripheral blood. In some embodiments, a level of a growth factor in the subject is quantified. In some embodiments, the growth factor is selected from the group consisting of IL-10, IL-6, tumor necrosis factor α (TNF-α), IL-1β, IL-2, IL-4, IL-8, IL-12, and/or IFN-γ. In some aspects, a method comprises repeating the infusing. In some embodiments, the population of immune cells is allogeneic to the subject in need thereof. In some aspects, the population of immune cells is autologous to the subject in need thereof. In some aspects, the subject has cancer. In some embodiments, the cancer is hematological. In some aspects, the hematological cancer is leukemia, myeloma, lymphoma, and/or a combination thereof. In some embodiments, the leukemia is chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL). In some embodiments, the lymphoma is mantle cell lymphoma (MCL), T cell lymphoma, Hodgkin's lymphoma, and/or non-Hodgkin's lymphoma. In some embodiments, the cancer is solid. In some embodiments, the solid cancer is selected from the group comprising: nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, or bladder cancer. In some embodiments, the subject in need thereof has a BCR-ABL mutation, and the mutation is in a BCR-ABL kinase domain. In some embodiments, the subject in need thereof has a T315I and/or V299L mutation in the BCR-ABL kinase domain. In some aspects, the subject shows resistance to a tyrosine kinase inhibitor. In some aspects, the subject has a tumor or is susceptible of having a tumor after chemotherapy. In some aspects, the subject was pre-treated with chemotherapy prior to the administration. In some aspects, the chemotherapy comprises an administration of fludarabine, cyclophosphamide and/or cytarabine. In some embodiments, the subject has minimal residual disease (MRD), and the MRD is acute lymphoblastic leukemia.

In some embodiments, the subject population of immune cells is further characterized in that a greater proliferation, cytotoxicity, and/or bone marrow migration is observed in the population as compared to the proliferation, cytotoxicity, and/or bone marrow migration of a comparable population that undergoes an ex vivo expansion for no more than 2 weeks or 1 week, for example C-CART. In some embodiments, a subject population of cells, such as F-CART, can be evaluated using an assay that determines a level of: migration, proliferation, cytotoxicity and effector activity. A level of migration can be determined using a chemotaxis assay. In an aspect, migration can refer to migration into the bone marrow. In an aspect, migration can refer to migration out of the bone marrow. In an aspect, migration can also refer to a movement towards a target, for example a chemokine or a cancer cell. In an aspect, a chemokine system includes more than 40 chemokines and more than 18 chemokine receptors. Chemokine receptors are defined by their ability to induce directional migration of cells, such as engineered immune cells, toward a gradient of a chemotactic cytokine (chemotaxis). Chemokine receptors are a family of 7 transmembrane domain, G-protein-coupled cell surface receptors that are designated CXCR1 through CXCR5, CCR1 through CCR11, XCR1, and CX3CR1, based on their specific preference for certain chemokines. Chemokines are small secreted proteins that can be segregated into 2 main subfamilies based on whether the 2 conserved cysteine residues present in all chemokines are separated by an intervening amino acid, respectively accounting for CXC or CC chemokines. In some embodiments, migration or chemotaxis can be quantified in a population of engineered immune cells. In an aspect, migration or chemotaxis to a cancer can be evaluated in vitro using the CXCR4 and ligand SDF-1 (CXCL12) axis. In an aspect, a greater percent of CD3, CD4, and/or CD8 F-CART that express CXCR4 as compared to CD3, CD4, and/or CD8 C-CART that express CXCR4. In an aspect, a greater mean fluorescent intensity (MFI) of CXCR4 is observed in CD3, CD4, and/or CD8 positive F-CART as compared to CXCR4 on CD3, CD4, and/or CD8 positive C-CART. Expression of CXCR4 can be an indicator that a population of immune cells has increased migration potential to a target expressing the CXCR4 ligand, CXCL12. In an aspect, MFI of a receptor such as CXCR4, can be quantified in an engineered immune cell population to determine density of the CXCR4 receptor on a cell. Increased MFI or density of CXCR4 on an engineered immune cell, such as F-CART, can indicate increased migration potential of the cell. In some cases, migration can be measured in a population of F-CART and C-CART by determining a number of cells that migrate towards a target, for example stromal cell-derived factor 1 (SDF1), also known as C—X—C motif chemokine 12 (CXCL12). In an embodiment, a gradient of SDF-1 (human or murine) can be established in vitro or in vivo and utilized to determine migration or chemotaxis of an engineered immune cell, such as F-CART, towards a target. In an aspect, a percent of CXCR4 or MFI of CXCR4 of an F-CART can be from about 10%, 20%, 30%, 40%, 50%, 60%, 70%, 80%, 90% or up to about 100% more as compared to the percent or MFI of CXCR4 of C-CART. In an aspect, cytotoxicity is at least cytotoxicity is at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 2 fold, 2.5 fold, 3 fold, 4 fold, 5 fold, 6 fold, 7 fold, 8 fold, 9 fold or 10 fold higher in a population of cells comprising engineered immune cells as compared to a comparable population comprising engineered immune cells that undergoes an ex vivo expansion for no more than 2 weeks or 1 week when the population comprising engineered immune cells and the comparable population contact a target.

In an aspect, proliferation of a population of cells comprising engineered immune cells in vivo is enhanced and is at least 0.5 fold, 1 fold, 2 fold, 3 fold, 4 fold, 5 fold, 6 fold, 7 fold, 8 fold, 9 fold, 10 fold, 50 fold, 100 fold, 500 fold, 1000 fold, 5000 fold, or 10000 fold higher in the population comprising engineered immune cells as compared to a comparable population that undergoes an ex vivo expansion for no more than 2 weeks or 1 week when the population and comparable population contact a target. In an aspect, proliferation can be quantified in vitro using a carboxyfluorescein succinimidyl ester (CFSE) assay. In an aspect, proliferation can be quantified in vitro using a cytometer, for example using a cytometer. Variables that can be measured by cytometric methods include for example: cell size, cell count, cell morphology (shape and structure), cell cycle phase, DNA content, and the existence or absence of specific proteins on the cell surface or in the cytoplasm. A cytometer can evaluate cellular clumping that can be observed during cellular proliferation. Cellular clumping can be used as a factor to evaluate enhancement of proliferation, for example in a population of engineered cells. In another aspect, a cytometer can be used to count cells. In an aspect, a cytometer, such as a flow cytometer, can be used to quantify a number of cells in a sample, for example blood, a cell culture, bone marrow, tumor, and any combinations thereof. A flow cytometer can utilize cell surface proteins to quantify cells, such as engineered immune cells. A cellular marker that can be utilized can be: CD45, CD2, Beacon, CAR, TCR, CD3, CD4, CD8, CD62, and any combination thereof.

In an aspect, proliferation and/or persistence of engineered immune cells can be determined in vivo by quantifying a copy number of engineered immune cells in a subject using quantitative PCR (qPCR). In an aspect, a copy number of engineered immune cells is calculated as blood cell number per microliter. In an aspect, a copy number of engineered immune cells is calculated as DNA copy number per microgram. In an aspect, persistence can also be calculated in vivo.

In an aspect, bone marrow migration is at least 0.1 fold, 0.2 fold, 0.3 fold, 0.4 fold, 0.5 fold, 0.6 fold, 0.7 fold, 0.8 fold, 0.9 fold, 1 fold, 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold or 10 fold higher in the population comprising engineered immune cells as compared to a comparable population that undergoes an ex vivo expansion for no more than 2 weeks or 1 week when the population and comparable population contact a target.

In an aspect, a target can be a cancer cell or a chemokine. In some cases, a chemokine is stromal cell-derived factor-1 (SDF-1). In some cases, SDF-1 is expressed in bone marrow of a subject being administered a cellular therapy comprising engineered immune cells. In an aspect, a population comprising engineered immune cells has a greater percentage of CXCR4 positive cells as compared to a comparable population that undergoes an ex vivo expansion for no more than 2 weeks or 1 week. In an aspect, a population comprising engineered immune cells has a greater median percentage of CXCR4 positive cells that is at least 5%, 10%, 15%, 20%, 25%, 30%, 35%, 40%, 45%, 50%, 60%, 70%, 80%, 90% or 100% greater as compared to the median percentage of CXCR4 positive cells expressed by a comparable population that undergoes an ex vivo expansion for no more than 2 weeks or 1 week. In an aspect, a population comprising engineered immune cells has a greater median percentage of CXCR4 positive cells that is at least 1 fold, 2 fold, 3 fold, 4 fold, 5 fold, 6 fold, 7 fold, 8 fold, 9 fold or 10 fold greater as compared to the median percentage of CXCR4 positive cells expressed by a comparable population that undergoes an ex vivo expansion for no more than 2 weeks or 1 week. In an aspect, a population comprising engineered immune cells has a greater density of CXCR4 on a cell surface of CXCR4 positive cells as compared to the density of CXCR4 on the cell surface of a comparable population that undergoes an ex vivo expansion for no more than 2 weeks or 1 week. In an aspect, density is measured by evaluating a mean fluorescence intensity (MFI) of CXCR4 on the cell surface of the CXCR4 positive cells. CXCR4 positive cells can be CD3+, CD4+, CD8+, and any combination thereof. In some embodiments, cytotoxicity can be measured using an in vivo assay. In an aspect, a reduced cancer burden is observed in a subject when the subject is administered a population comprising engineered immune cells as compared to the cancer burden observed in a comparable subject administered a comparable population that undergoes an ex vivo expansion for no more than 2 weeks or 1 week. In an aspect, cancer burden is reduced by at least 10%, 20%, 30%, 40%, 50%, 60%, 70%, 80%, 90%, 95%, or 99% in a subject treated with a population comprising engineered immune cells as compared to a comparable subject administered a comparable population that undergoes an ex vivo expansion for no more than 2 weeks or 1 week.

In an aspect, there are a greater number of F-CART vis-a-vis C-CART in a tumor of a mammal expressing a target to which the CAR on the F-CART and C-CART shows specificity. In an aspect, there are a greater number of F-CART vs C-CART in a femur of a mammal expressing a target to which the CAR on the F-CART and C-CART shows specificity. F-CART and C-CART can be quantified in a tumor and/or a femur of a mammal via expression of CD45, CD2, and/or CAR. In an aspect, there are from about 1 fold, 2 fold, 3 fold, 4 fold, 5 fold, 6 fold, 7 fold, 8 fold, 9 fold, 10 fold, 15 fold, or up to about 20 fold more F-CART in a tumor and/or femur of a mammal as compared to C-CART.

In an aspect, cytotoxicity of an engineered immune cell, such as F-CART, is measured in an in vitro assay and compared to cytotoxicity of C-CART. In some aspects, cytotoxicity is measured in an in vivo assay. In an aspect cytotoxicity can be measured by quantifying a level of IFNγ secreted by a cell, such as a CAR-T+ cell engineered immune cell. In an aspect, an F-CART can secrete and/or express a greater level of IFNγ and/or IL-2 as compared to a C-CART or a comparable cell that undergoes an ex vivo expansion for no more than 2 weeks or 1 week when contacted with a target to which the CAR shows specificity. In an aspect, an F-CART secretes and/or expresses from about 10%, 20%, 30%, 40%, 50%, 60%, 70%, 80%, 90% or up to about 100% more IFNγ and/or IL-2 as compared to a C-CART when contacted with a cell expressing a target to which the CAR shows specificity. In an aspect, greater cytotoxicity is observed in vivo. In an aspect, cytotoxicity can be measured by quantifying a level of tumor reduction in a mammal having cancer treated with engineered immune cells, such as F-CART having a CAR receptor with specificity to the cancer. Cancer reduction in a mammal can be measured by quantifying a level of fluorescence in a mammal having tumor cells comprising a fluorescent protein. A lower fluorescence in a mammal having tumor cells comprising a fluorescent protein can indicate cytotoxicity of engineered immune cells towards the cancer. In some cases, cancer reduction in a mammal treated with F-CART can be from about 10%, 20%, 30%, 40%, 50%, 60%, 70%, 80%, 90%, or up to about 100% more as compared to a mammal treated with C-CART cells. In an aspect, a level of cellular proliferation can be quantified. Cellular proliferation can refer to cell count, clumping of cells in culture, and/or cellular division. Cellular proliferation can be quantified using an in vivo or in vitro assay. In some cases, cellular proliferation can be measured by quantifying a number of cells using a cytometer and/or via an in vitro assay such as Carboxyfluorescein succinimidyl ester (CFSE). In an aspect, an F-CART can proliferate more as compared to a C-CART when contacted with a target to which the CAR shows specificity. In an aspect, greater proliferation is observed in a population of F-CART that can be from about 10%, 20%, 30%, 40%, 50%, 60%, 70%, 80%, 90% or up to about 100% more as compared to a comparable population of C-CART when contacted with a cell expressing a target to which the CAR shows specificity. In another aspect, the present disclosure provides a method of administering a cell therapy comprising engineered immune cells expressing chimeric antigen receptor (CAR) and/or an engineered T cell receptor (TCR), comprising infusing a population of immune cells comprising the engineered immune cells into a subject in need thereof, wherein the engineered immune cells have not been subject to ex-vivo expansion for no more than 2 weeks or 1 week, or less than 13, 12, 10, 9, 8, 7 or 6 days, and wherein at least 2% of the population are stem memory T cells (TSCM). In some embodiments, at least 5% of the population are TSCM. In some embodiments, at least 10% of the population are TSCM. In some embodiments, at least 15% of the population are TSCM. In some embodiments, at least 20% of the population are TSCM. In some embodiments, at least 40% of the population are TSCM. In some embodiments, at least 50% of the population are TSCM. In some embodiments, at least 2%, 5%, 10%, 20%, 40%, 50%, or at least 60% of the population are TSCM. In some embodiments, the TSCM CD45RO⁻CD62L⁺. In some embodiments, the engineered immune cells have been subject to ex vivo expansion less than 1 week. In some embodiments, the engineered immune cells have been subject to ex vivo expansion less than 72, 48, 36, 32, or 24 hours. In some embodiments, the TCR comprises (i) a ligand binding domain specific for a ligand and (ii) a transmembrane domain. In some embodiments, the CAR comprises: (i) a ligand binding domain specific for a ligand, (ii) a transmembrane domain, and (iii) an intracellular signaling domain. In some embodiments, the ligand of the TCR or CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments, the transmembrane domain is from CD8α, CD4, CD28, CD45, PD-1 and/or CD152. In some embodiments, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least two intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the CAR further comprises a signal peptide, and wherein the signal peptide is derived from IgG1, GM-CSF and/or CD8α. In some embodiments, the engineered immune cells are from peripheral blood, cord blood, bone marrow, and/or induced pluripotent stem cells. In some embodiments, the engineered immune cells are from peripheral blood, and wherein the peripheral blood cells are T cells, NK cells, and/or NKT cells. In some embodiments, the infusing is intravenous. In some embodiments, the administering comprises infusing from about 1×10⁴/kg body weight of engineered immune cells. In some embodiments, the administering comprises infusing from about 1×10⁵/kg body weight of engineered immune cells. In some embodiments, the administering comprises infusing from about 3×10⁵/kg body weight of engineered immune cells. In some embodiments, at least 20% of the immune cells express the CAR and/or the TCR. In some embodiments, at least 40% of the immune cells express the CAR and/or the TCR. In some aspects, a method further comprises administering a secondary agent to the subject in need thereof. In some embodiments, the secondary agent is a therapeutically effective amount of an immunostimulant, immunosuppressive, anti-fungal, antibiotic, anti-angiogenic, chemotherapeutic, radioactive, and/or an antiviral. In some embodiments, the immunostimulant is IL-2. In some aspects, a method further comprises obtaining peripheral blood from the subject in need thereof after an infusion. In some embodiments, the engineered immune cells from the peripheral blood are quantified. In some embodiments, a level of a cytokine is quantified. In some embodiments, the cytokine is IL-10, IL-6, tumor necrosis factor α (TNF-α), IL-1β, IL-2, IL-4, IL-8, IL-12, and/or IFN-γ. In some embodiments, a method comprises repeating an infusion.

In some aspects, a population provided herein is further characterized in that reduced exhaustion of cells in the population is observed as compared to the exhaustion of cells in a comparable population that undergoes ex vivo expansion for no more than 2 weeks or 1 week.

In an aspect, reduced exhaustion of the population is characterized in that the population comprises less cells expressing PD1 and LAG3. In some embodiments, the population is further characterized in that a greater proliferation, cytotoxicity, and/or bone marrow migration is observed in the population as compared to the proliferation, cytotoxicity, and/or bone marrow migration of a comparable population that undergoes an ex vivo expansion for no more than 2 weeks or 1 week. In an aspect, cytotoxicity is measured in an in vitro assay. In an aspect, cytotoxicity is measured in an in vivo assay. In an aspect, cytotoxicity is at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 2 fold, 2.5 fold, 3 fold, 4 fold, 5 fold, 6 fold, 7 fold, 8 fold, 9 fold or 10 fold higher in a population of cells comprising engineered immune cells as compared to a comparable population comprising engineered immune cells that undergoes an ex vivo expansion for no more than 2 weeks or 1 week when the population comprising engineered immune cells and the comparable population contact a target.

In an aspect, proliferation in vivo and/or in vitro is at least 0.5 fold, 1 fold, 2 fold, 3 fold, 4 fold, 5 fold, 6 fold, 7 fold, 8 fold, 9 fold, 10 fold, 50 fold, 100 fold, 500 fold, 1000 fold, 5000 fold, or 10000 fold higher in the population comprising engineered immune cells as compared to a comparable population that undergoes an ex vivo expansion for no more than 2 weeks or 1 week when the population and comparable population contact a target.

In an aspect, bone marrow migration is at least 0.1 fold, 0.2 fold, 0.3 fold, 0.4 fold, 0.5 fold, 0.6 fold, 0.7 fold, 0.8 fold, 0.9 fold, 1 fold, 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold or 10 fold higher in the population comprising engineered immune cells as compared to a comparable population that undergoes an ex vivo expansion for no more than 2 weeks or 1 week when the population and comparable population contact a target.

In an aspect, a target can be a cancer cell or a chemokine. In some cases, a chemokine is stromal cell-derived factor-1 (SDF-1). In some cases, SDF-1 is expressed in bone marrow of a subject being administered a cellular therapy comprising engineered immune cells. In an aspect, a population comprising engineered immune cells has a greater percentage of CXCR4 positive cells as compared to a comparable population that undergoes an ex vivo expansion for no more than 2 weeks or 1 week. In an aspect, a population comprising engineered immune cells has a greater median percentage of CXCR4 positive cells that is at least 5%, 10%, 15%, 20%, 25%, 30%, 35%, 40%, 45%, 50%, 60%, 70%, 80%, 90% or 100% greater as compared to the median percentage of CXCR4 positive cells expressed by a comparable population that undergoes an ex vivo expansion for no more than 2 weeks or 1 week. In an aspect, a population comprising engineered immune cells has a greater median percentage of CXCR4 positive cells that is at least 1 fold, 2 fold, 3 fold, 4 fold, 5 fold, 6 fold, 7 fold, 8 fold, 9 fold or 10 fold greater as compared to the median percentage of CXCR4 positive cells expressed by a comparable population that undergoes an ex vivo expansion for no more than 2 weeks or 1 week. In an aspect, a population comprising engineered immune cells has a greater density of CXCR4 on a cell surface of CXCR4 positive cells as compared to the density of CXCR4 on the cell surface of a comparable population that undergoes an ex vivo expansion for no more than 2 weeks or 1 week. In an aspect, density is measured by evaluating a mean fluorescence intensity (MFI) of CXCR4 on the cell surface of the CXCR4 positive cells. CXCR4 positive cells can be CD3+, CD4+, CD8+, and any combination thereof. In some embodiments, cytotoxicity can be measured using an in vivo assay. In an aspect, a reduced cancer burden is observed in a subject when the subject is administered a population comprising engineered immune cells as compared to the cancer burden observed in a comparable subject administered a comparable population that undergoes an ex vivo expansion for no more than 2 weeks or 1 week. In an aspect, cancer burden is reduced by at least 10%, 20%, 30%, 40%, 50%, 60%, 70%, 80%, 90%, 95%, or 99% in a subject treated with a population comprising engineered immune cells as compared to a comparable subject administered a comparable population that undergoes an ex vivo expansion for no more than 2 weeks or 1 week. In yet another aspect, the present disclosures provides, a method of producing a population of engineered immune cells expressing a chimeric antigen receptor (CAR) and/or an engineered T cell receptor (TCR), comprising: (a) activating a population of cells comprising immune cells with an activation moiety; and concurrently (b) introducing a polynucleotide encoding for at least the CAR, wherein the CAR comprises (i) a ligand binding domain specific for a ligand, (ii) a transmembrane domain, and (iii) an intracellular signaling domain, thereby producing a population of engineered immune cells expressing the CAR. In an aspect, the activation moiety binds: a CD3/T cell receptor complex and/or provides costimulation. In an aspect, the activation moiety is any one of anti-CD3 antibody and/or anti-CD28 antibody. In some embodiments, the activation moiety is conjugated to a solid phase. In some embodiments, the solid phase is at least one of a bead, plate, and/or matrix. In an aspect, the solid phase is a bead. In an aspect, the introducing comprising transducing the population of cells with a viral vector and/or a transposon vector. In an aspect, the viral vector is a retroviral vector, a lentiviral vector and/or an adeno-associated viral vector. In some embodiments, the transposon vector is a sleeping beauty vector and/or a PiggyBac vector. In an aspect, step (a) and (b) are performed within 48 hours. In an aspect, step (a) and (b) are performed within 24 hours. In an aspect, step (a) and (b) are performed within 3 hours. In an aspect, step (a) and (b) are performed within 1 hour. In some embodiments, step (a) and (b) are performed within 30 min. In some embodiments, step (a) and (b) are performed at the same time. In some embodiments, the transducing comprises adding an infective agent. In some embodiments, an infective agent is polybrene. In some embodiments, the population of cells is seeded at a density from about 10⁴/mL to about 10⁸/mL. In some embodiments, the viral vector is plated at a mean of infectivity (MOI) from about 0.1 to about 10. In some embodiments, a method further comprises stimulating the population of cells with a cytokine. In some embodiments, the cytokine is IL2, IL7, IL15 and/or IL21. In some embodiments, the TCR comprises (i) a ligand binding domain specific for a ligand and (ii) a transmembrane domain. In some embodiments, the CAR comprises: (i) a ligand binding domain specific for a ligand, (ii) a transmembrane domain, and (iii) an intracellular signaling domain. In some embodiments, the ligand of the TCR or CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments, the transmembrane domain is from CD8α, CD4, CD28, CD45, PD-1 and/or CD152. In some embodiments, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least two intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the method further comprises enriching for the immune cells prior to the engineering. In some embodiments, the enriching comprises collecting a monocyte fraction. In some embodiments, the enriching comprises sorting the immune cells from a monocytes fraction. In some embodiments, the enriching comprises sorting the immune cells based on expression of one or more markers. In some aspects, the one or more markers comprise CD3, CD28, CD4, and/or CD8. In some aspects, the immune cells are sorted using an anti-CD3 antibody or antigen binding fragment thereof, and/or an anti-CD28 antibody or an antigen binding fragment thereof. In some embodiments, the immune cells are sorted using a bead conjugated with the anti-CD3 antibody or antigen binding fragment thereof, and/or a bead conjugated with the anti-CD28 antibody or an antigen binding fragment thereof. In some embodiments, the population of engineered immune cells is characterized in that cell memory T cells (TCM) in the population are more abundant than effector memory T cells (TEM). In an aspect, at least 2% of the population are stem memory T cells (TSCM). In an aspect, at least 5% of the population are stem memory T cells (TSCM). In an aspect, at least 10% of the population are stem memory T cells (TSCM). In an aspect, at least 15% of the population are stem memory T cells (TSCM). In some embodiments, at least 20% of the population are TSCM. In some embodiments, at least 25% of the population are TSCM. In some embodiments, at least 40% of the population are TSCM. In some embodiments, at least 50% of the population are TSCM. In some embodiments, at least 2%, 5%, 10%, 20%, 40%, 50%, or at least 60% of the population are TSCM. In an aspect, a method further comprises the step of infusing the population of engineered immune cells to a subject in need thereof within 72 hours from completion of (a) and (b). In an aspect, the population is further characterized in that reduced exhaustion of cells in the population is observed as compared to the exhaustion of cells in a comparable population that undergoes a comparable method that is absent performing (a) and (b) concurrently. In an aspect, reduced exhaustion of a population is characterized in that the population comprises fewer cells expressing PD1 and LAG3.

In an embodiment, the population is further characterized in that a greater proliferation, cytotoxicity, and/or bone marrow migration is observed in the population as compared to the proliferation, cytotoxicity, and/or bone marrow migration of a comparable population that undergoes a comparable method that is absent performing (a) and (b) concurrently. In an aspect, cytotoxicity is measured in an in vitro assay. In an aspect, cytotoxicity is measured in an in vivo assay. In an aspect, cytotoxicity is quantified and is at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 2 fold, 2.5 fold, 3 fold, 4 fold, 5 fold, 6 fold, 7 fold, 8 fold, 9 fold or 10 fold higher in a population comprising engineered immune cells as compared to a comparable population wherein (a) and (b) are performed for more than 24 hours when the population comprising engineered immune cells and comparable population contact a target.

In an aspect, proliferation in vivo and/or in vitro is at least 0.5 fold, 1 fold, 2 fold, 3 fold, 4 fold, 5 fold, 6 fold, 7 fold, 8 fold, 9 fold, 10 fold, 50 fold, 100 fold, 500 fold, 1000 fold, 5000 fold, or 10000 fold higher in the population comprising engineered immune cells as compared to a comparable population wherein (a) and (b) are performed for more than 24 hours when the population and the comparable population contact a target.

In an aspect, bone marrow migration is at least 0.1 fold, 0.2 fold, 0.3 fold, 0.4 fold, 0.5 fold, 0.6 fold, 0.7 fold, 0.8 fold, 0.9 fold, 1 fold, 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold or 10 fold higher in the population comprising engineered immune cells as compared to a comparable population wherein (a) and (b) are performed for more than 24 hours when the population and comparable population contact a target.

In an aspect, a target can be a cancer cell or a chemokine. A chemokine is stromal cell-derived factor-1 (SDF-1) that can be expressed in bone marrow of a subject receiving an administration of a population comprising engineered immune cells. In some embodiments, a population comprising engineered immune cells has a greater percentage of CXCR4 positive cells as compared to a comparable population wherein (a) and (b) are performed for more than 24 hours. In an aspect, a population comprising engineered immune cells has a greater median percentage of CXCR4 positive cells that is at least 5%, 10%, 15%, 20%, 25%, 30%, 35%, 40%, 45%, 50%, 60%, 70%, 80%, 90% or 100% greater as compared to the median percentage of CXCR4 positive cells expressed by a comparable population wherein (a) and (b) are performed for more than 24 hours. In an aspect, a population comprising engineered immune cells has a greater median percentage of CXCR4 positive cells that is at least 1 fold, 2 fold, 3 fold, 4 fold, 5 fold, 6 fold, 7 fold, 8 fold, 9 fold, or 10 fold greater as compared to the median percentage of CXCR4 positive cells expressed by a comparable population wherein (a) and (b) are performed for more than 24 hours. In an aspect, a population comprising engineered immune cells has a greater density of CXCR4 on a cell surface of the CXCR4 positive cells as compared to the density of CXCR4 on the cell surface of a comparable population wherein (a) and (b) are performed for more than 24 hours. Density of a receptor on a cell surface, such as CXCR4, can be measured by evaluating a mean fluorescence intensity (MFI) of CXCR4 on the cell surface of the CXCR4 positive cells. In an aspect, cytotoxicity is measured in an in vivo assay. In some cases, a reduced cancer burden is observed in a subject when the subject is administered a population comprising engineered immune cells as compared to the cancer burden observed in a comparable subject administered a comparable population wherein (a) and (b) are performed for more than 24 hours. In an aspect, cancer burden is reduced by at least 10%, 20%, 30%, 40%, 50%, 60%, 70%, 80%, 90%, 95%, or 99% in the subject treated with the population comprising engineered immune cells as compared to the comparable subject administered a comparable population wherein (a) and (b) are performed for more than 24 hours.

In an aspect, provided herein is a point-of-care facility comprising a cell infusion equipment configured to infuse a population of immune cells that comprises engineered immune cells that have not been subject to ex-vivo expansion for 2 or more week, or less than 13, 12, 10, 9, 8, 7, 6, 5, 4, 3, 2, 1 days, wherein the population of immune cells is further characterized in that: cell memory T cells (TCM) in the population are more abundant than effector memory T cells (TEM); or wherein at least 2% of the population are stem memory T cells (TSCM). In some embodiments, at least 5% of the population are stem memory T cells (TSCM). In some embodiments, at least 10% of the population are stem memory T cells (TSCM). In some embodiments, at least 15% of the population are stem memory T cells (TSCM). In some embodiments, at least 20% of the population are TSCM. In some embodiments, at least 40% of the population are TSCM. In some embodiments, at least 50% of the population are TSCM. In some embodiments, at least 2%, 5%, 10%, 20%, 40%, 50%, or at least 60% of the population are TSCM. In some embodiments, there are 2 fold more TCM as compared to TEM. In some embodiments, there are 4 fold more TCM as compared to TEM. In some embodiments, the engineered immune cells have been subject to ex vivo expansion less than 5 days. In some embodiments, the engineered immune cells have been subject to ex vivo expansion less than 3 days. In some embodiments, the engineered immune cells have been subject to ex vivo expansion less than 2 days. In some embodiments, the engineered immune cells have been subject to ex vivo expansion less than 1 day. In some embodiments, the immune cells are T cells, NK cells, and/or NKT cells. In some aspects, the population is further characterized in that reduced exhaustion of cells in said population is observed, and wherein said reduced exhaustion is characterized in that said population comprises less cells expressing PD1 and LAG3.

Provided herein is a point-of-care facility comprising a cell processing equipment configured to (a) receive a population of cells comprising immune cells from a subject; and (b) activate the population of immune cells with an activation moiety, and concurrently, introduce a polynucleotide encoding for at least a chimeric antigen receptor (CAR) to the immune cells, wherein the CAR comprises (i) a ligand binding domain specific for a ligand, (ii) a transmembrane domain, and (iii) an intracellular signaling domain; and (c) infuse the population of immune cells of (b) into the subject within 2 weeks or less from the time of performing (b). In some embodiments, step (c) is performed within 1 week or less from the time of performing (b). In some embodiments, the immune cells are T cells, NK cells, and/or NKT cells. In some embodiments, the ligand of the CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2, HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments, the transmembrane domain is from CD8α, CD4, CD28, CD45, PD-1 and/or CD152. In some embodiments, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least two intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the CAR further comprises a signal peptide, and wherein the signal peptide is derived from IgG1, GM-CSF and/or CD8α. In some embodiments, the immune cells are T cells, NK cells, and/or NKT cells. In some embodiments, the activation moiety binds: a CD3/T cell receptor complex and/or provides costimulation. In some embodiments, the activation moiety is any one of anti-CD3 antibody and/or anti-CD28 antibody. In some embodiments, a viral vector and/or a transposon vector comprises the polynucleotide. In an aspect, the viral vector is a retroviral vector, a lentiviral vector and/or an adeno-associated viral vector. In some embodiments, step (a) and (b) are performed within 24 hours. In some embodiments, step (a) and (b) are performed within 3 hours. In some embodiments, step (a) and (b) are performed within 1 hour. In some embodiments, step (a) and (b) are performed within 30 minutes. In some aspects, the population is further characterized in that reduced exhaustion of cells in said population is observed, and wherein the reduced exhaustion is characterized in that the population comprises fewer cells expressing PD1 and LAG3.

In an aspect, provided herein is a population of cells comprising engineered immune cells expressing a chimeric antigen receptor (CAR) and/or a T cell receptor (TCR), wherein the population is further characterized in that (i) central memory T cells (TCM) in the population are more abundant than effector memory T cells (TEM); and/or (ii) at least 2% of the population of cells are stem memory T cells (TSCM), and wherein the CAR comprises (i) a ligand binding domain specific for a ligand, (ii) a transmembrane domain, and (iii) an intracellular signaling domain. In an aspect, at least 5% of the population are TSCM. In an aspect, at least 10% of the population are TSCM. In some embodiments, there are 2 fold more TCM as compared to TEM. In some embodiments, there are 4 fold more TCM as compared to TEM. In some embodiments, the immune cells are T cells, NK cells, and/or NKT cells. In some embodiments, the ligand of the CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In an aspect, the transmembrane domain is from CD8a, CD4, CD28, CD45, PD-1 and/or CD152. In an aspect, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least two intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the CAR further comprises a signal peptide, and wherein the signal peptide is derived from IgG1, GM-CSF and/or CD8α. In an aspect, the immune cells are T cells, NK cells, and/or NKT cells. In an aspect, the population is cryopreserved. In an aspect, the population is not cryopreserved. In an aspect, the population is freshly sourced or comprises freshly sourced cells. In an aspect, the population is further characterized in that reduced exhaustion of cells in the population is observed, and wherein the reduced exhaustion is characterized in that the population comprises fewer cells expressing PD1 and LAG3.

In an aspect, provided herein is a method of treating a cancer in a subject in need thereof, comprising infusing a population of no more than about 1×10⁶ engineered immune cells expressing chimeric antigen receptor (CAR) and/or an engineered T cell receptor (TCR), wherein the engineered immune cells have not been subject to ex-vivo expansion for no more than 2 weeks or 1 week. In an aspect, the population of engineered immune cells exhibits a comparable level of anti-tumor activity in vivo as compared to a population of 10 times more engineered immune cells expressing the same chimeric antigen receptor (CAR) and/or an engineered T cell receptor (TCR) but have been subject to ex-vivo expansion for no more than 2 weeks or 1 week. In an aspect, the population of engineered immune cells have been concurrently activated and transduced with a construct expressing the CAR and/or TCR. In an aspect, the population of engineered immune cells have not been subject to ex-vivo expansion for one week. In an aspect, the population of engineered immune cells have not been subject to ex-vivo expansion for 72 hours. In an aspect, the population of no more than about 1×10⁶ engineered immune cells have been prepared from peripheral blood mononuclear cells (PMBC) via a process of concurrent activation and transduction with a construct expressing the CAR and/or TCR. In some embodiments, the infusing takes places within 1 week from concurrent activation and transduction with a construct expressing the CAR and/or TCR. In some embodiments, the concurrent activation comprises performing activation and transduction within 48 hours. In some embodiments, the concurrent activation comprises performing activation and transduction within 24 hours. In some embodiments, the concurrent activation comprises performing activation and transduction within 3 hours. In some embodiments, the concurrent activation comprises performing activation and transduction within 1 hour. In some embodiments, the concurrent activation comprises performing activation and transduction within 30 minutes. In some embodiments, the concurrent activation comprises performing activation and transduction at the same time. In some embodiments, at least 2% of the population are stem memory T cells (TSCM). In some embodiments, at least 5% of the population are stem memory T cells (TSCM). In some embodiments, at least 10% of the population are stem memory T cells (TSCM). In some embodiments, at least 15% of the population are stem memory T cells (TSCM). In some embodiments, at least 20% of the population are TSCM. In some embodiments, at least 40% of the population are TSCM. In some embodiments, at least 50% of the population are TSCM. In some embodiments, at least 2%, 5%, 10%, 20%, 40%, 50%, or at least 60% of the population are TSCM. In some embodiments, the infusing is no more than about 10⁵ engineered immune cells. In some embodiments, the infusing is no more than about 10⁴ engineered immune cells. In some embodiments, the infusing is no more than about 10 engineered immune cells. In some embodiments, the engineered immune cells are T cells, NK cells, and/or NKT cells. In some embodiments, the TCR comprises (i) a ligand binding domain specific for a ligand and (ii) a transmembrane domain. In some embodiments, the CAR comprises: (i) a ligand binding domain specific for a ligand, (ii) a transmembrane domain, and (iii) an intracellular signaling domain. In some embodiments, the ligand of the TCR or CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments, the transmembrane domain is from CD8α, CD4, CD28, CD45, PD-1 and/or CD152. In some embodiments, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some embodiments, the CAR comprises at least 2 intracellular signaling domains. In some embodiments, the CAR comprises at least 3 intracellular signaling domains. In some embodiments, the CAR further comprises a hinge. In some embodiments, the hinge is from CD28, IgG1 and/or CD8α. In some embodiments, the CAR further comprises a signal peptide, and wherein the signal peptide is derived from IgG1, GM-CSF and/or CD8α. In some embodiments, the engineered immune cells are from peripheral blood, cord blood, bone marrow, and/or induced pluripotent stem cells. In some embodiments, the engineered immune cells are from peripheral blood, and wherein the peripheral blood cells are T cells. In an aspect, a method further comprises obtaining peripheral blood from the subject in need thereof after the administering. In an aspect, the engineered immune cells in the subject are quantified from the peripheral blood. In an aspect, a level of a growth factor in the subject is quantified. In some cases, the growth factor selected from the group consisting of IL-10, IL-6, tumor necrosis factor α (TNF-α), IL-1β, IL-2, IL-4, IL-8, IL-12, and/or IFN-γ. In some embodiments a method comprises repeating an infusion. In some embodiments, the population of immune cells is allogeneic to the subject in need thereof. In some embodiments, the population of immune cells is autologous to the subject in need thereof. In some embodiments, the subject has cancer. In an aspect, cancer can be a target. In an aspect, the cancer is hematological. In an aspect, the hematological cancer is leukemia, myeloma, lymphoma, and/or a combination thereof. In some embodiments, leukemia is chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL). In some embodiments, the lymphoma is mantle cell lymphoma (MCL), T cell lymphoma, Hodgkin's lymphoma, and/or non-Hodgkin's lymphoma. In some embodiments, the cancer is a target and is solid. In some embodiments, the solid cancer target is selected from the group comprising: nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, or bladder cancer. In some embodiments, the subject was pre-treated with chemotherapy prior to the administering. In some embodiments, the chemotherapy comprises an administration of fludarabine, cyclophosphamide and/or cytarabine. In an aspect, the population is further characterized in that a greater proliferation, cytotoxicity, and/or bone marrow migration is observed in the population as compared to the proliferation, cytotoxicity, and/or bone marrow migration of a comparable population that undergoes an ex vivo expansion for no more than 2 weeks or 1 week. In an aspect, cytotoxicity is measured in an in vitro assay. In an aspect, cytotoxicity is measured in an in vivo assay.

In an aspect, provided herein is a method of administering a cell therapy comprising engineered immune cells expressing a chimeric antigen receptor (CAR) and/or an engineered T cell receptor (TCR), comprising infusing a population of immune cells comprising the engineered immune cells into a subject in need thereof, wherein the engineered immune cells have not been subject to ex vivo expansion for no more than 2 weeks or 1 week, and wherein the population is further characterized in that a greater proliferation is observed in the population as compared to the proliferation of a comparable population that undergoes ex vivo expansion for no more than 2 weeks or 1 week. In an aspect, the proliferation is at least 1 fold higher in the population as compared to the comparable population that undergoes an ex vivo expansion for no more than 2 weeks or 1 week when the population and the comparable population contact a target.

Provided herein is a method of administering a cell therapy comprising engineered immune cells expressing a chimeric antigen receptor (CAR) and/or an engineered T cell receptor (TCR), comprising infusing a population of immune cells comprising the engineered immune cells into a subject in need thereof, wherein the engineered immune cells have not been subject to ex vivo expansion for no more than 2 weeks or 1 week, and wherein the population is further characterized in that a greater cytotoxicity is observed in the population as compared to the cytotoxicity of a comparable population that undergoes ex vivo expansion for no more than 2 weeks or 1 week. In an aspect, the cytotoxicity is at least 0.1 fold, 0.2 fold, 0.3 fold, 0.4 fold, 0.5 fold, 0.6 fold, 0.7 fold, 0.8 fold, 0.9 fold, 1.0 fold, 1.1 fold, 1.2 fold, 1.3 fold, 1.4 fold, 1.5 fold, 2 fold, 2.5 fold, 3 fold, 4 fold, 5 fold, 6 fold, 7 fold, 8 fold, 9 fold or 10 fold higher in the population as compared to the comparable population that undergoes an ex vivo expansion for no more than 2 weeks or 1 week when the population and the comparable population contact a target.

Provided herein is a method of administering a cell therapy comprising engineered immune cells expressing a chimeric antigen receptor (CAR) and/or an engineered T cell receptor (TCR), comprising: infusing a population of immune cells comprising the engineered immune cells into a subject in need thereof, wherein the engineered immune cells have not been subject to ex vivo expansion for no more than 2 weeks or 1 week, and wherein the population is further characterized in that a greater bone marrow migration is observed of the population as compared to the bone marrow migration of a comparable population that undergoes ex vivo expansion for no more than 2 weeks or 1 week. In some embodiments, the bone marrow migration is at least 1 fold higher in the population as compared to the comparable population that undergoes ex vivo expansion for no more than 2 weeks or 1 week when the population and the comparable population contact a target. In an aspect, the population is further characterized in that: central memory T cells (TCM) in the population are more abundant than effector memory T cells (TEM). In an aspect, TCM are CD45RO+CD62L+. In an aspect, TEM are CD45RO+CD62L−. In an aspect, engineered immune cells have been subject to ex vivo expansion less than 5 days. In an aspect, the engineered immune cells have been subject to ex vivo expansion less than 4 days. In an aspect, the engineered immune cells have been subject to ex vivo expansion less than 72 hours. In an aspect, the engineered immune cells have been subject to ex vivo expansion less than 48 hours. In an aspect, the engineered immune cells have been subject to ex vivo expansion less than 24 hours. In an aspect, the target is a cancer cell, a ligand of the TCR or the CAR, or a chemokine. In some embodiments, the chemokine is stromal cell-derived factor-1 (SDF-1), and wherein the SDF-1 is expressed in bone marrow of the subject. In some cases, the population has a greater percentage of CXCR4 positive cells as compared to the comparable population that undergoes the ex vivo expansion for no more than 2 weeks or 1 week. In an aspect, the population has a greater median percentage of CXCR4 positive cells that is at least 10% greater as compared to the median percentage of CXCR4 positive cells expressed by the comparable population that undergoes the ex vivo expansion for no more than 2 weeks or 1 week. In an aspect, the population has a greater density of CXCR4 on a cell surface of the CXCR4 positive cells as compared to the density of CXCR4 on the cell surface of the comparable population that undergoes an ex vivo expansion for no more than 2 weeks or 1 week. In an aspect, the density is measured by evaluating a mean fluorescence intensity (MFI) of CXCR4 on the cell surface of the CXCR4 positive cells. In an aspect, the cytotoxicity is measured in an in vivo assay. In some cases, a reduced cancer burden is observed in the subject administered the population as compared to the cancer burden observed in a comparable subject administered a comparable population that undergoes an ex vivo expansion for no more than 2 weeks or 1 week. In an aspect, the cancer burden is reduced by at least 10%, 20%, 30%, 40%, 50%, 60%, 70%, 80%, 90%, 95%, or 99% in the subject treated with the population as compared to the comparable subject administered a comparable population that undergoes an ex vivo expansion for no more than 2 weeks or 1 week. In an aspect, the cancer burden is reduced by at least 0.5 fold, 1 fold, 2 fold, 3 fold, 4 fold, 5 fold, 6 fold, 7 fold, 8 fold, 9 fold, or 10 fold in the subject treated with the population as compared to the comparable subject administered a comparable population that undergoes an ex vivo expansion for no more than 2 weeks or 1 week. In an aspect, complete remission (CR) is observed in the subject administered the population as compared to the cancer burden observed in a comparable subject administered a comparable population that undergoes an ex vivo expansion for no more than 2 weeks or 1 week. In some embodiments, a partial response (PR) is observed in the subject administered the population as compared to the cancer burden observed in a comparable subject administered a comparable population that undergoes an ex vivo expansion for no more than 2 weeks or 1 week. In an aspect, the population comprises at most 1×10⁴ cells per kg/body weight of engineered immune cells. In an aspect, the population comprises from about 1×10⁴ cells per kg/body weight of engineered immune cells to at most about 1×10⁵ cells per kg/body weight of engineered immune cells. In some cases, the engineered immune cells are T cells, NK cells, and/or NKT cells. In some cases, the TCR comprises (i) a ligand binding domain specific for a ligand and (ii) a transmembrane domain. In an aspect, the CAR comprises: (i) a ligand binding domain specific for a ligand, (ii) a transmembrane domain, and (iii) an intracellular signaling domain. In an aspect, the ligand of the TCR or CAR is VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2, HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In an aspect, the transmembrane domain is from CD8α, CD4, CD28, CD45, PD-1 and/or CD152. In an aspect, the intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS). In some cases, the CAR comprises at least 2 intracellular signaling domains. In some cases, the CAR comprises at least 3 intracellular signaling domains. In some cases, the CAR further comprises a hinge. A hinge can be from CD28, IgG1 and/or CD8α. In an aspect, the engineered immune cells are from peripheral blood, cord blood, bone marrow, and/or induced pluripotent stem cells. In some cases, the engineered immune cells are from peripheral blood, and the peripheral blood cells are T cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows multiplicity of infection (MOI) vs. percent CAR-positive ratio in F-CART cells.

FIG. 2 shows a comparison between percent CAR-positive expression of conventional-CART (C-CART) and fast-CART (F-CART) generated cells.

FIG. 3 shows a phenotypic analysis using flow cytometry of control (starting material) and F-CART cells.

FIG. 4A shows a linear graphical representation of cell proliferation of anti-CD19 F-CART vs. anti-CD19 C-CART cells. FIG. 4B shows fold proliferation of anti-CD19 F-CART vs. anti-CD19 C-CART cells.

FIG. 5 depicts in vitro killing efficacy over 50 hours of anti-CD19 F-CART vs. anti-CD19 C-CART cells.

FIG. 6A shows expression of Granulocyte-macrophage colony stimulating factor (GM-CSF) in control cells (non-transduced), F-CART, and C-CART when co-cultured with Molt4 (CD19−) or Raji (CD19+) tumor cells at a ratio of 1:1. FIG. 6B shows expression of TNF-α in control cells (non-transduced), F-CART, and C-CART when co-cultured with Molt4 (CD19−) or Raji (CD19+) tumor cells at a ratio of 1:1. FIG. 6C shows expression of IL-2 in control cells, F-CART, and C-CART when co-cultured with Molt4 (CD19−) or Raji (CD19+) tumor cells at a ratio of 1:1. FIG. 6D shows expression of IFN-γ in control cells (non-transduced), F-CART, and C-CART when co-cultured with Molt4 (CD19−) or Raji (CD19+) tumor cells at a ratio of 1:1.

FIG. 7A depicts bioluminescence imaging of mice engrafted with Molt 4 or Raji tumor cells and treated with control T cells (non-transduced), C-CART, or F-CART cells at a total dose of 2e6, 5e5, or 5e4 cells/mouse. FIG. 7B shows a graphical summary of the bioluminescence imaging days after injection with 0.5×10⁶ cells/mouse of control (nontransduced) T cells, C-CART, or F-CART cells.

FIG. 8 shows change in body weight of mice engrafted with Raji tumor cells and subsequently treated with 0.5×10⁶ cells/mouse of control (non-transduced), T cells, C-CART, or F-CART cells.

FIG. 9 shows tumor volume of mice engrafted with Raji tumor cells and subsequently treated with Control, T cells, C-CART, or F-CART cells.

FIG. 10 shows a quantification of cells in the peripheral blood of mice engrafted with Raji tumor cells and subsequently treated with control (non-transduced), or F-CART cells at a high dose (2×10⁶ cells/mouse), moderate dose (5×10⁵ cells/mouse), or low dose (5×10⁴ cells/mouse).

FIG. 11A shows phenotypic analysis performed on day 6 of Lag3 vs PD-1 on F-CART and C-CART cells of three individual donors upon stimulation with K562-CD19+ cells.

FIG. 11B shows phenotypic analysis performed on day 10 of Lag3 vs PD-1 on F-CART and C-CART cells of three individual donors upon stimulation with K562-CD19+ cells. FIG. 11C shows average expression of PD1+Lag3+ cells on day 6 vs day 10 of three individual donors upon stimulation with K562-CD19+ cells.

FIG. 12 depicts flow cytometry plots showing numbers of TSCM, TCM, TEFF, and TEM cells in F-CART cells of three individual donors upon stimulation with K562-CD19+ cells.

FIG. 13A shows expansion, persistence, and copy number of FAST-CAR⁺ cells in subject XF001 up to 56 days post infusion. FIG. 13B shows body temperature of subject XF001 post infusion with FAST-CAR⁺ cells. FIG. 13C shows concentration of IL-6 in subject XF001's blood post infusion with FAST-CAR⁺ cells. FIG. 13D shows concentration of C reactive protein (CRP) in subject XF001's blood post infusion with FAST-CAR⁺ cells.

FIG. 14A shows body temperature of subject F01 post infusion with FAST-CAR⁺ cells. FIG. 14B shows FAST-CAR⁺ cell copy number, FAST-CAR⁺ copy number in the peripheral blood, and FAST-CAR⁺ copy number in the bone marrow of subject F01. FIG. 14C shows levels of growth factors (INF-γ, IL-10, sCD25, IL-6, and CRP) in the peripheral blood and body temperature of subject F01 post infusion with FAST-CAR⁺ cells.

FIG. 15 depicts treatment results and efficacy of F-CART in 9 different subjects. Cytokine release syndrome (CRS), Neurotoxicity (NT), Complete Response (CR), Mean residual disease (MRD), Allogeneic stem cell transplant (Allo-SCT).

FIG. 16A shows flow cytometry plots showing numbers of TSCM, TCM, TEFF, and TEM cells in F-CART vs C-CART cells of an individual donor. FIG. 16B shows a summary of the flow cytometry results. FIG. 16C shows a graphical summary of the percent of TSCM, TCM, TEM, and TEFF in F-CART vs C-CART of three individual donors upon stimulation with K562-CD19+ cells.

FIG. 17A shows fold expansion of F-CART vs C-CART cells on day 8, day 12, and day 18 post engineering. FIG. 17B shows percent PD-1 and LAG3 on days 6 and days 10 post engineering of F-CART and C-CART cells. FIG. 17C shows flow cytometry results of C-CART and F-CART cells of an individual donor stained with PD-1 and LAG3. FIG. 17D shows maintenance of in vitro cytotoxicity of a co-culture assay of C-CART and F-CART prepared from a healthy donor: C-CART cultured with CD19⁺ tumor cells, F-CART cultured with CD19⁺ tumor cells, non-transduced cells cultured with CD19⁺ tumor cells, and tumor only cells (Hela-CD19).

FIG. 18 shows IL-2 and IFNγ secretion of C-CART and F-CART prepared from a healthy donor: C-CART cultured with CD19⁺ tumor cells, F-CART cultured with CD19⁺ tumor cells, non-transduced cells cultured with CD19⁺ tumor cells, and media only control.

FIG. 19A shows expansion of human sample GC007F F-CART and C-CART cells. FIG. 19B shows cellular phenotype of F-CART and C-CART in sample GC007F. FIG. 19C shows a pie plot of the cellular phenotype of sample GC007F. FIG. 19D shows a graphical summary of the cellular phenotype data via percent of T cell subset. FIG. 19E shows % of PD1+LAG3+ CAR-T cells in C-CART and F-CART cells on Day 6 and Day 9, respectively.

FIG. 20A shows maintenance of in vitro cytotoxicity in a Real-Time Cell Analysis (RTCA) assay of C-CART and F-CART prepared from a patient: C-CART cultured with CD19⁺ tumor cells, F-CART cultured with CD19⁺ tumor cells, non-transduced cells cultured with CD19⁺ tumor cells, and tumor only cells (Hela-CD19). FIG. 20B shows maintenance of cytokine section in an ELISA of supernatant of the co-cultured cells. FIG. 20C shows maintenance of cytotoxicity in F-CAR vs C-CART prepared from a patient as determined in a luciferase assay.

FIG. 21A shows bioluminescence imaging of NOG mice engrafted with Raji tumor cells and treated with control (Media only), T cells, C-CART, or F-CART cells at doses of 2e6 cells/mouse (high dose) or 5e4 cells/mouse (low dose).

FIG. 22A shows tumor engraftment and treatment schematic of a leukemia mouse model. FIG. 22B shows phenotypic analysis of bone marrow of mice treated with F-CART or C-CART cells on day 10 post treatment. FIG. 22C shows number of CD45+CD2+CART+ cells in the femur of F-CART and C-CART treated mice. FIG. 22D shows expression of CXCR4 in CD4 vs. CD8 fractions of F-CART and C-CART treated mice.

FIG. 22E shows percent CXCR4 in CD4 vs. CD8 fractions of F-CART and C-CART treated mice. FIG. 22F shows MFI of the CXCR4 fraction in CD4 vs. CD8 fractions of F-CART and C-CART treated mice. FIG. 22G shows a graphical representation of results of a transwell migration assay F-CART vs. C-CART cells and mouse SDF-1α. FIG. 22H shows a graphical representation of re3sults of a transwell migration assay F-CART vs. C-CART cells and human SDF-1α.

FIG. 23A schematically illustrates presentation of a fragment of NY-ESO-1 by HLA-A*02 of a cancer cell, and recognition of the NY-ESO-1 fragment by a T cell expressing an engineered TCR. FIG. 23B illustrates a comparison of proliferative capacities of FTCRT cells and CTRCT cells, both of which are engineered to bind a fragment of NY-ESO-1. FIG. 23C illustrates a comparison of lymphocyte subpopulations in FTCRT cells and CTRCT cells, both of which are engineered to bind a fragment of NY-ESO-1. FIG. 23D illustrates a comparison of lymphocyte exhaustion in FTCRT cells and CTRCT cells, both of which are engineered to bind a fragment of NY-ESO-1. FIG. 23E illustrates a comparison of target cell cytotoxicity of FTCRT cells and CTRCT cells, both of which are engineered to bind a fragment of NY-ESO-1. FIG. 23F illustrates a different comparison of target cell cytotoxicity of FTCRT cells and CTRCT cells, both of which are engineered to bind a fragment of NY-ESO-1.

FIG. 24A illustrates CAR transduction efficiencies in GC022 cells via a conventional CART method and a FCART method. FIG. 24B illustrates cytotoxicity against target cells of CAR-expressing GC022 cells produced via a conventional CART method and a FCART method. FIG. 24C illustrates cellular expansion capacity of CAR-expressing GC022 cells produced via a conventional CART method and a FCART method. FIG. 24D illustrates cytotoxicity against target cells of CAR-expressing GC022 cells produced via a conventional CART method and a FCART method, wherein the CAR-expressing GC022 cells are expanded via antigen-stimulation. FIG. 24E illustrates a comparison of lymphocyte subpopulations in CAR-expressing GC022 cells produced via a conventional CART method and a FCART method. FIG. 24F illustrates a comparison of exhaustion in CAR-expressing GC022 cells produced via a conventional CART method and a FCART method. FIG. 24G depicts bioluminescence imaging of mice engrafted with tumor cells and treated with control T cells (non-transduced) or CAR-expressing GC022 cells produced via a conventional CART method and a FCART method. FIG. 24H shows a graphical summary of the bioluminescence imaging days after injection with control (nontransduced) T cells or CAR-expressing GC022 cells produced via a conventional CART method and a FCART method. FIG. 24I shows change in body weight of mice engrafted with tumor cells and subsequently treated with control (non-transduced) T cells or CAR-expressing GC022 cells produced via a conventional CART method and a FCART method.

### DETAILED DESCRIPTION

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell). Of particular interest are immune cells, from e.g., mammals including test animals and humans.

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen. In some cases, an antigen may be bound to a substrate (e.g., a cell membrane). Alternatively, an antigen may not be bound to a substrate (e.g., a secreted molecule, such as a secreted polypeptide).

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies). In some cases, an antibody may exhibit binding specificity to at least 1, 2, 3, 4, 5, or more different antigens. In some cases, an antibody may exhibit binding specificity to at most 5, 4, 3, 2, or 1 antigen.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif, FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The terms “target polynucleotide” and “target nucleic acid,” as used herein, refer to a nucleic acid or polynucleotide which is targeted by an actuator moiety of the present disclosure. A target polynucleotide can be DNA (e.g., endogenous or exogenous). DNA can refer to template to generate mRNA transcripts and/or the various regulatory regions which regulate transcription of mRNA from a DNA template. A target polynucleotide can be a portion of a larger polynucleotide, for example a chromosome or a region of a chromosome. A target polynucleotide can refer to an extrachromosomal sequence (e.g., an episomal sequence, a minicircle sequence, a mitochondrial sequence, a chloroplast sequence, etc.) or a region of an extrachromosomal sequence. A target polynucleotide can be RNA. RNA can be, for example, mRNA which can serve as template encoding for proteins. A target polynucleotide comprising RNA can include the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. In general, the term “target sequence” refers to a nucleic acid sequence on a single strand of a target nucleic acid. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by an actuator moiety, can result in altered gene expression and/or activity. A target polynucleotide, when targeted by an actuator moiety, can result in an edited nucleic acid sequence. A target nucleic acid can comprise a nucleic acid sequence that may not be related to any other sequence in a nucleic acid sample by a single nucleotide substitution. A target nucleic acid can comprise a nucleic acid sequence that may not be related to any other sequence in a nucleic acid sample by a 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target nucleic acid. In some embodiments, the substitution may not occur within 5, 10, 15, 20, 25, 30, 35 nucleotides of the 3′ end of a target nucleic acid.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “regulating” with reference to expression or activity, as used herein, refers to altering the level of expression or activity. Regulation can occur at the transcription level and/or translation level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “peripheral blood lymphocytes” (PBL) and its grammatical equivalents as used herein can refer to lymphocytes that circulate in the blood (e.g., peripheral blood). Peripheral blood lymphocytes can refer to lymphocytes that are not localized to organs. Peripheral blood lymphocytes can comprise T cells, NK cells, B cell, or any combinations thereof.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “TIL” or tumor infiltrating lymphocyte and its grammatical equivalents as used herein can refer to a cell isolated from a tumor. For example, a TIL can be a cell that has migrated to a tumor. A TIL can also be a cell that has infiltrated a tumor. A TIL can be any cell found within a tumor. For example, a TIL can be a T cell, B cell, monocyte, natural killer (NK) cell, or any combination thereof. A TIL can be a mixed population of cells. A population of TILs can comprise cells of different phenotypes, cells of different degrees of differentiation, cells of different lineages, or any combination thereof.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

In an aspect, the present disclosure provides a method of administering a cell therapy comprising engineered immune cells expressing a chimeric antigen receptor (CAR) and/or an engineered T cell receptor (TCR). In an aspect, the method comprises infusing a population of immune cells comprising engineered immune cells into a subject in need thereof. In an aspect, the engineered immune cells have not been subject to ex vivo expansion for 2 or more weeks. In an aspect the population is further characterized in that: central memory T cells (TCM) in the population are more abundant than effector memory T cells (TEM). In an aspect, the present disclosure provides a population of cells comprising engineered immune cells expressing a chimeric antigen receptor (CAR) and/or a T cell receptor (TCR). In an aspect, the population of cells is further characterized in that (i) central memory T cells (TCM) in the population are more abundant than effector memory T cells (TEM) and/or (ii) at least 2% of the population of cells are stem memory T cells (TSCM). In an aspect, the present disclosure provides a method of treating a cancer in a subject in need thereof, comprising infusing a population of no more than about 1×10⁶ engineered immune cells expressing a chimeric antigen receptor (CAR) and/or an engineered T cell receptor (TCR). In an aspect, a population of cells of no more than about 1×10⁶ engineered immune cells have not been subject to ex-vivo expansion for 2 or more weeks.

In some embodiments, the engineered immune cells have been subject to ex vivo expansion less than 1 week. In an aspect, the engineered immune cells have been subject to ex vivo expansion less than 6 days, less than 5 days, less than 4 days, less than 3 days, less than 2 days, less than 1 day, less than 12 hours, less than 6 hours, less than 3 hours, or are absent expansion. In an aspect, the engineered immune cells have been subject to ex vivo expansion less than 1 week, less than 72 hours, less than 48 hours, or less than 24 hours.

In an aspect, the present disclosure provides a method of treating a cancer in a subject in need thereof, comprising infusing a population of no more than about 1×10⁶ engineered immune cells expressing a chimeric antigen receptor (CAR) and/or an engineered T cell receptor (TCR). In an aspect, a population of cells of no more than about 1×10⁶ engineered immune cells have not been subject to ex-vivo expansion for 2 or more weeks. In some aspects, the population of engineered immune cells exhibit a comparable level of anti-tumor activity in vivo as compared to a population of 10 times more engineered immune cells expressing the same chimeric antigen receptor (CAR) and/or an engineered T cell receptor (TCR) but have been subject to ex-vivo expansion for 2 or more weeks. In some aspects, the population of engineered immune cells exhibit a comparable level of anti-tumor activity in vivo as compared to a population of 18 times, 15 times, 12 times, 10 times, 8 times, 6 times, 5 times, 4 times, 3 times, 2 times, 1 time more engineered immune cells expressing the same chimeric antigen receptor (CAR) and/or an engineered T cell receptor (TCR) but have been subject to ex-vivo expansion for 2 or more weeks.

In some embodiments, the engineered immune cells are phenotype and comprise central memory T cells (TCM). In some embodiments, TCM cells are CD45RO+CD62L+. In some embodiments, the engineered immune cells comprise effector memory T cells (TEM). In some embodiments, the TEM are CD45RO+CD62L−. In some embodiments, the engineered immune cells are phenotyped and comprise effector T cells (TEFF). In some embodiments, TEFF cells are CD45RO⁻CD62L⁻. In some embodiments, the engineered immune cells are phenotyped and comprise stem central memory T cells (TSCM). In some embodiments, TSCM cells are CD45RO⁻CD62L⁺. In some embodiments, a cell that can be utilized in a method provided herein can be positive or negative for a given factor. In some embodiments, a cell utilized in a method provided herein can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor Gi positive cell, a lectin-like receptor Gi negative cell, or an differentiated or de-differentiated cell thereof. The examples of factors expressed by cells is not intended to be limiting, and a person having skill in the art will appreciate that a cell may be positive or negative for any factor known in the art. In some embodiments, a cell may be positive for two or more factors. For example, a cell may be CD4+ and CD8+. In some embodiments, a cell may be negative for two or more factors. For example, a cell may be CD25−CD44−, and CD69−. In some embodiments, a cell may be positive for one or more factors, and negative for one or more factors. For example, a cell may be CD4+ and CD8−. In some aspects, a cellular marker provided herein can be utilized to select, enrich, or deplete a population of cells. In some aspects, enriching comprises selecting a monocyte fraction. In some aspects, enriching comprises sorting a population of immune cells from a monocyte fraction. In some embodiments, the cells may be selected for having or not having one or more given factors (e.g., cells may be separated based on the presence or absence of one or more factors). In some embodiments, the selected cells can also be transduced and/or expanded in vitro. The selected cells can be expanded in vitro prior to infusion. In some embodiments, selected cells can be transduced with a vector provided herein. It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells. In some cases, a cell can be a stem memory TSCM cell comprised of CD45RO (−), CCR7(+), CD45RA (+), CD62L+(L-selectin), CD27+, CD28+ and IL-7Rα+, stem memory cells can also express CD95, IL-2Rβ, CXCR3, and LFA-1, and show numerous functional attributes distinctive of stem memory cells. Cells provided herein can also be central memory TCM cells comprising L-selectin and CCR7, where the central memory cells can secrete, for example, IL-2, but not IFNγ or IL-4. Cells can also be effector memory TEM cells comprising L-selectin or CCR7 and produce, for example, effector cytokines such as IFNγ and IL-4. In some cases, a population of cells can be introduced to a subject. For example, a population of cells can be a combination of T cells and NK cells. In other cases, a population can be a combination of naïve cells and effector cells. A population of cells can be TILs.

In some embodiments, a method provided herein can include activation of a population of cells. Activation as used herein can refer to a process whereby a cell transitions from a resting state to an active state. This process can comprise a response to an antigen, migration, and/or a phenotypic or genetic change to a functionally active state. In some aspects, activation can refer to the stepwise process of T cell activation. In some cases, a T cell can require one or more signals to become activated. For example, a T cell can require at least two signals to become fully activated. The first signal can occur after engagement of a TCR by the antigen-MHC complex, and the second signal can occur by engagement of co-stimulatory molecules. Anti-CD3 antibody (or a functional variant thereof) can mimic the first signal and anti-CD28 antibody (or a functional variant thereof) can mimic the second signal in vitro.

In some aspects, a method provided herein can comprise activation of a population of cells. Activation can be performed by contacting a population of cells with a surface having attached thereto an agent that can stimulate a CD3 TCR complex associated signal and a ligand that can stimulate a co-stimulatory molecule on the surface of the cells. In particular, T cell populations can be stimulated in vitro such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. For example, a population of cells can be contacted with an anti-CD3 antibody and an anti-CD28 antibody, under conditions that can stimulate proliferation of the T cells. In some cases, 4-1BB can be used to stimulate cells. For example, cells can be stimulated with 4-1BB and IL-21 or another cytokine. For activation of either CD4 T cells or CD8 T cells, an anti-CD3 antibody and an anti-CD28 antibody can be used. For example, the agents providing a signal may be in solution or conjugated to a solid phase surface. The ratio of particles to cells may depend on particle size relative to the target cell. In further embodiments, the cells, such as T cells, can be combined with agent-coated beads, where the beads and the cells can be subsequently separated, and optionally cultured. Each bead can be coated with either anti-CD3 antibody or an anti-CD28 antibody, or in some cases, a combination of the two. In an alternative embodiment, prior to culture, the agent-coated beads and cells are not separated but are cultured together. Cell surface proteins may be conjugated by allowing paramagnetic beads to which anti-CD3 antibody and anti-CD28 antibody can be attached (3×28 beads) to contact the T cells. In one embodiment the cells and beads (for example, DYNABEADS® M-450 CD3/CD28 T paramagnetic beads at a ratio of 1:1) are combined in a buffer, for example, phosphate buffered saline (PBS) (e.g., without divalent cations such as, calcium and magnesium). Any cell concentration may be used. The mixture may be cultured for or for about several hours (e.g., about 3 hours) to or to about 14 days or any hourly integer value in between. In another embodiment, the mixture may be cultured for or for about 21 days or for up to or for up to about 21 days. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. Other additives for the growth of cells include, but are not limited to, surfactant, plasmanate, and reducing agents such as N-acetyl-cysteine and 2-mercaptoethanol. Media can include RPMI 1640, A1 M-V, DMEM, MEM, α-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, can be included only in experimental cultures, possibly not in cultures of cells that are to be infused into a subject. The target cells can be maintained under conditions necessary to support growth; for example, an appropriate temperature (e.g., 37° C.) and atmosphere (e.g., air plus 5% CO₂). In some instances, T cells that have been exposed to varied stimulation times may exhibit different characteristics. In some cases, a soluble monospecific tetrameric antibody against human CD3, CD28, CD2, or any combination thereof may be used. In some embodiments, activation can utilize an activation moiety, a costimulatory agent, and any combination thereof. In some aspects, an activation moiety binds: a CD3/T cell receptor complex and/or provides costimulation. In some aspects, an activation moiety is any one of anti-CD3 antibody and/or anti-CD28 antibody. In some aspects, a solid phase is at least one of a bead, plate, and/or matrix. In some aspects, a solid phase is a bead. Alternatively or in addition to, the activation moiety may be not be conjugated a substrate, e.g., the activation moiety may be free-floating in a medium.

In some cases, a population of cells can be activated or expanded by co-culturing with tissue or cells. A cell can be an antigen presenting cell. An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof. In some cases an artificial antigen presenting cell (aAPC) may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof. In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPCs can be combined with exogenously introduced cytokines for T cell activation, expansion, or any combination. Cells can also be expanded in vivo, for example in the subject's blood after administration of genomically transplanted cells into a subject.

In some embodiments, a method provided herein can comprise transduction of a population of cells. In some embodiments, a method comprises introducing a polynucleotide encoding for a cellular receptor such as a chimeric antigen receptor and/or a T cell receptor. In some cases, a transfection of a cell can be performed.

In some embodiments, a viral supernatant comprising a polynucleotide encoding for a cellular receptor such as a CAR and/or TCR is generated. In some embodiments, a viral vector can be a retroviral vector, a lentiviral vector and/or an adeno-associated viral vector. Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV. Additional methods for the delivery of nucleic acids to cells can be used, for example, as described in US20030087817, incorporated herein by reference.

In some embodiments, a host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. Non-limiting examples of vectors for eukaryotic host cells include but are not limited to: pBs, pQE-9 (Qiagen), phagescript, PsiXI74, pBluescript SK, pBsKS, pNH8a, pNH16a, pNH18a, pNH46a (Stratagene); pTrc99A, pKK223-3, pKK233-3, pDR54O, pRIT5 (Pharmacia). Eukaryotic: pWL-neo, pSv2cat, pOG44, pXT1, pSG (Stratagene) pSVK3, pBPv, pMSG, pSVL (Pharmiacia). Also, any other plasmids and vectors can be used as long as they are replicable and viable in a selected host. Any vector and those commercially available (and variants or derivatives thereof) can be engineered to include one or more recombination sites for use in the methods. Such vectors can be obtained from, for example, Vector Laboratories Inc., Invitrogen, Promega, Novagen, NEB, Clontech, Boehringer Mannheim, Pharmacia, EpiCenter, OnGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3 pREP4, pCEP4, and pEBVHis (Invitrogen, Corp.), and variants or derivatives thereof. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSYSPORT1 (Invitrogen) and variants or derivatives thereof. Additional vectors of interest can also include pTrxFus, pThioHis, pLEX, pTrcHis, pTrcHis2, pRSET, pBlueBa-cHis2, pcDNA3.1/His, pcDNA3.1(−)/Myc-His, pSecTag, pEBVHis, pPIC9K, pPIC3.5K, pA081S, pPICZ, pPICZA, pPICZB, pPICZC, pGAPZA, pGAPZB, pGAPZC, pBlue-Bac4.5, pBlueBacHis2, pMelBac, pSinRep5, pSinHis, pIND, pIND(SP1), pVgRXR, pcDNA2.1, pYES2, pZEr01.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; X ExCell, X gt11, pTrc99A, pKK223-3, pGEX-1X T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-lb(+), pT7Blue(R), pT7Blue-2, pCITE-4-abc(+), pOCUS-2, pTAg, pET-32L1C, pET-30LIC, pBAC-2 cp LIC, pBACgus-2 cp LIC, pT7Blue-2 LIC, pT7Blue-2, X SCREEN-1, X BlueSTAR, pET-3abcd, pET-7abc, pET9abcd, pET11 abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd (+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFPN, pEGFP-C, pEBFP, pGFPuv, pGFP, p6×His-GFP, pSEAP2-Basic, pSEAP2-Contral, pSEAP2-Promoter, pSEAP2-Enhancer, p I3 gal-Basic, p13 gal-Control, p I3 gal-Promoter, p I3 gal-Enhancer, pCMV, pTet-Off, pTet-On, pTK-Hyg, pRetro-Off, pRetro-On, pIRESlneo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, 2Xgt10, Xgt11, pWE15, and X TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phag-escript, pCAL-n-EK, pCAL-n, pCAL-c, pCAL-kc, pET-3abcd, pET-llabcd, pSPUTK, pESP-1, pCMVLacI, pOPRSVI/MCS, pOPI3 CAT, pXT1, pSG5, pPbac, pMbac, pMClneo, pMClneo Poly A, pOG44, p0G45, pFRTI3GAL, pNE0I3GAL, pRS403, pRS404, pRS405, pRS406, pRS413, pRS414, pRS415, and pRS416 from Stratagene, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp, and variants or derivatives thereof. In some embodiments, a vector can be a minicircle vector. A vector provided herein can be used to deliver a polypeptide coding for a CAR and/or TCR.

Transduction and/or transfection can be performed by any one of: non-viral transfection, biolistics, chemical transfection, electroporation, nucleofection, heat-shock transfection, lipofection, microinjection, or viral transfection. In some embodiments a provided method comprises viral transduction, and the viral transduction comprises a lentivirus. Viral particles can be used to deliver a viral vector comprising a polypeptide sequence coding for a cellular receptor into a cell ex vivo or in vivo. In some cases, a viral vector as disclosed herein may be measured as pfu (plaque forming units). In some cases, the pfu of recombinant virus or viral vector of the compositions and methods of the disclosure may be about 10⁸ to about 5×10¹⁰ pfu. In some cases, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, the viral vector of the disclosure may be measured as vector genomes. In some cases, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some cases, a viral vector provided herein can be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵ to 1×10⁷. In some cases, the MOI may be 1×10⁴ to 1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, a viral vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell.

The transfection efficiency of cells with any of the nucleic acid delivery platforms described herein, for example, transduction, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, a method can comprise adding an infective agent to a composition comprising a population of cells. An infective agent can comprise polybrene. In some aspects, an infective agent can enhance efficiency of viral infection. An infective agent can enhance viral infectivity from about 100 to 1,000 fold. Polybrene can be added to a composition at a concentration from about 5 ug to 10 ug per ml.

In some embodiments, a method provided herein can comprise a non-viral approach of introducing a cellular receptor to a cell. Non-viral approaches can include but are not limited to: CRISPR associated proteins (Cas proteins, e.g., Cas9), Zinc finger nuclease (ZFN), Transcription Activator-Like Effector Nuclease (TALEN), Argonaute nucleases, and meganucleases. Nucleases can be naturally existing nucleases, genetically modified, and/or recombinant. Non-viral approaches can also be performed using a transposon-based system (e.g. PiggyBac, Sleeping beauty).

In some embodiments, a method provided herein can utilize a PiggyBac system to introduce an exogenous polypeptide to a cell. A PiggyBac system comprises two components, a transposon and a transposase. The PiggyBac transposase facilitates the integration of the transposon specifically at ‘TTAA’ sites randomly dispersed in the genome. The predicted frequency of ‘TTAA’ in the genome is approximately 1 in every 256 base-pairs of DNA sequence. Unlike other transposons, the PB transposase also enables the excision of the transposon in a completely seamless manner, leaving no sequences or mutations behind. Furthermore, PiggyBac offers a large cargo-carrying capacity (over 200 kb has been demonstrated) with no known upper limit. PB performance levels can be increased by codon-optimization strategies, mutations, deletions, additions, substitutions, and any combination thereof. In some cases, PB can have a larger cargo (approximately 9.1-14.3 kb), a higher transposition activity, and its footprint-free characteristic can make it appealing as a gene editing tool. In some aspects, PB can comprise a few features: high efficiency transposition; large cargo; steady long-term expression; the trans-gene is integrated as a single copy; tracking the target gene in vivo by a noninvasive mark instead of traditional method such as PCR; easy to determine the integration site, and combinations thereof.

In some aspects, a method provided herein can utilize a Sleeping Beauty (SB) System to introduce a polypeptide coding for a cellular receptor to a cell. SB was engineered from ancient Tcl/mariner transposon fossils found within the Salmonid genomes by in vitro evolution. The SB ITRs (230 bp) contain imperfect direct repeats (DRs) of 32 bp in length that can serve as recognition signals for the transposase. Binding affinity and spacing between the DR elements within ITR has involved in transpositional activities. The SB transposase can be a 39 kDa protein that possess DNA binding polypeptide, a nuclear localization signal (NLS) and the catalytic domain, featured by a conserved amino acid motif (DDE). Various screens mutagenizing the primary amino acid sequence of the SB transposase resulted in hyperactive transposase versions. In some cases, a modified SB can be utilized. Modified SBs can contain mutations, deletions and additions within ITRs of the original SB transposon. Modified SBs can comprise: pT2, pT3, pT2B, pT4, SB100X, and combinations thereof. Non-limited examples of modified SBs can be selected from: SB10, SB11 (3-fold higher than SB10), SB12 (4-fold higher than SB10), HSB1-HSB5 (up to 10-fold higher than SB10), HSB13-HSB17 (HSB17 is 17-fold higher than SB10), SB100× (100-fold higher than SB10), SB150× (130-fold higher than SB10), and any combination thereof. In some cases, SB100× is 100-fold hyperactive compared to the originally resurrected transposase (SB10). In some aspects, SB transposition excision leaves a footprint (3 bp) at the cargo site. Integration occurs into TA dinucleotides of the genome, and results in target site duplications, generated by the host repair machinery. In some cases, SB appears to possess a nearly unbiased, close-to-random integration profile. Transposon integration can be artificially targeted (˜10%) to a predetermined genomic locus in wildtype systems, however in chimeric systems provided herein, SB transposon integration can be directed to a predetermined locus with efficiencies over 10%.

In some aspects, a non-viral approach may be taken to introduce an exogenous polynucleic acid to a population of cells. In some aspects, a non-viral vector or nucleic acid may be delivered without the use of a virus and may be measured according to the quantity of nucleic acid. Generally, any suitable amount of nucleic acid can be used with the compositions and methods of this disclosure. In some cases, nucleic acid may be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 pg, 10 pg, 100 pg, 200 pg, 300 pg, 400 pg, 500 pg, 600 pg, 700 pg, 800 pg, 900 pg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some cases, nucleic acid may be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 pg, 10 pg, 100 pg, 200 pg, 300 pg, 400 pg, 500 pg, 600 pg, 700 pg, 800 pg, 900 pg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some embodiments, a non-viral approach of introducing a CAR and/or TCR sequence to a cell can include electroporation. Electroporation can be performed using, for example, the Neon® Transfection System (ThermoFisher Scientific) or the AMAXA® Nucleofector (AMAXA® Biosystems). Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some embodiments, electroporation pulse voltage may be varied to optimize transfection efficiency and/or cell viability. In some cases, the electroporation voltage may be less than about 500 volts. In some cases, the electroporation voltage may be at least about 500 volts, at least about 600 volts, at least about 700 volts, at least about 800 volts, at least about 900 volts, at least about 1000 volts, at least about 1100 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 2100 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts. In some cases, the electroporation pulse voltage required for optimal transfection efficiency and/or cell viability may be specific to the cell type. For example, an electroporation voltage of 1900 volts may optimal (e.g., provide the highest viability and/or transfection efficiency) for macrophage cells. In another example, an electroporation voltage of about 1350 volts may optimal (e.g., provide the highest viability and/or transfection efficiency) for Jurkat cells or primary human cells such as T cells. In some cases, a range of electroporation voltages may be optimal for a given cell type. For example, an electroporation voltage between about 1000 volts and about 1300 volts may optimal (e.g., provide the highest viability and/or transfection efficiency) for human 578T cells. In some cases, a primary cell can be a primary lymphocyte. In some cases, a population of primary cells can be a population of lymphocytes.

In some embodiments, electroporation pulse width may be varied to optimize transfection efficiency and/or cell viability. In some cases, the electroporation pulse width may be less than about 5 milliseconds. In some cases, the electroporation width may be at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds. In some cases, the electroporation pulse width required for optimal transfection efficiency and/or cell viability may be specific to the cell type. For example, an electroporation pulse width of 30 milliseconds may optimal (e.g., provide the highest viability and/or transfection efficiency) for macrophage cells. In another example, an electroporation width of about 10 milliseconds may optimal (e.g., provide the highest viability and/or transfection efficiency) for Jurkat cells. In some cases, a range of electroporation widths may be optimal for a given cell type. For example, an electroporation width between about 20 milliseconds and about 30 milliseconds may optimal (e.g., provide the highest viability and/or transfection efficiency) for human 578T cells.

In some embodiments, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses. In some cases, the number of electroporation pulses required for optimal transfection efficiency and/or cell viability may be specific to the cell type. For example, electroporation with a single pulse may be optimal (e.g., provide the highest viability and/or transfection efficiency) for macrophage cells. In another example, electroporation with a 3 pulses may be optimal (e.g., provide the highest viability and/or transfection efficiency) for primary cells. In some cases, a range of electroporation widths may be optimal for a given cell type. For example, electroporation with between about 1 to about 3 pulses may be optimal (e.g., provide the highest viability and/or transfection efficiency) for human cells.

In some cases, the starting cell density for electroporation may be varied to optimize transfection efficiency and/or cell viability. In some cases, the starting cell density for electroporation may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells. In some cases, the starting cell density for electroporation required for optimal transfection efficiency and/or cell viability may be specific to the cell type. For example, a starting cell density for electroporation of 1.5×10⁶ cells may optimal (e.g., provide the highest viability and/or transfection efficiency) for macrophage cells. In another example, a starting cell density for electroporation of 5×10⁶ cells may optimal (e.g., provide the highest viability and/or transfection efficiency) for human cells. In some cases, a range of starting cell densities for electroporation may be optimal for a given cell type. For example, a starting cell density for electroporation between of 5.6×10⁶ and 5×10⁷ cells may optimal (e.g., provide the highest viability and/or transfection efficiency) for human cells such as T cells.

The efficiency of integration of a nucleic acid sequence encoding a CAR and/or TCR into a genome of a cell with, for example, a CRISPR, Piggy Bac, and/or Sleeping Beauty system, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

In some embodiments, a method provided herein for producing a population of engineered immune cells expressing a chimeric antigen receptor (CAR) can comprise (a) activating a population of cells comprising immune cells with an activation moiety; and concurrently (b) introducing a polynucleotide encoding for at least the CAR. In some embodiments, the CAR comprises (i) a ligand binding domain specific for a ligand, (ii) a transmembrane domain, and (iii) an intracellular signaling domain. In some embodiments step (a) and (b) are performed within 48 hours. In some embodiments, step (a) and (b) are performed within 24 hours. In some embodiments, step (a) and (b) are performed within 3 hours. In some embodiments step (a) and (b) are performed within 1 hour. In some embodiments step (a) and (b) are performed within 30 min. In some embodiments step (a) and (b) are performed at the same time. In some embodiments, step (a) and (b) can be performed within about 1 week, 6 days, 5 days, 4 days, 3 days, 2 days, 1 day, 20 hours, 15 hours, 13 hours, 10 hours, 8 hours, 6 hours, 5 hours, 4 hours, 3 hours, 2 hours, 1 hour, 45 minutes, 40 minutes, 35 minutes, 30 minutes, 25 minutes, 20 minutes, 15 minutes, 10 minutes, 5 minutes, 3 minutes, 1 minute, and/or at the same time. In some aspects, a method provided herein can further comprise infusing a population of engineered immune cells to a subject within about 1 week from completion of (a) and (b). In some aspects, a method provided herein can further comprise infusing a population of engineered immune cells to a subject within about 5 days from completion of (a) and (b). In some aspects, a method provided herein can further comprise infusing a population of engineered immune cells to a subject within about 72 hours from completion of (a) and (b). In some aspects, a method provided herein can further comprise infusing a population of engineered immune cells to a subject within about 24 hours from completion of (a) and (b). In some aspects, a method provided herein can further comprise infusing a population of engineered immune cells to a subject within about 12 hours from completion of (a) and (b). In some aspects, a method provided herein can further comprise infusing a population of engineered immune cells to a subject within about 6 hours from completion of (a) and (b). In some aspects, a method provided herein can further comprise infusing a population of engineered immune cells to a subject within about 3 hours from completion of (a) and (b).

In some embodiments, a method provided herein for producing a population of engineered immune cells expressing a chimeric antigen receptor (CAR) can comprise (a) activating a population of cells comprising immune cells with an activation moiety; and concurrently (b) introducing a polynucleotide encoding for at least the CAR. In some aspects, a method can further comprise cryopreserving the population comprising engineered immune cells expressing the CAR and/or a TCR. Cryopreservation can be performed at any time post cellular engineering. Cryopreservation can be performed from about 1 hour, 2 hours, 3 hours, 4 hours, 5 hours, 10 hours, 15 hours, 20 hours, 24 hours, 2 days, 3 days, 4 days, 5 days, 6 days, 1 week, 1.5 weeks, 2 hours, or over 2 weeks after (a) and (b). In an aspect, a population comprising engineered immune cells may be freshly sourced. For example, a freshly sourced population may have been obtained from a subject and applied the methods provided herein absent a cryopreservation.

In some embodiments, a method provided herein for producing a population of engineered immune cells expressing a chimeric antigen receptor (CAR) can comprise (a) activating a population of cells comprising immune cells with an activation moiety; and concurrently (b) introducing a polynucleotide encoding for at least the CAR, wherein (a) and (b) are performed for no more than about 48 hours. In some cases, (a) and (b) may be performed for no more than at most 48 hours, 36 hours, 24 hours, 22 hours, 20 hours, 18 hours, 16 hours, 14 hours, 12 hours, 10 hours, 9 hours, 8 hours, 7 hours, 6 hours, 5 hours, 4 hours, 3 hours, 2 hours, 1 hour, or less. In some cases, when the processes (a) and (b) do not entirely overlap with another, a total time spent in performing both (a) and (b) may be no more than 48 hours. In some cases, when the processes (a) and (b) do not entirely overlap with another, the total time spent in performing both (a) and (b) may be no more than at most 48 hours, 36 hours, 24 hours, 22 hours, 20 hours, 18 hours, 16 hours, 14 hours, 12 hours, 10 hours, 9 hours, 8 hours, 7 hours, 6 hours, 5 hours, 4 hours, 3 hours, 2 hours, 1 hour, or less.

In an example, a method provided herein for producing a population of engineered immune cells expressing a chimeric antigen receptor (CAR) can comprise (a) activating a population of cells comprising immune cells with an activation moiety; and concurrently (b) introducing a polynucleotide encoding for at least the CAR, wherein (a) and (b) are performed for no more than about 24 hours. In another example, a method provided herein for producing a population of engineered immune cells expressing a chimeric antigen receptor (CAR) can comprise (a) activating a population of cells comprising immune cells with an activation moiety; and concurrently (b) introducing a polynucleotide encoding for at least the CAR, wherein a total time spent in performing both (a) and (b) may be no more than 24 hours.

In some aspects, a method provided herein can yield more central memory T cells as compared to effector memory T cells as compared to a comparable method absent a simultaneous activation and transduction. In some aspects, there can be at least a 1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, or more increase in central memory T cells (TCM) as compared to effector memory T cells (TEM) due to generating cells utilizing a FAST-CAR method provided herein. In some aspects, there can be at most a 50 fold, 45 fold, 40 fold, 35 fold, 30 fold, 25 fold, 20 fold, 15 fold, 10 fold, 9 fold, 8 fold, 7 fold, 6 fold, 5 fold, 4 fold, 3 fold, 2 fold, 1 fold, or less increase in TCM as compared to TEM due to generating cells utilizing a FAST-CAR method provided herein. In some embodiments, a method provided herein can yield more TSCM as compared to a comparable method absent a simultaneous activation and transduction. In some embodiments, at least 2%, 3%, 4%, 5%, 6%, 7%, 8%, 9%, 10%, 15%, 20%, 25%, 30%, 35%, 40%, 45%, 50%, 60%, 70%, 80%, 90%, 95% or up to about 100% of a cellular population are TSCM. In some embodiments, at most 100%, 95%, 90%, 80%, 70%, 60%, 50%, 45%, 40%, 35%, 30%, 25%, 20%, 15%, 10%, 9%, 8%, 7%, 6%, 5%, 4%, 3%, 2%, or less of a cellular population are TSCM. A TSCM can be CD45RO⁻CD62L⁺. In some embodiments, a method provided herein can comprise administering a cell therapy comprising engineered immune cells expressing chimeric antigen receptor (CAR) and/or an engineered T cell receptor (TCR). In some aspects, a method can comprise infusing a population of immune cells comprising engineered immune cells into a subject in need thereof. In some aspects, engineered immune cells have not been subject to ex-vivo expansion for 2 or more weeks. In some aspects, engineered immune cells comprise at least 2% stem memory T cells (TSCM).

In some cases, subject cells (e.g., T cells) may not be pre-activated (e.g., by CD3/CD28 beads) prior to the simultaneous activation and transduction. In such a case, following the simultaneous activation and transduction, a duration of time for activation and transduction of the subject cells may be substantially the same.

In some embodiments, a population generating by a method provided herein can be further characterized in that it is less abundant in PD1 and LAG3. In some aspects, a population generating by a method provided herein can comprise a lower expression of cellular markers associated with exhaustion. Markers associated with cellular exhaustion comprise: PD-1, LAG3, CTLA-4, TIM-3, 2B4/CD244/SLAMF4, CD160, TIGIT, CXCR5, ICOS, to name a few. In some aspects cellular exhaustion markers can include: loss of IL-2 production, loss of proliferative capacity, loss of ex vivo cytolytic activity, Impairment in the production of TNF-alpha, IFN-gamma, and cc (beta) chemokines, Degranulation; expression of high levels of Granzyme B, Poor responsiveness to IL-7 and IL-15, Altered expression of GATA-3, Bcl-6, and Helios, In the case of CD4+, exhaustion can include a skewing towards a T Follicular Helper (Tfh) cell phenotype, secretion of IL-4, IL-6, and/or IL-21, expression of Transcription Factors: Bcl-6, IRF4, STAT4, and any combination thereof.

In some aspects, immune cells utilized in methods provided herein are T cells, NK cells, NKT cells, stem cells, induced pluripotent stem cells, B cells, to name a few. In some embodiments, cells utilized in the method provided herein are obtained from peripheral blood, cord blood, bone marrow, and/or induced pluripotent stem cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. Additionally, any T cell lines can be used. Alternatively, the cells can be obtained from a healthy donor, from a patient diagnosed with cancer, or from a patient diagnosed with an infection. In another case, the cells can be part of a mixed population of cells which present different phenotypic characteristics. A cell can also be obtained from a cell therapy bank. In an aspect, a cellular population can also be selected prior to engineering. A selection can include at least one of: magnetic separation, flow cytometric selection, antibiotic selection. In an aspect, a population of cells can comprise blood cells, such as peripheral blood mononuclear cell (PBMC), lymphocytes, monocytes or macrophages. In an aspect, immune cells can be lymphocytes, B cells, or T cells. Cells can also be obtained from whole food, apheresis, or a tumor sample of a subject. Cells can be a tumor infiltrating lymphocytes (TIL). In some cases an apheresis can be a leukapheresis. Leukapheresis can be a procedure in which blood cells are isolated from blood. During a leukapheresis, blood can be removed from a needle in an arm of a subject, circulated through a machine that divides whole blood into red cells, plasma and lymphocytes, and then the plasma and red cells are returned to the subject through a needle in the other arm. In some cases, cells are isolated after an administration of a treatment regime and cellular therapy. For example, an apheresis can be performed in sequence or concurrent with a cellular administration. In some cases, an apheresis is performed prior to and up to about 6 weeks following administration of a cellular product. In some cases, an apheresis is performed −3 weeks, −2 weeks, −1 week, 0, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or up to about 10 years after an administration of a cellular product. In some cases, cells acquired by an apheresis can undergo testing for specific lysis (for example cytotoxicity testing), cytokine release, metabolomics studies, bioenergetics studies, intracellular FACs of cytokine production, ELISA-spot assays, and lymphocyte subset analysis. In some cases, samples of cellular products or apheresis products can be cryopreserved for retrospective analysis of infused cell phenotype and function.

The methods provided herein can comprise activating a T cell and concurrently introducing (e.g., transducing or transfecting) a vector into to the T cell. The vector can be a viral vector (e.g., a lentiviral vector). The T cell can be a quiescent (e.g., resting) T cell or a non-quiescent (e.g., activated) T cell. The T cell can be an exhausted T cell. In some cases, the T cell introduced with the vector can be a population of T cells comprising quiescent T cells, non-quiescent T cell, and/or exhausted T cell. The population of T cells can be a mixture of quiescent T cells, non-quiescent T cells, and exhausted T cells.

The efficiency of transducing cells with a viral vector, while concurrently activating T cells, can be higher compared with the efficiency of transducing quiescent T cells with the viral vector without concurrent T cell activation. The efficiency of transducing cells with concurrent T cell activation can be at least 2%, 5%, 10%, 15%, 20%, 25%, 30%, 35%, 40%, 45%, 50% or more higher than the efficiency of transducing quiescent T cells without concurrent T cell activation. Since the efficiency of the concurrent transduction and activation can be high, the amount of viral vectors used in the methods provided herein may be low. The amount of viral vectors used for concurrent transduction and activation can be at least 2%, 5%, 10%, 15%, 20%, 25%, 30%, 35%, 40%, 45%, 50% or more lower than the amount used for transducing quiescent T cells without concurrent T cell activation.

The T cells used in the methods describe herein can be recovered from frozen cells (e.g., cryopreserved cells). The quiescent T cells may have a lower recovery efficiency (e.g., the percentage of recovered live cells in a population of cells) than the activated T cells. For example, the recovery efficiency of quiescent T cells 24 hours after cryopreservation may be at most about 80%, 70%, 60%, 50%, 40%, 30%, 20%, or lower. The recovery efficiency of activated T cells 24 hours after cryopreservation may be at least about 40%, 50%, 60%, 70%, 75%, 80%, 85%, 90%, 95% or higher. The recovery efficiency of the activated T cells may be maintained after 24 hours.

The engineered cells prepared using the concurrent transduction and activation methods described herein may effectively control or inhibit the tumor growth. For example, the engineered cells prepared herein may have a higher efficiency in controlling tumor growth compared with the engineered cells prepared using a method comprising transducing quiescent T cells with a viral vector without concurrent activation, under the same or substantially the same condition (e.g., animal model, dosing and experimental conditions). The engineered cells prepared using the concurrent transduction and activation methods described herein may effectively control side effects associated with administration of engineered T cells (e.g., CAR-T cells). The side effects include, but are not limited to, cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. The engineered cells prepared using the concurrent transduction and activation methods described herein can have less CRS than engineered cells prepared using a method comprising transducing quiescent T cells with a viral vector without concurrent activation. The production of a pro-inflammatory cytokine by the engineered cell prepared using the concurrent transduction and activation methods described herein can be lower compared to the engineered cells prepared using a method comprising transducing quiescent T cells with a viral vector without concurrent activation. The pro-inflammatory cytokines can be IFN-γ, TNFα, GM-CSF, IL-2 and/or IL-6.

In some embodiments, a method provided herein comprises introducing a T cell receptor (TCR) into a cell. In some embodiments, a TCR comprises (i) a ligand binding domain specific for a ligand and (ii) a transmembrane domain.

In some embodiments, a TCR can be a disulfide-linked membrane-anchored heterodimeric protein. A TCR provided herein can comprise a variable alpha (α) and/or beta (β) chain. In some aspects, the alpha and/or beta chain can be expressed as part of a complex with the invariant CD3 chain molecules. In some aspects, a TCR can comprise variable gamma (γ) and/or delta (δ) chains, referred as γδ T cells. In some aspects, a TCR chain can comprise extracellular domains: Variable (V) region, Constant (C) region, Immunoglobulin superfamily (IgSF) domain forming antiparallel β-sheets. In some embodiments, a constant region is proximal to the cell membrane, followed by a transmembrane domain and a short cytoplasmic tail, while the Variable region, such as a ligand binding domain, binds to a peptide/MHC complex. In some embodiments, a peptide can be a ligand. In some aspects, a variable domain of a TCR α-chain and β-chain can each have a hypervariable or complementarity determining regions (CDRs).

In some embodiments, a chimeric antigen receptor is provided herein. A CAR comprises: (i) a ligand binding domain specific for a ligand, (ii) a transmembrane domain, and (iii) an intracellular signaling domain. In some embodiments, a ligand binding domain of a CAR of a subject method can be linked to an intracellular signaling domain via a transmembrane domain. A transmembrane domain can be a membrane spanning segment. A transmembrane domain of a subject CAR can anchor the CAR to the plasma membrane of a cell, for example an immune cell. In some embodiments, the membrane spanning segment comprises a polypeptide. The membrane spanning polypeptide linking the ligand binding domain and the intracellular signaling domain of the CAR can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, a membrane spanning polypeptide transmits a signal from an extracellular region of a cell to an intracellular region, for via the ligand binding domain. A native transmembrane portion of CD28 can be used in a CAR. In other cases, a native transmembrane portion of CD8 alpha can also be used in a CAR. In some embodiments, a transmembrane domain of a subject CAR is from CD8α, CD4, CD28, CD45, PD-1 and/or CD152.

In some embodiments, the intracellular signaling domain of a CAR of a subject method can comprise a signaling domain, or any derivative, variant, or fragment thereof, involved in immune cell signaling. The intracellular signaling domain of a CAR can induce activity of an immune cell comprising the CAR. The intracellular signaling domain can transduce the effector function signal and direct the cell to perform a specialized function. The signaling domain can comprise signaling domains of other molecules. While usually the signaling domain of another molecule can be employed in a CAR, in many cases it is not necessary to use the entire chain. In some cases, a truncated portion of the signaling domain is used in a CAR. In some embodiments, the intracellular signaling domain comprises multiple signaling domains involved in immune cell signaling, or any derivatives, variants, or fragments thereof. For example, the intracellular signaling domain can comprise at least 2 immune cell signaling domains, e.g., at least 2, 3, 4, 5, 7, 8, 9, or 10 signaling domains. In some aspects, a subject CAR comprises at least 2 intracellular signaling domains. In some aspects, a subject CAR comprises at least 3 intracellular signaling domains.

An immune cell signaling domain can be involved in regulating primary activation of the TCR complex in either a stimulatory way or an inhibitory way. The intracellular signaling domain may be that of a T-cell receptor (TCR) complex. The intracellular signaling domain of a subject CAR can comprise a signaling domain of an Fcγ receptor (FcγR), an Fcε receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d, and Zap70. In some embodiments, the signaling domain includes an immunoreceptor tyrosine-based activation motif or ITAM. A signaling domain comprising an ITAM can comprise two repeats of the amino acid sequence YxxL/I separated by 6-8 amino acids, wherein each x is independently any amino acid, producing the conserved motif YxxL/Ix(6-8)YxxL/I. A signaling domain comprising an ITAM can be modified, for example, by phosphorylation when the ligand binding domain is bound to an epitope. A phosphorylated ITAM can function as a docking site for other proteins, for example proteins involved in various signaling pathways. In some embodiments, the primary signaling domain comprises a modified ITAM domain, e.g., a mutated, truncated, and/or optimized ITAM domain, which has altered (e.g., increased or decreased) activity compared to the native ITAM domain. In some embodiments, the intracellular signaling domain of a subject CAR comprises an FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the intracellular signaling domain comprises an FcεR signaling domain (e.g., ITAM). The FcεR signaling domain can be selected from FcεRI and FcεRII (CD23). In some embodiments, the intracellular signaling domain comprises an FcαR signaling domain (e.g., ITAM). The FcαR signaling domain can be selected from FcαRI (CD89) and Fcα/μR. In some embodiments, the intracellular signaling domain comprises a CD3ζ signaling domain. In some embodiments, the primary signaling domain comprises an ITAM of CD3ζ. In some embodiments, an intracellular signaling domain is from CD3ζ, CD28, CD54 (ICAM), CD134 (OX40), CD137 (4-1BB), GITR, CD152 (CTLA4), CD273 (PD-L2), CD274 (PD-L1), DAP10 and/or CD278 (ICOS).

In some embodiments, an intracellular signaling domain of a subject CAR comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A primary signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1(IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIR8), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the intracellular signaling domain comprises a modified ITIM domain, e.g., a mutated, truncated, and/or optimized ITIM domain, which has altered (e.g., increased or decreased) activity compared to the native ITIM domain. In some embodiments, the intracellular signaling domain comprises at least 2 ITAM domains (e.g., at least 3, 4, 5, 6, 7, 8, 9, or 10 ITAM domains). In some embodiments, the intracellular signaling domain comprises at least 2 ITIM domains (e.g., at least 3, 4, 5, 6, 7, 8, 9, or 10 ITIM domains) (e.g., at least 2 primary signaling domains). In some embodiments, the intracellular signaling domain comprises both ITAM and ITIM domains.

In some cases, the intracellular signaling domain of a subject CAR can include a co-stimulatory domain. In some embodiments, a co-stimulatory domain, for example from co-stimulatory molecule, can provide co-stimulatory signals for immune cell signaling, such as signaling from ITAM and/or ITIM domains, e.g., for the activation and/or deactivation of immune cell activity. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the costimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD5, CD53, CD58/LFA-3, CD69, CD7, CD8α, CD8β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2Rβ, IL2Rβ, IL7Rα, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the intracellular signaling domain comprises multiple costimulatory domains, for example at least two, e.g., at least 3, 4, or 5 costimulatory domains. Co-stimulatory signaling regions may provide a signal synergistic with the primary effector activation signal and can complete the requirements for activation of a T cell. In some embodiments, the addition of co-stimulatory domains to the CAR can enhance the efficacy and persistence of the immune cells provided herein.

Examples of costimulatory signaling domains are provided in Table 1.

| Gene | | | NCBI | | | Location |
|---|---|---|---|---|---|---|
| number | | | (GRCh38. | | | in |
| Symbol | Abbreviation | Name | p2) | Start | Stop | genome |
| CD27 | CD27; T14; | CD27 | 939 | 6444885 | 6451718 | 12p13 |
| | S152; Tp55; | molecule | | | | |
| | TNFRSF7; | | | | | |
| | S152. LPFS2 | | | | | |
| CD28 | Tp44; CD28; | CD28 | 940 | 203706475 | 203738912 | 2q33 |
| | CD28 antigen | molecule | | | | |
| TNFRSF9 | ILA; 4-1BB; | tumor | 3604 | 7915871 | 7943165 | 1p36 |
| | CD137; | necrosis | | | | |
| | CDw137 | factor | | | | |
| | | receptor | | | | |
| | | superfamily, | | | | |
| | | member 9 | | | | |
| TNFRSF4 | OX40; | tumor | 7293 | 1211326 | 1214638 | 1p36 |
| | ACT35; | necrosis | | | | |
| | CD134; | factor | | | | |
| | IMD16; | receptor | | | | |
| | TXGP1L | superfamily, | | | | |
| | | member 4 | | | | |
| TNFRSF8 | CD30; Ki-1; | tumor | 943 | 12063330 | 12144207 | 1p36 |
| | D1S166E | necrosis | | | | |
| | | factor | | | | |
| | | receptor | | | | |
| | | superfamily, | | | | |
| | | member 8 | | | | |
| CD40LG | IGM; IMD3; | CD40 | 959 | 136648177 | 136660390 | Xq26 |
| | TRAP; gp39; | ligand | | | | |
| | CD154; | | | | | |
| | CD40L; | | | | | |
| | HIGM1; T- | | | | | |
| | BAM; | | | | | |
| | TNFSF5; | | | | | |
| | hCD40L | | | | | |
| ICOS | AILIM; | inducible T- | 29851 | 203936731 | 203961579 | 2q33 |
| | CD278; | cell co- | | | | |
| | CVID1 | stimulator | | | | |
| ITGB2 | LAD; CD18; | integrin, | 3689 | 44885949 | 44928873 | 21q22.3 |
| | MF17; MFI7; | beta 2 | | | | |
| | LCAMB; | (complement | | | | |
| | LFA-1; | component 3 | | | | |
| | MAC-1 | receptor 3 | | | | |
| | | and 4 | | | | |
| | | subunit) | | | | |
| CD2 | T11; SRBC; | CD2 | 914 | 116754435 | 116769229 | 1p13.1 |
| | LFA-2 | molecule | | | | |
| CD7 | GP40; TP41; | CD7 | 924 | 82314865 | 82317604 | 17q25.2- |
| | Tp40; LEU-9 | molecule | | | | q25.3 |
| KLRC2 | NKG2C; | killer cell | 3822 | 10430599 | 10435993 | 12p13 |
| | CD159c; | lectin-like | | | | |
| | NKG2-C | receptor | | | | |
| | | subfamily C, | | | | |
| | | member 2 | | | | |
| TNFRSF18 | AITR; GITR; | tumor | 8784 | 1203508 | 1206709 | 1p36.3 |
| | CD357; | necrosis | | | | |
| | GITR-D | factor | | | | |
| | | receptor | | | | |
| | | super family, | | | | |
| | | member 18 | | | | |
| TNFRSF14 | TR2; ATAR; | tumor | 8764 | 2556365 | 2565622 | 1p36.32 |
| | HVEA; | necrosis | | | | |
| | HVEM; | factor | | | | |
| | CD270; | receptor | | | | |
| | LIGHTR | superfamily, | | | | |
| | | member 14 | | | | |
| HAVCR1 | TIM; KIM1; | hepatitis A | 26762 | 156979480 | 157069527 | 5q33.2 |
| | TIM1; CD365; | virus cellular | | | | |
| | HAVCR; | receptor 1 | | | | |
| | KIM-1; TIM- | | | | | |
| | 1; TIMD1; | | | | | |
| | TIMD-1; | | | | | |
| | HAVCR-1 | | | | | |
| LGALS9 | HUAT; | lectin, | 3965 | 27631148 | 27649560 | 17q11.2 |
| | LGALS9A, | galactoside- | | | | |
| | Galectin-9 | binding, | | | | |
| | | soluble, 9 | | | | |
| CD83 | BL11; HB15 | CD83 | 9308 | 14117256 | 14136918 | 6p23 |
| | | molecule | | | | |

As an example, a CAR can comprise a CD3 zeta-chain (sometimes referred to as a 1st generation CAR). As another example, a CAR can comprise a CD-3 zeta-chain and a single co-stimulatory domain (for example, CD28 or 4-1BB) (sometimes referred to as a 2nd generation CAR). As another example, a CAR can comprise a CD-3 zeta-chain and two co-stimulatory domains (CD28/OX40 or CD28/4-1BB) (sometimes referred to as a 3rd generation CAR). Together with co-receptors such as CD8, these signaling moieties can produce downstream activation of kinase pathways, which support gene transcription and functional cellular responses.

In some embodiments, a subject CAR can comprise a hinge or a spacer. The hinge or the spacer can refer to a segment between the ligand binding domain and the transmembrane domain. In some embodiments, a hinge can be used to provide flexibility to a ligand binding domain, e.g., scFv. In some embodiments, a hinge can be used to detect the expression of a CAR on the surface of a cell, for example when antibodies to detect the scFv are not functional or available. In some cases, the hinge is derived from an immunoglobulin molecule and may require optimization depending on the location of the first epitope or second epitope on the target. In some cases, a hinge may not belong to an immunoglobulin molecule but instead to another molecule such the native hinge of a CD8 alpha molecule. A CD8 alpha hinge can contain cysteine and proline residues which many play a role in the interaction of a CD8 co-receptor and MHC molecule. In some embodiments, a cysteine and proline residue can influence the performance of a CAR and may therefore be engineered to influence a CAR performance.

A hinge can be of any suitable length. In some embodiments, a CAR's hinge can be size tunable and can compensate to some extent in normalizing the orthogonal synapse distance between a CAR expressing cell and a target cell. This topography of the immunological synapse between the CAR expressing cell and target cell can also define a distance that cannot be functionally bridged by a CAR due to a membrane-distal epitope on a cell-surface target molecule that, even with a short hinge CAR, cannot bring the synapse distance in to an approximation for signaling. Likewise, membrane-proximal CAR target antigen epitopes have been described for which signaling outputs are only observed in the context of a long hinge CAR. A hinge disclosed herein can be tuned according to the single chain variable fragment region that can be used. In some aspects, a hinge can be from CD28, IgG1 and/or CD8α.

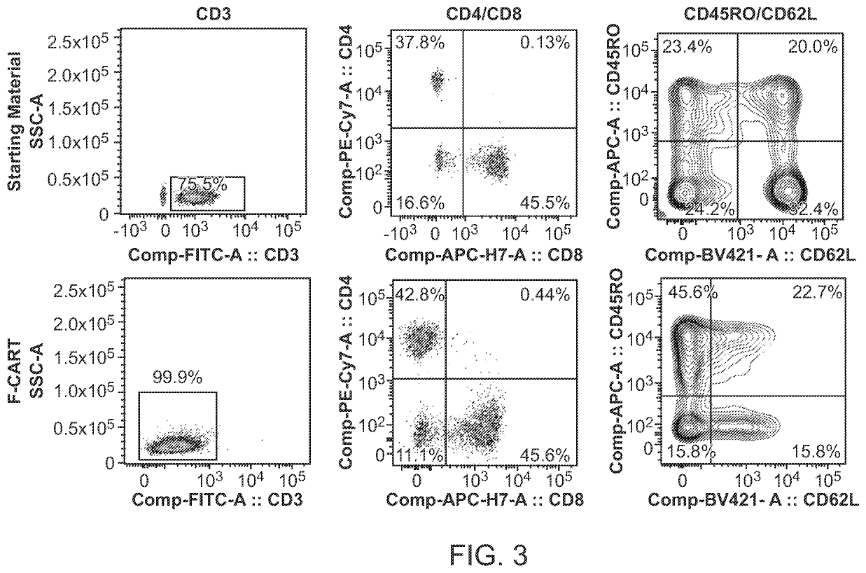

As an example, a CAR can comprise an extracellular ligand binding domain, a transmembrane domain, and an intracellular signaling domain, is illustrated in FIG. 3. A CAR may generally comprise a ligand binding domain derived from single chain antibody, hinge domain (H) or spacer, transmembrane domain (TM) providing anchorage to plasma membrane, and signaling domains responsible of T-cell activation. A CAR can comprise an immune cell signaling domain, such as a CD3ζ-chain. A CAR can comprise an immune cell signaling domains and a first costimulatory domain, such as CD3ζ-chain and 4-1BB. A CAR can comprise an immune cell signaling domain and at least two costimulatory domains, such as CD3ζ-chain, 4-1BB, and OX40. In some embodiments, a universal CAR can also be utilized in a method provided herein. A universal CAR can comprise an intracellular signaling domain fused to a protein domain that binds a tag (e.g., fluorescein isothiocyanate or biotin) on a monoclonal antibody. Various combinations of immune cell signaling domains and costimulatory domains may be utilized in a subject CAR. In some embodiments, immune cell signaling domains may be from CD3, CD4, and/or CD8. Costimulatory domains can be from 4-1BB, OX40, CD28, and the like.

In some embodiments, a ligand of a subject TCR or a subject CAR can be or can be a portion of any one of: VEGFR-2, CD19, CD20, CD30, CD22, CD25, CD28, CD30, CD33, CD52, CD56, CD80, CD86, CD81, CD123, cd171, CD276, B7H4, BCMA, CD133, EGFR, GPC3, PMSA, CD3, CEACAM6, c-Met, EGFRvIII, ErbB2, ErbB3, HER3, ErbB4/HER-4, EphA2, IGF1R, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2′ HVEM, MAGE-A, mesothelin, NY-ESO-1, RANK, ROR1, TNFRSF4, CD40, CD137, TWEAK-R, LTPR, LIFRP, LRP5, MUC1, TCRα, TCRβ, TLR7, TLR9, PTCH1, WT-1, Robol, Frizzled, OX40, CD79, Notch-1-4, and/or Claudin18.2. In some embodiments, a ligand of a subject TCR or a subject CAR can be or can be a portion of any one of a cancer cell, an endogenous cell, a cell of a vasculature, a cell of a tumor microenvironment, and any combination thereof.

In some embodiments, a subject CAR further comprises a signal peptide. In some aspects, the CAR of the present disclosure may comprise a signal peptide so that when the CAR is expressed inside a cell, such as an immune cell, the nascent protein is directed to the endoplasmic reticulum and subsequently to the cell surface, where it can be expressed. The core of the signal peptide may contain a long stretch of hydrophobic amino acids that has a tendency to form a single alpha-helix. The signal peptide may begin with a short positively charged stretch of amino acids, which can assist to enforce proper topology of the polypeptide during translocation. At the end of the signal peptide there can be a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule. In some embodiments, a subject CAR may have the general formula: Signal peptide-ligand binding domain-spacer domain-transmembrane domain/intracellular T cell signaling domain. A signal peptide can be or can be derived from IgG1, GM-CSF and/or CD8α.

In some embodiments, a method can comprise an administration comprising an infusion of an engineered cell provided herein. In some aspects, an infusion can be intravenous. In some embodiments, the administering comprises infusing from about 1×10²/kg body weight of engineered immune cells. In some embodiments, the administering comprises infusing from about 1×10³/kg body weight. In some embodiments, the administering comprises infusing from about 1×10⁴/kg body weight. In some aspects, an administering comprises infusing from about 1×10⁵/kg body weight. In some aspects, an administering comprises infusing from about 3×10⁵/kg body weight. In some aspects, an administering comprises infusing from about 1×10⁵/kg body weight to about 3×10⁵/kg body weight. In some aspects, an administering comprises infusing from about 0.5×10⁵/kg body weight to about 1×10⁵/kg body weight. In some aspects, an administering comprises infusing from about 1×10⁴/kg body weight to about 4×10⁵/kg body weight. In some aspects, an administering comprises infusing from about 0.5×10⁵/kg body weight to about 1×10⁵/kg body weight. In some aspects, an administering comprises infusing from about 0.5×10⁵/kg body weight to about 1.5×10⁵/kg body weight. In some embodiments, the administering comprises infusing from about 1×10³/kg body weight.

In some embodiments, a total of about 5×10¹⁰ cells are administered to a subject. In some cases, about 5×10¹⁰ cells represent the median amount of cells administered to a subject. In some embodiments, about 5×10¹⁰ cells are necessary to affect a therapeutic response in a subject. In some embodiments, a subject can be administered a total concentration or a dose (cells/kg body weight) with at least about 1×10⁶ cells, at least about 2×10⁶ cells, at least about 3×10⁶ cells, at least about 4×10⁶ cells, at least about 5×10⁶ cells, at least about 6×10⁶ cells, at least about 6×10⁶ cells, at least about 8×10⁶ cells, at least about 9×10⁶ cells, 1×10⁷ cells, at least about 2×10⁷ cells, at least about 3×10⁷ cells, at least about 4×10⁷ cells, at least about 5×10⁷ cells, at least about 6×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 6×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 6×10¹¹ cells, at least about 8×10¹¹ cells, at least about 9×10¹¹ cells, or at least about 1×10¹² cells are administered to a subject or dosed according to body weight (cells/kg body weight). For example, about 5×10¹⁰ cells may be administered to a subject. In another example, starting with 3×10⁶ cells, the cells may be expanded to about 5×10¹⁰ cells and administered to a subject. In some cases, cells are expanded to sufficient numbers for therapy. For example, 5×10⁷ cells can undergo rapid expansion to generate sufficient numbers for therapeutic use. In some embodiments, a total of less than about 1×10⁶ cells are administered to a subject. In some cases, about 1×10⁶ cells represent the median amount of cells administered to a subject. In some cases, at most about 9×10⁵ cells, at most about 8×10⁵ cells, at most about 7×10⁵ cells, at most about 6×10⁵ cells, at most about 5×10⁵ cells, at most about 4×10⁵ cells, at most about 3×10⁵ cells, at most about 2×10⁵ cells, at most about 1×10⁵ cells, at most about 9×10⁴ cells, at most about 8×10⁴ cells, at most about 7×10⁴ cells, at most about 6×10⁴ cells, at most about 5×10⁴ cells, at most about 4×10⁴ cells, at most about 3×10⁴ cells, at most about 2×10⁴ cells, at most about 1×10⁴ cells, at most about 9×10³ cells, at most about 8×10³ cells, at most about 7×10³ cells, at most about 6×10³ cells, at most about 5×10³ cells, at most about 4×10³ cells, at most about 3×10³ cells, at most about 2×10³ cells, or at most about 1×10³ cells are administered to a subject or dosed according to body weight (cells/kg body weight).

In some embodiments, a method provided herein is absent a cellular expansion. In some aspects, engineered cells, such as immune cells, have been subject to ex vivo expansion less than 3 weeks. In some aspects, engineered cells, such as immune cells, have been subject to ex vivo expansion less than 2 weeks. In some aspects, engineered cells, such as immune cells, have been subject to ex vivo expansion less than 1 week. In some aspects, engineered cells, such as immune cells, have been subject to ex vivo expansion less than 5 days. In some aspects, engineered cells, such as immune cells, have been subject to ex vivo expansion less than 3 days. In some aspects, engineered cells, such as immune cells, have been subject to ex vivo expansion less than 2 days. In some aspects, engineered cells, such as immune cells, have been subject to ex vivo expansion less than 1 day. In some cases, the total number of cells (e.g., F-CART cells) may be administered to the subject via a single administration. Alternatively, the total number of cells (e.g., F-CART cells) may be administered to the subject in a plurality of rounds, such as, for example, via two separate administrations separated by at least 1 hours, 2 hours, 3 hours, 4 hours, 5 hours, 6 hours, 7 hours, 8 hours, 9 hours, 10 hours, 11 hours, 12 hours, 16 hours, 20 hours, 24 hours, or more.

In some cases, sufficient numbers for therapeutic use can be about 5×10⁴. Any number of cells can be infused for therapeutic use and those cells can be comprised in a pharmaceutical composition. For example, a patient may be infused with a number of cells between 1×10⁴ to 5×10¹²per kg/body weight inclusive. A patient may be infused with as many cells that can be generated for them. In some aspects, generation of cells is absent an expansion. In some cases, cells that are infused into a patient are not all engineered. For example, at least 90% of cells that are infused into a patient can be engineered. In other instances, at least 40% of cells that are infused into a patient can be engineered. The amount of cells that are necessary to be therapeutically effective in a patient may vary depending on the viability of the cells, and the efficiency with which the cells have been modified. In some cases, the product (e.g., multiplication) of the viability of cells post genetic modification may correspond to the therapeutic aliquot of cells available for administration to a subject. In some cases, an increase in the viability of cells post modification may correspond to a decrease in the amount of cells that are necessary for administration to be therapeutically effective in a patient. In some aspects, at least about 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or up to about 100% of said immune cells express said CAR and/or said TCR. In some aspects, engineered cells can be selected for administration. In some aspects, at least 20% of immune cells express a CAR and/or a TCR. In some aspects, at least 25% of immune cells express a CAR and/or a TCR. In some aspects, at least 30% of immune cells express a CAR and/or a TCR. In some aspects, at least 40% of immune cells express a CAR and/or a TCR.

In some embodiments, a subject method can further comprise administering a secondary agent to a subject in need thereof. A secondary agent can be a therapeutically effective amount of an immunostimulant, immunosuppressive, anti-fungal, antibiotic, anti-angiogenic, chemotherapeutic, radioactive, and/or an antiviral. Secondary agents can be pharmaceutical compositions.

In some embodiments, an immunostimulant can be introduced to cells or to a subject. An immunostimulant can be specific or non-specific. A specific immunostimulant can provide antigenic specificity such as a vaccine or an antigen. A non-specific immunostimulant can augment an immune response or stimulate an immune response. A non-specific immunostimulant can be an adjuvant. Immunostimulants can be any one of vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with cells of the provided methods. Cytokines can be utilized to boost cytotoxic T lymphocytes (including adoptively transferred tumor-specific cytotoxic T lymphocytes) to expand within a tumor microenvironment. In some cases, IL-2 can be used to facilitate expansion of the cells described herein. Cytokines such as IL-15 can also be employed. Other relevant cytokines in the field of immunotherapy can also be utilized, such as IL-2, IL-7, IL-12, IL-15, IL-21, or any combination thereof. In some cases, IL-2, IL-7, and IL-15 are used to culture cells of the invention. An interleukin can be IL-2, or aldeskeukin.

In some aspects, an immunostimulant can be administered to subject. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg. An immunostimulant (e.g., aldesleukin) can be administered from 1 dose to about 14 doses. An immunostimulant (e.g., aldesleukin) can be administered from at least about 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, or up to about 20 doses. In some cases, an immunostimulant such as aldesleukin can be administered from about 1 dose to 3 doses, from 3 doses to 5 doses, from 5 doses, to 8 doses, from 8 doses to 10 doses, from 10 doses to 14 doses, from 14 doses to 20 doses. In some cases, aldeskeukin is administered over 20 doses. In some cases, an immunostimulant, such as aldesleukin, can be administered in sequence or concurrent with a cellular administration. For example, an immunostimulant can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14. In some cases, an immunostimulant, such as aldesleukin, is administered from day 0 to day 4 after administration of a population of cells. In some cases, an immunostimulant (e.g., aldesleukin) is administered over a period of about 10 min, 15 min, 20 min, 30 min, 40 min, 50 min, 1 hour, 2 hours or up to about 3 hours. In some cases, an immunostimulant (e.g., aldesleukin) can be administered from about 24 hours prior to an administration of engineered cell to about 4 days after an administration of engineered cells. An immunostimulant (e.g., aldesleukin) can be administered from day −7, −6, −5, −4, −3, −2, −1, 0, 1, 2, 3, 4, 5, 6, 7, 8, 9, 10, 11, 12, 13, 14, 15, 16, 17, 18, 19, or up to about 20 days after an administration of engineered cells.

In some embodiments, an immunostimulant is a colony stimulating factor. A colony stimulating factor can be G-CSF (filgrastim). Filgrastim can be stored in 300 mcg/ml and 480 ug/1.6 ml vials. Filgrastim can be administered daily as a subcutaneous injection. A filgrastim administration can be from about 5 mcg/kg/day. A filgrastim administration can be from about 1 mcg/kg/day, a filgrastim administration can be from about 2 mcg/kg/day, a filgrastim administration can be from about 3 mcg/kg/day, a filgrastim administration can be from about 4 mcg/kg/day, a filgrastim administration can be from about 5 mcg/kg/day, a filgrastim administration can be from about 6 mcg/kg/day, a filgrastim administration can be from about 7 mcg/kg/day, a filgrastim administration can be from about 8 mcg/kg/day, a filgrastim administration can be from about 9 mcg/kg/day, a filgrastim administration can be from about 10 mcg/kg/day. In some cases, Filgrastim can be administered at a dose ranging from about 0.5 mcg/kg/day to about 1.0 mcg/kg/day, from about 1.0 mcg/kg/day to 1.5 mcg/kg/day, from about 1.5 mcg/kg/day to about 2.0 mcg/kg/day, from about 2.0 mcg/kg/day to about 3.0 meg/kg/day, from about 2.5 meg/kg/day to about 3.5 meg/kg/day, from about 3.5 meg/kg/day to about 4.0 mcg/kg/day, from about 4.0 mcg/kg/day to about 4.5 meg/kg/day. Filgrastim administration can continue daily until neutrophil count is at least about 1.0×10⁹/L×3 days or at least about 5.0×10⁹/L. An immunostimulant such as Filgrastim can be administered from day −7, −6, −5, −4, −3, −2, −1, 0, 1, 2, 3, 4, 5, 6, 7, 8, 9, 10, 11, 12, 13, 14, 15, 16, 17, 18, 19, or up to about 20 days after an administration of engineered cells.

In some embodiments, a method can further comprise administering an immunosuppressive agent to a subject. In some cases, a subject may receive an immunosuppressive agent as part of a therapy regime. An immunosuppressive agent can refer to a radiotherapeutic, a biologic, or a chemical agent. In some cases, an immunosuppressive agent can include a chemical agent. For example, a chemical agent can comprise at least one member from the group consisting of: cyclophosphamide, mechlorethamine, chlorambucil, melphalan, ifosfamide, thiotepa, hexamethylmelamine, busulfan, fludarabine, nitrosoureas, platinum, methotrexate, azathioprine, mercaptopurine, procarbazine, dacarbazine, temozolomide, carmustine, lomustine, streptozocin, fluorouracil, dactinomycin, anthracycline, mitomycin C, bleomycin, and mithramycin. A chemical agent can be cyclophosphamide or fludarabine.

Additionally, immunosuppressive agents can include glucocorticoids, cytostatic, antibodies, anti-immunophilins, or any derivatives thereof. A glucocorticoid can suppress an allergic response, inflammation, and autoimmune conditions. Glucocorticoids can be prednisone, dexamethasone, and hydrocortisone. Immunosuppressive therapy can comprise any treatment that suppresses the immune system. Immunosuppressive therapy can help to alleviate, minimize, or eliminate transplant rejection in a recipient. For example, immunosuppressive therapy can comprise immuno-suppressive drugs. Immunosuppressive drugs that can be used before, during and/or after transplant, but are not limited to, MMF (mycophenolate mofetil (Cellcept)), ATG (anti-thymocyte globulin), anti-CD154 (CD40L), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. Furthermore, one or more than one immunosuppressive agents/drugs can be used together or sequentially. One or more than one immunosuppressive agents/drugs can be used for induction therapy or for maintenance therapy. The same or different drugs can be used during induction and maintenance stages. In some cases, daclizumab (Zenapax) can be used for induction therapy and tacrolimus (Prograf) and sirolimus (Rapimune) can be used for maintenance therapy. Daclizumab (Zenapax) can also be used for induction therapy and low dose tacrolimus (Prograf) and low dose sirolimus (Rapimune) can be used for maintenance therapy. Immunosuppression can also be achieved using non-drug regimens including, but not limited to, whole body irradiation, thymic irradiation, and full and/or partial splenectomy.

In some cases, a cytostatic agent can be administered for immunosuppression. Cytostatic agents can inhibit cell division. A cytostatic agent can be a purine analog. A cytostatic agent can be an alkylating agent, an antimetabolite such as methotrexate, azathioprine, or mercaptopurine. A cytostatic agent can be at least one of cyclophosphamide, mechlorethamine, chlorambucil, melphalan, ifosfamide, thiotepa, hexamethylmelamine, busulfan, fludarabine, nitrosoureas, platinum, methotrexate, azathioprine, mercaptopurine, procarbazine, dacarbazine, temozolomide, carmustine, lomustine, streptozocin, fluorouracil, dactinomycin, anthracycline, mitomycin C, bleomycin, and mithramycin.

In some cases, an immunosuppressive agent such as fludarabine can be administered as part of a treatment regime. Fludarabine phosphate can be a synthetic purine nucleoside that differs from physiologic nucleosides in that the sugar moiety can be arabinose instead of ribose or deoxyribose. Fludarabine can be a purine antagonist antimetabolite. Fludarabine can be supplied in a 50 mg vial as a fludarabine phosphate powder in the form of a white, lyophilized solid cake. Following reconstitution with 2 mL of sterile water for injection to a concentration of 25 mg/ml, the solution can have a pH of 7.7. The fludarabine powder can be stable for at least 18 months at 2-8° C.; when reconstituted, fludarabine is stable for at least 16 days at room temperature. Because no preservative is present, reconstituted fludarabine will typically be administered within 8 hours. Specialized references should be consulted for specific compatibility information. Fludarabine can be dephosphorylated in serum, transported intracellularly and converted to the nucleotide fludarabine triphosphate; this 2-fluoro-ara-ATP molecule is thought to be required for the drug's cytotoxic effects. Fludarabine inhibits DNA polymerase, ribonucleotide reductase, DNA primase, and may interfere with chain elongation, and RNA and protein synthesis. Fludarabine can be administered as an IV infusion in 100 ml 0.9% sodium chloride, USP over 15 to 30 minutes. The doses will be based on body surface area (BSA). If patient is obese (BMI>35) drug dosage will be calculated using practical weight. In some cases, an immunosuppressive agent such as fludarabine can be administered from about 20 mg/m² to about 30 mg/m² of body surface area of a subject. In some cases, an immunosuppressive agent such as fludarabine can be administered from about 5 mg/m² to about 10 mg/m² of body surface area of a subject, from about 10 mg/m² to about 15 mg/m² of body surface area of a subject, from about 15 mg/m² to about 20 mg/m² of body surface area of a subject, from about 20 mg/m² to about 25 mg/m² of body surface area of a subject, from about 25 mg/m² to about 30 mg/m² of body surface area of a subject, from about 30 mg/m² to about 40 mg/m² of body surface area of a subject. In some cases, an immunosuppressive agent such as fludarabine can be administered from about 1 mg/m², 2 mg/m², 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 31 mg/m², 32 mg/m², 33 mg/m², 34 mg/m², 35 mg/m², 36 mg/m², 37 mg/m², 38 mg/m², 39 mg/m², 40 mg/m², 41 mg/m², 42 mg/m², 43 mg/m², 44 mg/m², 45 mg/m², 46 mg/m², 47 mg/m², 48 mg/m², 49 mg/m², 50 mg/m², 51 mg/m², 52 mg/m², 53 mg/m², 54 mg/m², 55 mg/m², 56 mg/m², 57 mg/m², 58 mg/m², 59 mg/m², 60 mg/m², 61 mg/m², 62 mg/m², 63 mg/m², 64 mg/m², 65 mg/m², 66 mg/m², 67 mg/m², 68 mg/m², 69 mg/m², 70 mg/m², 71 mg/m², 72 mg/m², 73 mg/m², 74 mg/m², 75 mg/m², 76 mg/m², 77 mg/m², 78 mg/m², 79 mg/m², 80 mg/m², 81 mg/m², 82 mg/m², 83 mg/m², 84 mg/m², 85 mg/m², 86 mg/m², 87 mg/m², 88 mg/m², 89 mg/m², 90 mg/m², 91 mg/m², 92 mg/m², 93 mg/m², 94 mg/m², 95 mg/m², 96 mg/m², 97 mg/m², 98 mg/m², 99 mg/m², up to about 100 mg/m² of body surface area of a subject. In some cases, an immunosuppressive agent such as fludarabine is at a dose of 25 mg/m² in 100 ml 0.9% sodium chloride, USP and infused over about 15 to about 30 minutes.

In some cases, an immunosuppressive agent such as cyclophosphamide can be administered as part of a treatment regime. Cyclophosphamide can be a nitrogen mustard-derivative alkylating agent. Following conversion to active metabolites in the liver, cyclophosphamide functions as an alkyating agent; the drug also possesses potent immunosuppressive activity. The serum half-life after IV administration ranges from 3-12 hours; the drug and/or its metabolites can be detected in the serum for up to 72 hours after administration. Following reconstitution as directed with sterile water for injection, cyclophosphamide can be stable for 24 hours at room temperature or 6 days when kept at 2-8° C. Cyclophosphamide can be diluted in 250 ml D5W and infused over one hour. The dose will be based on a subject's body weight. If a subject is obese (BMI>35) drug dosage will be calculated using practical weight as described in. In some cases, an immunosuppressive agent such as cyclophosphamide can be administered from about 1 mg/kg to about 3 mg/kg, from about 3 mg/kg to about 5 mg/kg, from about 5 mg/kg to about 10 mg/kg, from about 10 mg/kg to about 20 mg/kg, 20 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, from about 90 mg/kg to about 100 mg/kg. In some cases, an immunosuppressive agent such as cyclophosphamide is administered in excess of 50 mg/kg of a subject. In some cases, an immunosuppressive agent such as cyclophosphamide can be administered from about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, up to about 100 mg/kg of a subject. In some cases, an immunosuppressive agent such as cyclophosphamide can be administered over at least about 1 day to about 3 days, from 3 days to 5 days, from 5 days to 7 days, from 7 days to about 10 days, from 10 days to 14 days, from 14 days to about 20 days. In some cases, cyclophosphamide can be at a dose of about 60 mg/kg and is diluted in 250 ml 5% dextrose in water and infused over one hour.

An immunosuppressive agent can be, for example, a regime of cyclophosphamide and fludarabine. For example, a cyclophosphamide fludarabine regimen can be administered to a subject receiving an engineered cellular therapy. A cyclophosphamide fludarabine regimen can be administered at a regime of 60 mg/kg qd for 2 days and 25 mg/m² qd for 5 days. A chemotherapeutic regime, for example, cyclophosphamide fludarabine, can be administered from 1 hour to 14 days preceding administration of engineered cells of the present invention. A chemotherapy regime can be administered at different doses. For example, a subject may receive a higher initial dose followed by a lower dose. A subject may receive a lower initial dose followed by a higher dose.

In some cases, an immunosuppressive agent can be an antibody. An antibody can be administered at a therapeutically effective dose. An antibody can be a polyclonal antibody or a monoclonal antibody. A polyclonal antibody that can be administered can be an antilymphocyte or antithymocyte antigen. A monoclonal antibody can be an anti-IL-2 receptor antibody, an anti-CD25 antibody, or an anti-CD3 antibody. An anti-CD20 antibody can also be used. B-cell ablative therapy such as agents that react with CD20, e.g., Rituxan can also be used as immunosuppressive agents.

An immunosuppressive can also be an anti-immunophilin. Anti-immunophilins can be ciclosporin, tacrolimus, everolimus, or sirolimus. Additional immunosuppressive agents can be interferons such as IFN-beta, opiods, anti-TNF binding agents, mycophenolate, or fingolimod.

In some embodiments, a method can further comprise administering radiotherapy to a subject. Radiotherapy can include radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

In some embodiments, a method provided herein can further comprise administering a chemotherapeutic. A chemotherapeutic agent or compound can be a chemical compound useful in the treatment of cancer. Exemplary chemotherapeutic agents that can be used in combination with the disclosed methods include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

In some embodiments, a method can further comprise administering an antiviral to a subject. In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. Additional anti-viral agents that can be administered include but are not limited to anti-Hepatitis B virus (HBV), anti-hepatitis C virus (HCV), anti-human papillomavirus (HPV), and anti-Epstein-Barr virus (EBV).

In some embodiments, a method can further comprise administering an antibiotic to a subject. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3^{rd} or 4^{th} generation, can be cephalosporin or a quinolone. An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin. In some cases, an antibiotic can be 1^{st} generation, 2^{nd} generation, 3^{rd} generation, 4th generation, or 5^{th} generation. A first generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3^{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole. In some cases, an antibiotic can be a bacterial wall targeting agent, a cell membrane targeting agent, a bacterial enzyme interfering agent, a bactericidal agent, a protein synthesis inhibitor, or a bacteriostatic agent. A bacterial wall targeting agent can be a penicillin derivatives (penams), cephalosporins (cephems), monobactams, and carbapenems. β-Lactam antibiotics are bactericidal or bacteriostatic and act by inhibiting the synthesis of the peptidoglycan layer of bacterial cell walls. In some cases an antibiotic may be a protein synthesis inhibitor. A protein synthesis inhibitor can be ampicillin which acts as an irreversible inhibitor of the enzyme transpeptidase, which is needed by bacteria to make the cell wall. It inhibits the third and final stage of bacterial cell wall synthesis in binary fission, which ultimately leads to cell lysis; therefore, ampicillin is usually bacteriolytic. In some cases, a bactericidal agent can be cephalosporin or quinolone. In other cases, a bacteriostatic agent is trimethoprim, sulfamethoxazole, or pentamidine.

In some cases, an agent for the prevention of PCP pneumonia may be administered. For example, Trimethoprim and Sulfamethoxazole can be administered to prevent pneumonia. A dose of trimethoprim and sulfamethoxazole (TMP/SMX; an exemplary sulfa drug) can be 1 tablet PO daily three times a week, on non-consecutive days, on or after the first dose of chemotherapy and continuing for at least about 6 months and until a CD4 count is greater than 200 on at least 2 consecutive lab studies. In some cases, trimethoprim can be administered at 160 mg. Trimethoprim can be administered from about 100 to about 300 mgs. Trimethoprim can be administered from about 100 mg, 125 mg, 150 mg, 175 mg, 200 mg, 225 mg, 250 mg, 275 mg, or up to about 300 mg. In some cases, sulfamethoxazole is administered at 800 mg. Sulfamethoxazole can be administered from about 500 mg to about 1000 mg. Sulfamethoxazole can be administered from about 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or up to about 1000 mgs. In some cases, a TMP/SMX regime can be administered at a therapeutically effective amount. TMP/SMX can be administered from about 1× to about 10× daily. TMP/SMX can be administered 1×, 2×, 3×, 4×, 5×, 6×, 7×, 8×, 9×, 10×, 11×, 12×, 13×, 14×, 15×, 16×, 17×, 18×, 19×, or up to about 20× daily. In some cases, TMP/SMX can be administered on a weekly basis. For example, TMP/SMX can be administered from 1×, 2×, 3×, 4×, 5×, 6×, or up to about 7× a week. A TMP/SMX regime can be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of a cellular therapy, such as FAST-CART.

In some embodiments, the provided methods herein can be used in combination with an anti-angiogenic agent. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis that can be utilized with the provided methods and compositions include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In some embodiments, a method can comprise administration of an additional therapy such as antifungal therapy. In some cases, an anti-fungal is administered to a subject receiving an administration of a composition comprising engineered cells. Antifungals can be drugs that can kill or prevent the growth of fungi. Targets of antifungal agents can include sterol biosynthesis, DNA biosynthesis, and R-glucan biosynthesis. Antifungals can also be folate synthesis inhibitors or nucleic acid cross-linking agents. A folate synthesis inhibitor can be a sulpha based drug. For example, a folate synthesis inhibitor can be an agent that inhibits a fungal synthesis of folate or a competitive inhibitor. A sulpha based drug, or folate synthesis inhibitor, can be methotrexate or sulfamethoxazole. In some cases, an antifungal can be a nucleic acid cross-linking agent. A cross-linking agent may inhibit a DNA or RNA process in fungi. For example, a cross-linking agent can be 5-fluorocytosine, which can be a fluorinated analog of cytosine. 5-fluorocytosine can inhibit both DNA and RNA synthesis via intracytoplasmic conversion to 5-fluorouracil. Other anti-fungal agents can be griseofulvin. Griseofulvin is an antifungal antibiotic produced by Penicillium griseofulvum. Griseofulvin inhibits mitosis in fungi and can be considered a cross linking agent. Additional cross linking agent can be allylamines (naftifine and terbinafine) inhibit ergosterol synthesis at the level of squalene epoxidase; one morpholene derivative (amorolfine) inhibits at a subsequent step in the ergosterol pathway.

In some embodiments, an antifungal agent can be from a class of polyene, azole, allylamine, or echinocandin. In some embodiments, a polyene antifungal is amphotericin B, candicidin, filipin, hamycin, natamycin, nystatin, or rimocidin. In some cases, an antifungal can be from an azole family. Azole antifungals can inhibit lanosterol 14 α-demethylase. An azole antifungal can be an imidazole such as bifonazole, butoconazole, clotrimazole, econazole, fenticonazole, isoconazole, ketoconazole, luliconazole, miconazole, omoconazole, oxiconazole, sertaconazole, sulcoazole, or tioconazole. An azole antifungal can be a triazole such as albaconazole, efinaconazole, epoxiconazole, fluconazole, isavuvonazole, itraconazole, posaconazole, propiconazole, ravuconazole, terconazole, or voriconazole. In some cases an azole can be a thiazole such as abafungin. An antifungal can be an allylamine such as amorolfin, butenafine, naftifine, or terbinafine. An antifungal can also be an echinocandin such as anidulafungin, caspofungin, or micafungin. Additional agents that can be antifungals can be aurones, benzoic acid, ciclopirox, flucytosine, griseofulvin, haloprogin, tolnaftate, undecylenic acid, crystal violet or balsam of Peru. A person of skill in the art can appropriately determine which known antifungal medication to apply based on the fungus infecting the individual. In some cases, a subject will receive fluconazole in combination with engineered cells. An anti-fungal therapy can be administered prophylactically.

In some aspects a treated subject can be monitored post administration with a composition generated by the methods provided herein. In some embodiments, peripheral blood can be obtained from a subject after an administration of engineered cells. In some embodiments, blood serum can be isolated from the peripheral blood of a subject after an administration of engineered cells. In some embodiments, a spinal tap sample can be collected from a subject after an administration of engineered cells. In some embodiments, engineered immune cells from a sample of a treated subject can be quantified from the sample. In some aspects, a sample from a subject that has undergone an administration of engineered cells can be peripheral blood. In some embodiments, engineered cells can be monitored by quantitative PCR (qPCR). A qPCR assay of adoptively transplanted cells can indicate a level of engineered cells that exist in a subject after administration. In some cases, adoptively transferred cells can be monitored using flow cytometry. For example a flow cytometry assay may determine a level of 4-1BB vs TCR. In some cases, a single-cell TCR PCR can be performed. Levels of adoptively transferred cells can be identified on day 7 post infusion. Levels of adoptively transferred cells, such as modified cells, can be identified any of days: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175,180, 185, 190, 195, or up to day 200 post infusion.

In some embodiments, a level of a growth factor in a subject that has been administered engineered cells is quantified. Determining a level of a growth factor in a subject may indicate the subject's reaction to the administered engineered cells. In some aspects, quantifying a level of a growth factor is done to monitor the subject's tolerance to adoptively transferred cells. In some aspects, quantifying a level of a growth factor can indicate that intervention is necessary to prevent, stabilize, or top toxicity. In some aspects, toxicity can be cytokine release syndrome. In some embodiments, a growth factor that can be quantified and/or monitored from a sample of a subject is selected from the group consisting of IL-10, IL-6, tumor necrosis factor α (TNF-α), IL-1β, IL-2, IL-4, IL-8, IL-12, and/or IFN-γ.

In some aspects, an administration of a population of cells comprising engineered cells is repeated. In some aspects, a subject may undergo from about 1, 2, 3, 4, 5, 6, 7, 8, 9, or up to 10 infusions of a population of cells comprising engineered cells. In some aspects, engineered cells are allogeneic to a subject receiving an administration. In some aspects, engineered cells are autologous to a subject receiving the administration.

In some embodiments, methods provided herein can be utilized for the treatment of a disease. In some aspects, methods provided herein can be utilized for the treatment of cancer by targeting the cancer with a population of engineered immune cells. In some embodiments, a subject that is administered the subject engineered cells has cancer. In some aspects, the cancer is a target and is hematological. In some embodiments, a hematological cancer comprises leukemia, myeloma, lymphoma, and/or a combination thereof. In some aspects, leukemia can be chronic lymphocytic leukemia (CLL), T-cell acute lymphoblastic leukemia (T-ALL), acute myeloid leukemia (AML), B cell acute lymphoblastic leukemia (B-ALL), and/or acute lymphoblastic leukemia (ALL). In some embodiments, lymphoma can be mantle cell lymphoma (MCL), T cell lymphoma, Hodgkin's lymphoma, and/or non-Hodgkin's lymphoma. In some aspects, the cancer is a target and is solid. In some embodiments, a solid cancer target or a liquid cancer target is selected from the group comprising: nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, or bladder cancer. Non-limiting examples of cancer include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen. In some aspects, a subject can have minimal residual disease (MRD) after a therapy or administration. MRD can include any of the aforementioned cancers or cancer cells. In some embodiments, MRD is acute lymphoblastic leukemia. In an aspect, a cancer provided herein can express a chemokine such as SDF-1. In an aspect, a cell of a tumor microenvironment of a cancer provided herein expresses a chemokine. A chemokine can attract an immune cell, such as an engineered immune cell provided herein. In an aspect, an engineered immune cell, such as F-CART, can migrate towards a cancer or tumor microenvironment that is high in expression of a chemokine, such as SDF-1. In an aspect, a cancer that expresses a chemokine, such as SDF-1, can be treated with an engineered immune cell provided herein.

In some embodiments, a subject has a BCR-ABL mutation. In some embodiments a BCR-ABL mutation is in a BCR-ABL kinase domain or a portion thereof. In some embodiments, a subject has a T315I and/or V299L mutation in the BCR-ABL kinase domain or portion thereof. In some cases, a subject shows resistance to a tyrosine kinase inhibitor.

In some embodiments, a subject has received a prior treatment. For example, a subject may have received a first line of therapy for a disease such as cancer. In some aspects, a subject may be resistant to a first line of therapy and/or is susceptible of having a tumor after a first line of therapy such as chemotherapy. In some aspects, a subject was pre-treated with chemotherapy prior to an administration of the subject engineered cells.

Provided herein can be methods for administering a therapeutic regime to a subject having a condition such as cancer. In some instances, a cellular composition (for example, comprising a pharmaceutiacl composition comprising engineered immune cells) can be provided in a unit dosage form. A cellular composition can be resuspended in solution and administered as an infusion. Provided herein can also be a treatment regime that includes immunostimulants, immunosuppressants, antibiotics, antifungals, antiemetics, chemotherapeutics, radiotherapy, and any combination thereof. A treatment regime that includes any of the above can be lyophilized and reconstituted in an aqueous solution (e.g., saline solution). In some instances, a treatment is administered by a route selected from subcutaneous injection, intramuscular injection, intradermal injection, percutaneous administration, intravenous (“i.v.”) administration, intranasal administration, intralymphatic injection, and oral administration. In some instances, a subject is infused with a cellular composition comprising engineered cells by an intralymphatic microcatheter.

Drugs used in conjunction with a cell therapy of the present disclosure can be administered orally as liquids, capsules, tablets, or chewable tablets. Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous administration of drugs used in conjunction with a cell therapy of the present disclosure, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. In some cases, cells or therapeutic regimes are administered as infusions. An infusion can take place over a period of time. For example, an infusion can be an administration of a cell or therapeutic regime over a period of about 5 minutes to about 5 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, or up to about 5 hours.

In some embodiments, intravenous administration is used to deliver a precise dose quickly and in a well-controlled manner throughout the body. It is also used for irritating solutions, which would cause pain and damage tissues if given by subcutaneous or intramuscular injection. An intravenous injection can be more difficult to administer than a subcutaneous or intramuscular injection because inserting a needle or catheter into a vein may be difficult, especially if the person is obese. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

Inhalable drugs used in conjunction with a cell therapy of the present disclosure can be administered through the mouth as being atomized into smaller droplets than those administered by the nasal route. That way the drugs can pass through the windpipe (trachea) and into the lungs. Smaller droplets may go deeper into the throat, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In some cases, a treatment regime may be dosed according to a body weight of a subject. In subjects who are determined obese (BMI>35) a practical weight may need to be utilized. BMI is calculated by: BMI=weight (kg)/[height (m)]². An ideal body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases a body surface area may be utilized to calculate a dosage. A body surface area (BSA) may be calculated by: BSA (m2)=√Height (cm)*Weight (kg)/3600.

In some cases, a pharmaceutical composition comprising a cellular therapy can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

In some cases, a therapeutic regime can be administered along with a carrier or excipient. Exemplary carriers and excipients can include dextrose, sodium chloride, sucrose, lactose, cellulose, xylitol, sorbitol, malitol, gelatin, PEG, PVP, and any combination thereof. In some cases, an excipient such as dextrose or sodium chloride can be at a percent from about 0.5%, 1, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%.

Described herein is a method of treating a disease (e.g., cancer) in a recipient comprising transplanting to the recipient one or more cells (including organs and/or tissues) comprising engineered immune cells. Cells prepared by the provided methods can be used to treat cancer. In some cases, a level of disease can be determined in sequence or concurrent with a treatment regime or cellular administration. A level of disease on target lesions can be measured as a Complete Response (CR): Disappearance of all target lesions, Partial Response (PR): At least a 30% decrease in the sum of the longest diameter (LD) of target lesions taking as reference the baseline sum LD, Progression (PD): At least a 20% increase in the sum of LD of target lesions taking as reference the smallest sum LD recorded since the treatment started or the appearance of one or more new lesions, Stable Disease (SD): Neither sufficient shrinkage to qualify for PR nor sufficient increase to qualify for PD taking as references the smallest sum LD. In other cases, a non-target lesion can be measured. A level of disease of a non-target lesion can be Complete Response (CR): Disappearance of all non-target lesions and normalization of tumor marker level, Non-Complete Response: Persistence of one or more non-target lesions, Progression (PD): Appearance of one or more new lesions. Unequivocal progression of existing non-target lesions. In some cases, a subject that undergoes a treatment regime and cellular administration can be evaluated for best overall response. A best overall response can be the best response recorded from the start of treatment until disease progression/recurrence (taking as reference for progressive disease the smallest measurements recorded since the treatment started). A subject's best response assignment can depend on the achievement of both measurement and confirmation criteria. The time to progression can be measured from the date of randomization. In an aspect, a response can refer to monitoring a cancer burden or tumor burden in a subject. In an aspect, a reduced cancer burden is observed in a subject when the subject is administered a population comprising engineered immune cells, such as F-CART, as compared to the cancer burden observed in a comparable subject administered a comparable population that undergoes an ex vivo expansion for 2 or more weeks, such as C-CART. In an aspect, cancer burden is reduced by at least 10%, 20%, 30%, 40%, 50%, 60%, 70%, 80%, 90%, 95%, or 99% in a subject treated with a population comprising engineered immune cells, such as F-CART, as compared to a comparable subject administered a comparable population that undergoes an ex vivo expansion for 2 or more weeks, such as C-CART. In an aspect, a subject as described herein can be a mammal. A mammal can be a human, dog, horse, pig, mouse, rat, or monkey.

To be assigned a status of PR or CR, changes in tumor measurements must be confirmed by repeat studies that should be performed at least about 4 weeks after the criteria for response are first met. In the case of SD, follow-up measurements must have met the SD criteria at least once after study entry at a minimum interval of 6-8 weeks. In some cases, a duration of overall response can be measured from the time measurement criteria are met for CR/PR (whichever is first recorded) until the first date that recurrent or progressive disease is objectively documented (taking as reference for progressive disease the smallest measurements recorded since the treatment started). The duration of overall complete response can be measured from the time measurement criteria are first met for CR until the first date that recurrent disease is objectively documented. Stable disease can be measured from the start of the treatment until the criteria for progression are met, taking as reference the smallest measurements recorded since the treatment started. In some cases, measurable disease can be taken and recorded in metric notation using a ruler or calipers. All baseline evaluations can be performed as closely as possible to the beginning of treatment. A lesion can be considered measurable when it is superficial (e.g., skin nodule and palpable lymph nodes) and over at least about 10 mm in diameter using calipers. In some cases, color photography can be taken.

In other cases, a computerized tomography scan (CT) can or magnetic resonance imaging (MRI) can be taken. A CT can be taken on a slice thickness of 5 mm or less. If CT scans have slice thickness greater than 5 mm, the minimum size for a measurable lesion should be twice the slice thickness. In some cases, an FDG-PET scan can be used. FDG-PET can be used to evaluate new lesions. A negative FDG-PET at baseline, with a positive FDG-PET at follow up is a sign of progressive disease (PD) based on a new lesion. No FDG-PET at baseline and a positive FDG-PET at follow up: if a positive FDG-PET at follow-up corresponds to a new site of disease confirmed by CT, this is PD. If a positive PDG-PET at follow up corresponds to a pre-existing site of disease on CT that may not be progressing on a basis of anatomic imagines, this may not be PD. In some cases, FDG-PET may be used to upgrade a response to a CR in a manner similar to biopsy in cases where a residual radiographic abnormality is thought to represent fibrosis or scarring. A positive FDG-PET scan lesion means one which is FDG avid with an uptake greater than twice that of the surrounding tissue on an attenuation corrected image.

In some cases an evaluation of a lesion can be performed. A complete response (CR) can be a disappearance of all target lesions. Any pathological lymph nodes (target or non-target) may have reduction in short axis to less than 10 mm. A partial response (PR) can be at least a 30% decrease in a sum of the diameters of target lesions, taking as reference the baseline sum of diameters. Progressive disease (PD) can be at least a 20% increase in the sum of the diameters of target lesions, taking as reference the smallest sum. In addition to the relative increase of 20%, the sum must also demonstrate an absolute increase of at least 5 mm. Stable disease (SD) can be neither sufficient shrinkage to quality for PR nor sufficient increase to quality for PD, taking as reference the smallest sum of diameters.

In some cases, non-target lesions can be evaluated. A complete response of a non-target lesion can be a disappearance and normalization of tumor marker level. All lymph nodes must be non-pathological in size (less than 10 mm short axis). If tumor markers are initially above the upper normal limit, they must normalize for a patient to be considered a complete clinical response. Non-CR/Non-PD is persistence of one or more non-target lesions and or maintenance of tumor marker level above the normal limit. Progressive disease can be appearance of one or more new lesions and or unequivocal progression of existing non-target lesions. Unequivocal progression should not normally trump target lesion status. In some cases, a best overall response can be the best response recorded from the start of treatment until disease progression/recurrence.

In some cases, a subject treated with a treatment regime or cellular product described herein can experience an adverse event associated with the regime or cellular product. An adverse event can be any reaction, side effect, or untoward event that occurs during the course of the treatment associated with the use of a drug in humans, whether or not the event is considered related to the treatment or clinically significant. In some cases, an adverse event can include events reported by a subject, as well as clinically significant abnormal findings on physical examination or laboratory evaluation. A new illness, symptom, sign or clinically significant laboratory abnormality or worsening of a pre-existing condition or abnormality can be considered an adverse event. All adverse events, including clinically significant abnormal findings on laboratory evaluations, regardless of severity, will be followed until resolution to grade 2 or less with the exception of lymphopenia and alopecia. If an adverse event is not expected to resolve to grade 2 or less a subject may cease therapy. In some cases, a treatment regime may be administered with toxicity reducing agents. A toxicity reducing agent can be a fever or vomit reducing agent. For example Mesna can be administered to reduce toxicities such as nausea, vomiting, and diarrhea.

In an aspect, a population of cells can undergo pre-infusion testing prior to an administration or concurrent with an administration. Pre-infusion or pre-administration testing can be performed to ensure an engineered cellular product is functional, sterile, and capable of functioning post-infusion. Pre-infusion testing can comprise determining a phenotype, cytotoxicity, memory/stemness, exhaustion, bone marrow migration, ELISA, and any combination thereof. In an aspect, a pre-administration testing can comprise performing an in vitro or an vivo assay. In an aspect, a level of cytotoxicity may be determined in a population of engineered cells. For example, a population of cells can be evaluated by FACs for expression of any one of: CD3, CD4, CD8, CD45RO, CCR7, CD45RA, CD62L (L-selectin), CD27, CD28, and IL-7Rα, CD95, IL-2Rβ, CXCR3, and LFA-1. In an aspect, functional testing can also comprise a co-culture assay, cytotoxicity assay, ELISA (for example to quantify interleukin-2 (IL-2), and/or IFN-γ section), or ELISPOT assays. In an aspect, a population provided herein is further characterized in that a greater proliferation, cytotoxicity, and/or bone marrow migration is observed in the population as compared to the proliferation, cytotoxicity, and/or bone marrow migration of a comparable population that undergoes an ex vivo expansion over 2 weeks or a comparable population that is: (a) absent a concurrent activation of a population of cells with an activation moiety and (b) an introduction of a polynucleotide encoding for a CAR. In an aspect, a population provided herein is further characterized in that it comprises a greater memory and/or stemness as compared to a comparable population that undergoes an ex vivo expansion over 2 weeks or a comparable population that is (a) absent a concurrent activation of a population of cells with an activation moiety and (b) an introduction of a polynucleotide encoding for a CAR. In an aspect, a level of proliferation, memory/stemness, cytotoxicity (killing capacity), and/or BM migration can be or can be about 10%, 20%, 30%, 40%, 50%, 60%, 70%, 80%, 90%, 95%, or up to about 100% greater than a comparable population of cells that undergoes an ex vivo expansion over 2 weeks or that is (a) absent a concurrent activation of a population of cells with an activation moiety and (b) an introduction of a polynucleotide encoding for a CAR.

In some embodiments, provided herein is a point-of-care facility. A point-of-care facility can be a hospital, laboratory, clinic, vehicle, medical center, recreational vehicle, a home, to name a few exemplary facilities. A point-of-care facility can comprise cell infusion equipment. Cell infusion equipment can comprise any one of: a bag, pump, syringe, tubing, a lumen, bioreactor, incubator, hemocytometer, centrifuge, thermometer, needle, suction machine, oxygen tank, VAD lumen, and any combination thereof. In some embodiments, a point-of-care facility comprises cell infusion equipment. Cell infusion equipment can be configured to: infuse a population of cells that comprises engineered immune cells that have not been subject to ex-vivo expansion for 2 or more weeks. In an aspect, the population of immune cells is a pharmaceutical composition. In some aspects, cell infusion equipment comprises a population of immune cells is can be further characterized in that: cell memory T cells (TCM) in the population are more abundant than effector memory T cells (TEM). In some aspects, cell infusion equipment comprises a population of immune cells wherein at least 2% of the population are stem memory T cells (TSCM). In some embodiments, a point-of-care facility comprises a cell processing equipment configured to (a) receive a population of cells comprising immune cells from a subject and (b) activate the population of immune cells with an activation moiety, and concurrently, introduce a polynucleotide encoding for at least a chimeric antigen receptor (CAR) to said immune cells. In some aspects, the CAR comprises (i) a ligand binding domain specific for a ligand, (ii) a transmembrane domain, and (iii) an intracellular signaling domain. In some aspects, the cell processing equipment is further configured to (c) infuse the population of immune cells of (b) into a subject within 2 weeks or less from the time of performing (b) in some aspects step (c) is performed within 1 week or less from the time of performing (b). In some embodiments, the cell processing equipment of the point-of-care facility is configured to perform step (a) and (b) within 24 hours. In some embodiments, the cell processing equipment of the point-of-care facility is configured to perform step (a) and (b) within 3 hours. In some embodiments, the cell processing equipment of the point-of-care facility is configured to perform step (a) and (b) within 1 hour. In some embodiments, the cell processing equipment of the point-of-care facility is configured to perform step (a) and (b) within 30 minutes.

### EXAMPLES

Various aspects of the disclosure are further illustrated by the following non-limiting examples.

### Example 1: Construction of Lentiviral Vector

Nucleotide sequences including EF1a promoter (as set forth in SEQ ID NO: 1), anti-CD19 scFv (as set forth in SEQ ID NO: 3), CD28 hinge region, CD28 transmembrane region and CD28 costimulatory signal molecule (as set forth in SEQ ID NO: 5), CD3ζ intracellular domain (as set forth in SEQ ID NO: 7), CAR (as set forth in SEQ ID NO: 9) were artificially synthesized.

SEQ ID NO: 1:
gggcagagcg cacatcgccc acagtccccg agaagttggg
gggaggggtc ggcaattgaa cgggtgccta gagaaggtgg
cgcggggtaa actgggaaag tgatgtcgtg tactggctcc
gcctttttcc cgagggtggg ggagaaccgt atataagtgc
agtagtcgcc gtgaacgttc tttttcgcaa cgggtttgcc
gccagaacac agctgaagct tcgaggggct cgcatctctc
cttcacgcgc ccgccgccct acctgaggcc gccatccacg
ccggttgagt cgcgttctgc cgcctcccgc ctgtggtgcc
tcctgaactg cgtccgccgt ctaggtaagt ttaaagctca
ggtcgagacc gggcctttgt ccggcgctcc cttggagcct
acctagactc agccggctct ccacgctttg cctgaccctg
cttgctcaac tctacgtctt tgtttcgttt tctgttctgc
gccgttacag atc
SEQ ID NO: 3:
atgcttctcc tggtgacaag ccttctgctc tgtgagttac
cacacccagc attcctcctg atcccagaca tccagatgac
acagactaca tcctccctgt ctgcctctct gggagacaga
gtcaccatca gttgcagggc aagtcaggac attagtaaat
atttaaattg gtatcagcag aaaccagatg gaactgttaa
actcctgatc taccatacat caagattaca ctcaggagtc
ccatcaaggt tcagtggcag tgggtctgga acagattatt
ctctcaccat tagcaacctg gagcaagaag atattgccac
ttacttttgc caacagggta atacgcttcc gtacacgttc
ggagggggga ctaagttgga aataacaggc tccacctctg
gatccggcaa gcccggatct ggcgagggat ccaccaaggg
cgaggtgaaa ctgcaggagt caggacctgg cctggtggcg
ccctcacaga gcctgtccgt cacatgcact gtctcagggg
tctcattacc cgactatggt gtaagctgga ttcgccagcc
tccacgaaag ggtctggagt ggctgggagt aatatggggt
agtgaaacca catactataa ttcagctctc aaatccagac
tgaccatcat caaggacaac tccaagagcc aagttttctt
aaaaatgaac agtctgcaaa ctgatgacac agccatttac
tactgtgcca aacattatta ctacggtggt agctatgcta
tggactactg gggtcaagga acctcagtca ccgtctcctc
agcggccgca gactacaaag acgatgacga caag
SEQ ID NO: 5:
attgaagtta tgtatcctcc tccttaccta gacaatgaga
agagcaatgg aaccattatc catgtgaaag ggaaacacct
ttgtccaagt cccctatttc ccggaccttc taagcccttt
tgggtgctgg tggtggttgg gggagtcctg gcttgctata
gcttgctagt aacagtggcc tttattattt tctgggtgag
gagtaagagg agcaggctcc tgcacagtga ctacatgaac
atgactcccc gccgccccgg gcccacccgc aagcattacc
agccctatgc cccaccacgc gacttcgcag cctatcgctc
c
SEQ ID NO: 7:
agagtgaagt tcagcaggag cgcagacgcc cccgcgtacc
agcagggcca gaaccagctc tataacgagc tcaatctagg
acgaagagag gagtacgatg ttttggacaa gagacgtggc
cgggaccctg agatgggggg aaagccgaga aggaagaacc
ctcaggaagg cctgtacaat gaactgcaga aagataagat
ggcggaggcc tacagtgaga ttgggatgaa aggcgagcgc
cggaggggca aggggcacga tggcctttac cagggtctca
gtacagccac caaggacacc tacgacgccc ttcacatgca
ggccctgccc cctcgc
SEQ ID NO: 9:
atgcttctcc tggtgacaag ccttctgctc tgtgagttac
cacacccagc attcctcctg atcccagaca tccagatgac
acagactaca tcctccctgt ctgcctctct gggagacaga
gtcaccatca gttgcagggc aagtcaggac attagtaaat
atttaaattg gtatcagcag aaaccagatg gaactgttaa
actcctgatc taccatacat caagattaca ctcaggagtc
ccatcaaggt tcagtggcag tgggtctgga acagattatt
ctctcaccat tagcaacctg gagcaagaag atattgccac
ttacttttgc caacagggta atacgcttcc gtacacgttc
ggagggggga ctaagttgga aataacaggc tccacctctg
gatccggcaa gcccggatct ggcgagggat ccaccaaggg
cgaggtgaaa ctgcaggagt caggacctgg cctggtggcg
ccctcacaga gcctgtccgt cacatgcact gtctcagggg
tctcattacc cgactatggt gtaagctgga ttcgccagcc
tccacgaaag ggtctggagt ggctgggagt aatatggggt
agtgaaacca catactataa ttcagctctc aaatccagac
tgaccatcat caaggacaac tccaagagcc aagttttctt
aaaaatgaac agtctgcaaa ctgatgacac agccatttac
tactgtgcca aacattatta ctacggtggt agctatgcta
tggactactg gggtcaagga acctcagtca ccgtctcctc
agcggccgca gactacaaag acgatgacga caagattgaa
gttatgtatc ctcctcctta cctagacaat gagaagagca
atggaaccat tatccatgtg aaagggaaac acctttgtcc
aagtccccta tttcccggac cttctaagcc cttttgggtg
ctggtggtgg ttgggggagt cctggcttgc tatagcttgc
tagtaacagt ggcctttatt attttctggg tgaggagtaa
gaggagcagg ctcctgcaca gtgactacat gaacatgact
ccccgccgcc ccgggcccac ccgcaagcat taccagccct
atgccccacc acgcgacttc gcagcctatc gctccagagt
gaagttcagc aggagcgcag acgcccccgc gtaccagcag
ggccagaacc agctctataa cgagctcaat ctaggacgaa
gagaggagta cgatgttttg gacaagagac gtggccggga
ccctgagatg gggggaaagc cgagaaggaa gaaccctcag
gaaggcctgt acaatgaact gcagaaagat aagatggcgg
aggcctacag tgagattggg atgaaaggcg agcgccggag
gggcaagggg cacgatggcc tttaccaggg tctcagtaca
gccaccaagg acacctacga cgcccttcac atgcaggccc
tgccccctcg ctaa

The nucleic acid sequence encoding the CAR was cloned into the lentiviral vector FUGW, plasmid named GCP042. 293. T cells were simultaneously transfected with plasmid GCP042 and other packaging plasmids (helper plasmids). Briefly, 2×10⁶ 293T cells were seeded in a 150 cm² culture dish at a density of 1.3×10⁴ cells/cm² in DMEM medium containing 10% FBS. The cells were then cultured at 37° C., 5% CO₂ and saturated humidity for 3 days before transfection.

18 pg helper plasmids and 24 pg GCP042 were added to a centrifuge tube containing 3 mL DMEM medium, and then 126 mg PEI was added to the tube to obtain a mixture. The mixture was allowed to stand at room temperature for 30 minutes and then supplemented with 12 mL DMEM containing 2% FEB to obtain the transfection medium. For transfection, after removing the culture medium, 293 T cells were incubated with the transfection medium for 4 hours and then the transfection medium was replaced with 20 mL DMEM medium containing 2% FBS. 72 hours later, the medium was collected and centrifuged at 3000 g, 4° C. for 10 min. Then the supernatant was filtered with a 0.45 μm filter for further purification. The filtrate was further subjected to centrifuge at 27000 g, 4° C. for 2 hours. The pellet was collected and re-suspended with 100 μL pre-chilled PBS to obtain the GCL042 lentivirus suspension, and kept at 4° C. overnight. The next day, the virus suspension was aliquoted for further use.

### Example 2: Preparation of F-CART Cells

To prepare F-CART cells, 100 mL peripheral blood was collected from a healthy donor. PBMCs were isolated by density gradient centrifugation at 500-600 g for 20-30 minutes. Magnetic beads coupled with CD28 antibody and CD3 antibody (CD3/CD28 Dynabeads, purchased from ThermoFisher) was used to sort and enrich T cells. The T cells were then transfected with GCL042 as prepared in Example 1 in X-vivo15 medium at 37° C. at a cell density between 0.1×10⁶ cells/mL to 10×10⁶ cells/mL. After the transfection, without further expansion, the CAR-T cells of the present disclosure were obtained (also named F-CART or F-CAR-T cells herein). In this procedure, cells were not activated before transfection, and these cells are also referred as F-CART-FV1 cells in the present disclosure.

Similarly, about 100 mL peripheral blood was collected from a healthy donor, and PBMCs were isolated by density gradient centrifuge at 500-600 g for 20-30 minutes. Magnetic beads coupled with CD28 antibody and CD3 antibody (CD3/CD28 Dynabeads, purchased from ThermoFisher) was used to sort and enrich T cells. T cells were further incubated with the CD3/CD28 Dynabeads at a ratio from 0.1:1 to 5:1 (CD3/CD28 Dynabeads: T cells), together with 300 IU/mL IL2 to activate the cells. Meanwhile or subsequently, the T cells were transfected with GCL042 at a cell density of 0.1-1×10⁶ cells/mL at 37° C. for 4 hours, and then washed with saline buffer. Without further expansion, the F-CART cells of the present disclosure were obtained. In this procedure, cells were activated, and these cells are also referred as F-CART-FV2 cells in the present disclosure.

Table 1 summarized the preparation processes of F-CART-FV1 cells versus F-CART-FV2 cells

TABLE 1
Preparation processes of F-CART-FV1 and F-CART-FV2 cells

| Preparation | T cell status | Process |
|---|---|---|
| F-CART-FV1 | Resting | Virus was added directly for |
| | | transfection |
| F-CART-FV2 | Resting - activation | Virus was added directly for |
| | | transfection |

### Example 3: MOI of Lentivirus and the Ratio of CAR Positive Cells

The ratios of CAR positive cells by using FV1 preparation process and FV2 preparation process were subsequently compared. Briefly, about 100 mL peripheral blood was collected from a healthy donor, and PBMCs were isolated by density gradient centrifuge at 500-600 g for 20-30 minutes. Then CD3/CD28 Dynabeads was used to sort and enrich T cells.

1×10⁷ sorted T cells were divided into two groups, and F-CART-FV1 and F-CART-FV2 cells were prepared as described in Example 2, respectively. The GCL042 was used to transfect the T cells at a cell density of 2×10⁶ cells/mL with an MOI between 3 and 4. In particular, the amount of the virus used was calculated as: number of virus=cell number×MOI/titer of the virus. Then the virus as calculated was incubated with T cells for 16-24 hours to infect the T cells. After transfection, the virus was removed by centrifuge and then the cells were collected and cultured for 3-5 more days. 0.5×10⁶-1.0×10⁶ cells from each group were collected and subject to flow cytometry for CAR positive ratio analysis. The result is as shown in Table 2.

TABLE 2
CAR positive ratios by different preparation processes

| Preparation | MOI | Positive ratio (%) |
|---|---|---|
| F-CART-FV1 | 3.2 | 6.2% |
| F-CART-FV2 | 3.0 | 33.1% |

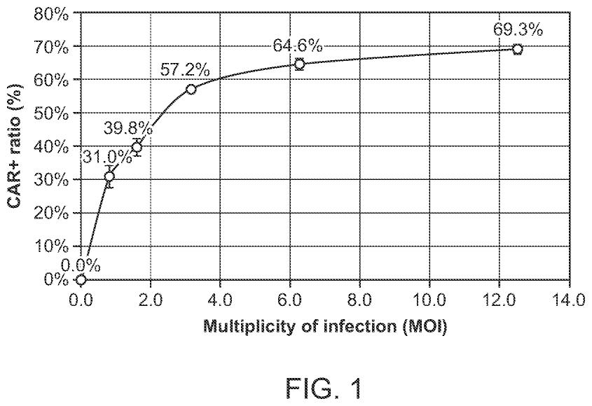

From Table 2, it can be seen that the CAR positive ratio in F-CART-FV2 preparation is higher than that in F-CART-FV1 preparation. Subsequently, the correspondence between MOI and CAR positive ratio was studied by using F-CART-FV2 cells, and the result is as shown in FIG. 1. It can be seen from FIG. 1 that the CAR positive ratios were 31.0% and 39.8%, respectively, by using an MOI of 0.8 and 1.6, indicating that positive ratio was also affected by additional factors other than MOI at this stage; the CAR positive ratios were 39.8% and 57.2%, respectively, by using MOI of 1.6 and 3.2, indicating that as the amount of virus increased, the positive ratio showed a nearly 1.5-fold change, and the positive ratio was basically correlated to the amount of virus used at this stage; the CAR positive ratios were 57.2%, 64.6% and 69.3%, respectively, by using an MOI of 3.2, 6.4 and 12.8, indicating that the positive ratio did not significantly increase with the amount of virus, and the virus infection entered into a platform at this stage. This result suggests that the preferred MOI for preparing F-CART of the present disclosure, especially F-CART-FV2, is between 1.6 and 6.4, by which the positive ratio of the F-CART can be more efficiently and stably controlled.

### Example 4: Preparation of C-CART Cells

Control cells (also named as the second reference cells, C-CART or C-CAR-T cells) were prepared according to the method described by Kochenderfer, J. N et al. (2013), “Donor-derived CD19-targeted T cells cause regression of malignancy persisting after allogeneic hematopoietic stem cell transplantation.” Blood 122(25): 4129-4139.

Briefly, about 100 mL peripheral blood was collected from a healthy donor, and PBMCs were isolated by density gradient centrifuge at 500-600 g for 20-30 minutes. CD3/CD28 Dynabeads was used to sort and enrich T cells. Then the T cells were further incubated with the CD3/CD28 Dynabeads at a ratio from 0.1:1 to 5:1 (CD3/CD28 Dynabeads: T cells), together with 300 IU/mL IL2 to activate the cells. Meanwhile or subsequently, the T cells were transfected with GCL042 at a cell density of 0.1-10×10⁶ cells/mL at 37° C. for 4 hours, and then washed with saline buffer. After the activation and transfection, the modified cells were cultured for 8 days for expansion so that to obtain the control cells (also named as the second reference cells, C-CART or C-CAR-T cells).

### Example 5: Positive Ratio in F-CART Cells Versus C-CART Cells

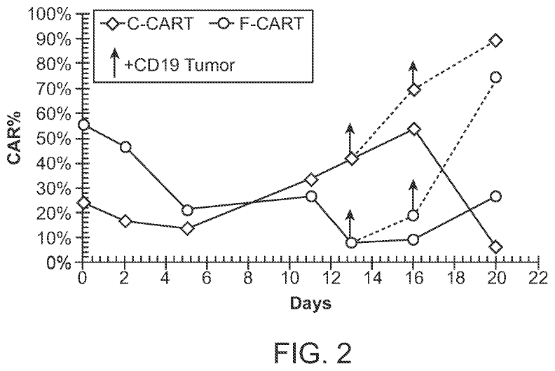

Changes in the ratio of CAR positive cells (CAR+) during in vitro culture in F-CART (such as F-CART-FV2 cells) and C-CART cells were compared after recovery from cryopreservation, in the presence and absence of CD19⁺ tumor cell stimulation. The result is as shown in FIG. 2. It can be seen from FIG. 2 that for the recovered F-CART cells, CAR+ ratio decreased with culture time and after adding tumor antigens, the CAR+ ratio went up again (the solid line in FIG. 2 represents the group without adding tumor antigens, and the dash line represents the group with tumor antigens), indicating that CAR+ cells can be enriched by tumor antigen stimulation, and the polynucleotide encoding CAR was stably incorporated in the genome of T cells. FIG. 2 also shows that, before tumor antigen stimulation, at the starting point, the CAR+ ratio in F-CART cells was much higher than that in C-CART cells, and since day 16, the CAR+ ratio in C-CART cells significantly decreased, but the CAR+ ratio in F-CART still showed slightly increase. After the stimulation by tumor antigen, both F-CART and C-CART cells displayed significantly increased CAR+ ratios.

### Example 6: Analysis of Lymphocyte Subpopulations

Lymphocyte subpopulations were analyzed in the starting material (corresponding T cells without viral transfection) and the F-CART cells (such as F-CART-FV2 cells) by conventional flow cytometry (see Garcia R L et al., Analysis of proliferative grade using anti-PCNA/cyclin monoclonal antibodies in fixed, embedded tissues. Comparison with flow cytometric analysis. The American journal of pathology, 1989, 134(4): 733).

Expression of markers including CD3, CD4, CD8, CD45RO and CD62L was analyzed through flow cytometry by using 2-3×10⁶ of starting T cells and F-CART cells, respectively. The results are as shown in Table 3 and FIG. 3.

TABLE 3
Subpopulations of lymphocytes

| | CD3⁺ | CD4⁺/CD8⁻ | CD8⁺/CD4⁻ | TEM | TCM | T_{N} |
|---|---|---|---|---|---|---|
| Starting | 74.5% | 38.1% | 45.2% | 23.0% | 21.5% | 32.7% |
| Material | | | | | | |
| F-CART | 99.9% | 43.2% | 45.4% | 45.9% | 23.5% | 15.3% |

In Table 3, TEM represents effector T cells having CD45RO⁺/CD62L⁻; TCM represents central memory T cells having CD45RO⁺/CD62L⁺; T_{N} represents initial (or naive) T cells having CD45RO⁻/CD62L⁺ with great differentiation potential, which are able to differentiate into cell subpopulations such as TEM and TCM.

The result shows that the proportion of CD3⁺ T cells in F-CART was much higher than that of the starting material, indicating effective sorting and enrichment of T cells. The proportions of CD4⁺ T and CD8⁺ T cells in F-CART are substantially identical to those of the starting material. The proportions of lymphocyte subpopulations were changed in F-CART compared to the starting material, where the proportions of TCM and TEM in F-CART were increased and the proportion of T_{N} was decreased. This may be due to the differentiation of the original T_{N} population after T cell activation. These results indicate that the F-CART preparation process of the present application can be used to prepare activated T cells with differentiation potential.

### Example 7: In Vitro Expansion of CAR-T Cells

Cell viability and number of living cells were detected on day 0, 2, 5, 8, 11 and 13 in F-CART cells (such as F-CART-FV2 cells) and C-CART cells to compare the in vitro proliferation of F-CART (such as F-CART-FV2) and C-CART preparations.

Briefly, X-vivo15 medium with 300 IU/mL IL-2 was pre-warmed in 37° C. water bath. F-CART cells (such as F-CART-FV2 cells) and C-CART cells were thawed in the 37° C. water bath for 2-3 minutes, and then transferred to the pre-warmed medium, mixed well, and the total volume of the cell suspension was measured. Cell viability and cell density in 300 μL of F-CART and C-CART cell suspension were calculated by a NC-200 counter, and then the cell numbers were calculated based on the volume. Then cells in the two groups were subject to centrifuge at 250-300 g for 8-10 min. Then supernatant was removed, and cells were re-suspended with an appropriate amount of medium to a density of 0.5×10⁶ cells/mL to 1.0×10⁶ cells/mL and then seeded in a plate and cultured at 37° C., 5% CO₂. Cell viability and living cell numbers were calculated on day 0, 2, 5, 8, 11 and 13 to compare the proliferation of F-CART cells versus C-CART cells. The cell density was kept between 0.5×10⁶ cells/mL and 1.0×10⁶ cells/mL during the process.

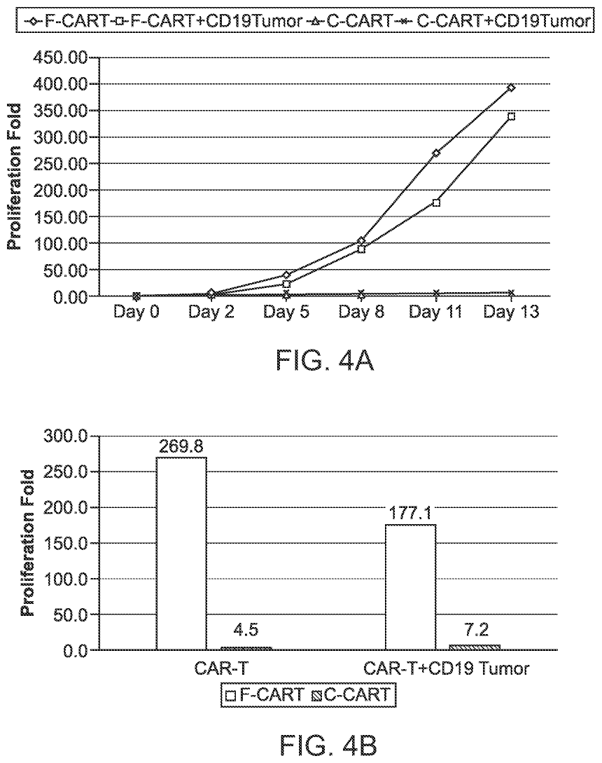

Furthermore, proliferation of the two groups was compared under the stimulation of tumor antigen. Briefly, primary tumor cells expressing CD19 (from B-ALL tumor patient) were co-cultured with F-CART cells and C-CART cells since day 0. Cell viability and living cell numbers were calculated on day 2, 5, 8, 11 and 13, and the cell density was kept between 0.5×10⁶ cells/mL and 1.0×10⁶ cells/mL during the process. The result is as shown in Table 4, FIG. 4A, and FIG. 4B.

TABLE 4
Fold of CAR-T cell proliferation

| | Day 0 | Day 2 | Day 5 | Day 8 | Day 11 | Day 13 |
|---|---|---|---|---|---|---|
| F-CART | 1.00 | 5.04 | 39.62 | 103.97 | 269.84 | 395.35 |
| F-CART + | 1.00 | 3.43 | 24.53 | 90.21 | 177.14 | 339.82 |
| CD19 Tumor | | | | | | |
| C-CART | 1.00 | 1.76 | 2.42 | 2.60 | 4.49 | 5.33 |
| C-CART + | 1.00 | 1.49 | 4.20 | 6.14 | 7.22 | 8.14 |
| CD19 Tumor | | | | | | |

The results show that the F-CART cells had significantly stronger proliferation ability than that of C-CART cells, with or without tumor cell stimulation, indicating the superior expansion capability of the F-CART cells.

### Example 8: In Vitro Tumor Killing Efficacy of CAR-T

To compare the in vitro tumor killing efficacy of F-CART (such as F-CART-FV2) and C-CART preparations, F-CART cells (such as F-CART-FV2 cells) and C-CART cells were thawed and cultured in X-vivo15 medium containing 10% (v/v) AB serum and 300 IU/mL IL-2, and the tumor killing efficacy was detected on day 2.

Briefly, X-vivo15 medium containing 10% (v/v) AB serum and 300 IU/mL IL-2 was pre-warmed in 37° C. water bath. F-CART cells prepared (such as F-CART-FV2 cells) and C-CART cells were thawed in the 37° C. water bath for 2-3 minutes, and then transferred to the pre-warmed medium, mixed well, and the total volume of the cell suspension was measured. Cell viability and cell density in 300 μL of F-CART and C-CART cell suspension were calculated by a NC-200 counter, and then cell numbers were calculated based on the volume. Cells in the two groups were subject to centrifuge at 250-300 g for 8-10 min. Supernatant was removed, and cells were re-suspended with an appropriate amount of X-vivo15 medium containing 10% (v/v) AB serum and 300 IU/mL IL-2 to a density of 0.5×10⁶ cells/mL-1.0×10⁶ cells/mL and then seeded in a plate and cultured at 37° C., 5% CO₂.

On day 2, 0.5×10⁶-1.0×10⁶ cells from each group were subject to CAR+ ratio analysis. As target cells, HELA-CD19, HELA and HEK293T cells under P2-P10 were washed with 10 mL DPBS and digested with 5-10 mL 0.25% trypsin at 37° C. for 1-3 min, and then neutralized with 5-10 mL complete medium (containing 10% FBS) and pipetted repeatedly to form single cell suspension. Subsequently, the three groups of targets cells were subject to centrifuge at 300 g for 5 min. Supernatant was removed, and cells were re-suspended with RPMI 1640 complete medium containing 10% FBS. Cell numbers were counted, and the cell viabilities in all three groups were ≥85%. Then the cell density was adjusted to 1×10⁵ cells/mL.

The detection procedure was established according to the instruction of xCELLLigence. In particular, each group of target cells was added to E-plate view 96 well plate and allowed to stand in an incubator for 30 min, and then attachment of the cells was checked. When Cell-Index of HELA-CD19 cells detected by xCELLLIgence ≥2, based on the CAR+ ratio analyzed above, required number of CAR⁺ cells were collected, and subject to centrifuge at 300 g for 5 min. Supernatant was removed and cells were re-suspended in RPMI 1640 complete medium to an appropriate density. E-plate view 96 plate was removed from xCELLIgence detection and the supernatant was discarded. Based on the required ratio of the CAR-T cells to the target cells, CAR-T cells were added to each well of the plate. Then the E-plate view 96 plate was subject to cell killing detection for 24-48 hours.

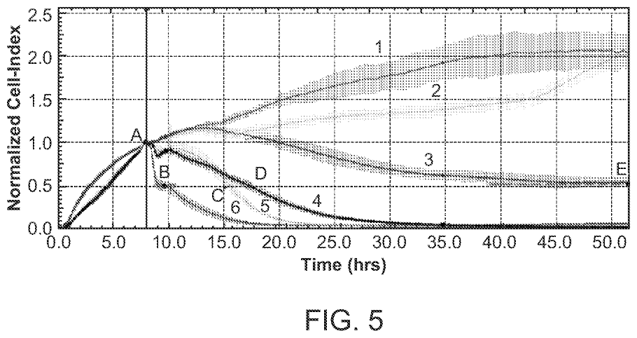

Data was collected and plotted, as shown in Table 5 and FIG. 5, where half killing time of target cells was calculated as the time point having the maximum of Cell-Index (CImax) minuses the time point having half of the maximum Cell-Index (CImax/2).

In FIG. 5, 1 is HELA-CD19, 2 is NT+HELA-CD19 (5:1), 3 is C-CART+HELA-CD19 (1:1), 4 is F-CART+HELA-CD19 (1:1), 5 is C-CART+HELA-CD19 (5:1), 6 is F-CART+HELA-CD19 (5:1). A is the starting point, the time is 7.90 hours, the Cell-Index is 1.02; B is F-CART+HELA-CD19 (5:1), the time is 8.91 hours, the Cell-Index is 0.51; C is C-CART+HELA-CD19 (5:1), the time is 15.23 hours, the Cell-Index is 0.51; D is F-CART+HELA-CD19 (1:1), the time is 16.77 hours, the Cell-Index is 0.51; E is C-CART+HELA-CD19 (1:1), the time is 51.23 hours, the Cell-Index is 0.52.

TABLE 5
Overview of the half killing time

| Half killing | CART + | CART + |
|---|---|---|
| time (h) | HELA − CD19(1:1) | HELA − CD19(5:1) |
| F-CART | 8.87 h | 1.01 h |
| C-CART | 43.33 h | 7.33 h |

The results show that, in the two models where effector cell: target cell was 5:1 and 1:1, the half killing time of F-CART was shorter than that of C-CART, suggesting the killing efficacy of F-CART for tumor cells is significantly stronger than C-CART. Similar results were also observed by using HELA cells and HEK293T cells.

### Example 9: Response of CAR-T to Tumor Antigen Stimulation

Cytokines released by F-CART (such as F-CART-FV2) and C-CART preparations were compared to evaluate the response to tumor antigen stimulation, where F-CART cells (such as F-CART-FV2 cells) and C-CART cells were subject to cytokine detection on the day 2, and the un-transfected T cells (starting material) as described in Example 2 were used as control.

Briefly, culture medium was pre-warmed in 37° C. water bath. F-CART cells (such as F-CART-FV2 cells) and C-CART cells were thawed in the 37° C. water bath for 2-3 minutes, and then transferred to the pre-warmed medium, mixed well, and the total volume of the cell suspension was measured. Cell viability and cell density in 300 μL of F-CART and C-CART cell suspension were calculated by a NC-200 counter, and the cell numbers were calculated based on the volume. Then cells were centrifuges at 250-300 g for 8-10 min. The supernatant was removed, and cells were re-suspended with an appropriate amount of medium to a density of 0.5×10⁶ cells/mL to 1.0×10⁶ cells/mL, and then seeded in a plate and cultured at 37° C., 5% CO₂. On day 2, cells were pipetted and mixed well, and then 0.3×10⁶-1.0×10⁶ cells from each group were subject to CAR+ ratio analysis. Based on the CAR+ ratio, an appropriate amount of cells were collected and subject to centrifuge at 300 g for 5 min, and then the supernatant was removed and cells were re-suspended with RPMI 1640 complete medium to a density of 1×10⁶ cells/mL.

Molt4 cells (without tumor antigen stimulation) and Raji cells (with tumor antigen stimulation) were used as negative and positive targets, respectively. 0.3-0.5 mL target cells suspension was subject to cell number and viability calculation first. Then the two groups of target cells were further subject to centrifuge at 300 g for 5 min. The supernatant was removed and the cells were re-suspended with RPMI 1640 complete medium to a density of 1×10⁶ cells/mL.

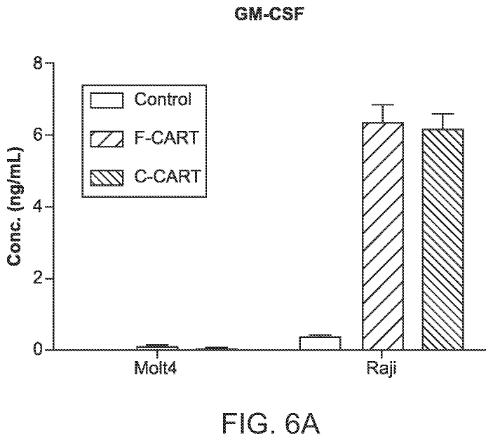

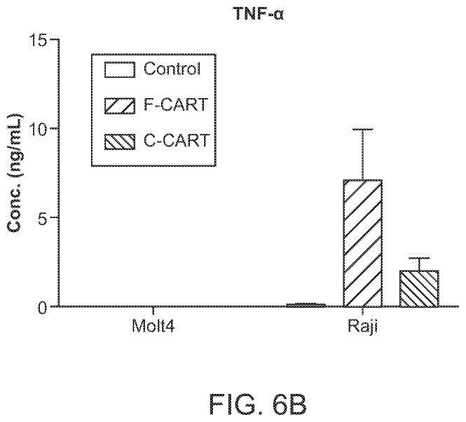

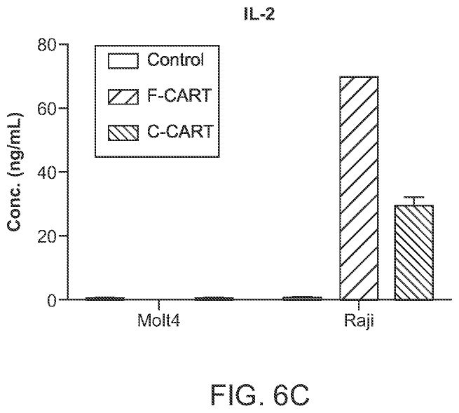

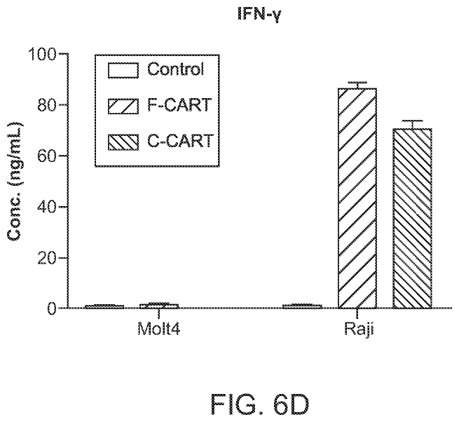

Subsequently, 100 μL of both effector cells (control T cells, F-CART cells and C-CART cells) and the target cells (Molt 4 cells or Raji cells) were added to each well of a 96-well plate at a ratio of 1:1, and incubated together at 37° C., 5% CO₂ for 18-24 hours. After incubation, the cells were subject to centrifuge at 300 g for 5 min. 100-150p of the supernatant was collected and transferred to a new 96-well plate to detect release of the cytokines including GM-CSF, IL-2, TNF-α, and IFN-γ by ELISA, as shown in FIG. 6.

From FIG. 6, it can be seen that there is no significant difference in the level of GM-CSF released between F-CART and C-CART. IL-2 is a factor released by activated T cells, and the level of IL-2 released by F-CART was significantly higher than that of C-CART, indicating that F-CART has a potent activation by tumor antigen stimulation. The levels of TNF-α and IFN-γ both indicate the direct killing efficacy of T cells for target cells, and the levels of both these two factors were significantly higher in F-CART than those in C-CART, indicating superior killing activity of F-CART than C-CART.

### Example 10: In Vivo Tumor Killing Efficacy

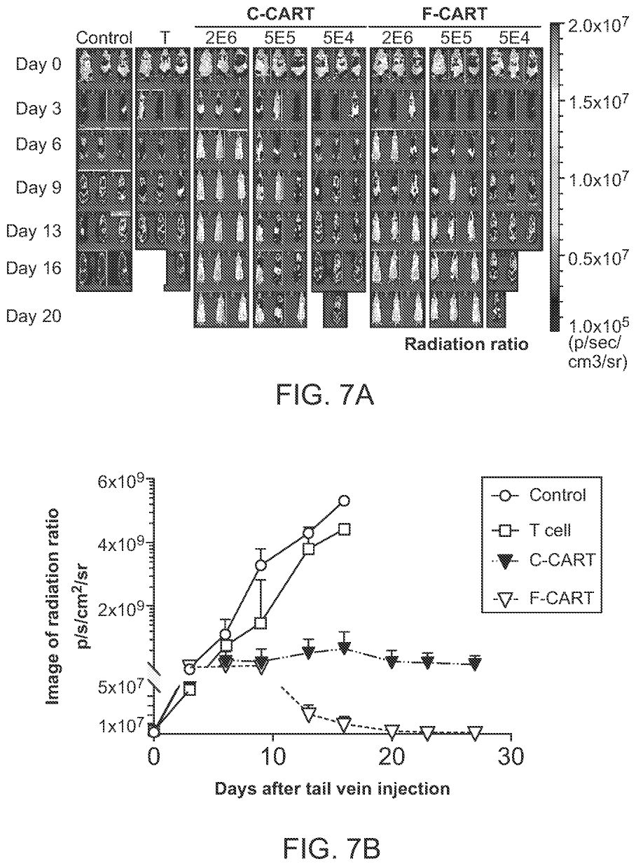

A CDX model (allograft tumor model) of immuno-deficient mice (NDG mice) was established by using CD19⁺ Raji B malignant cell line. Briefly, Raji-Luc cells were suspended in PBS to a density of 5×10⁵ cells/0.2 mL, and 0.2 mL of the cells were injected to each B-NDG (B-NSG) mice through tail vein. Since day 0 of the injection, the mice were subject to imaging to observe growth of the tumor. When the average signal of the image of the mice reached to about 5×10⁶ P/S, the mice with moderate signals was selected and then randomly divided into different groups, with 3 mice per group. On the same day, the mice were subject to tail vein injection by using F-CART cells (such as F-CART-FV2 cells), C-CART cells, the un-transfected T cells (starting material) and blank with cell cryopreservation solution only. After injection, growth of tumors in each animal was observed twice a week by imaging, and the results are as shown in FIGS. 7A and 7B. From the result it can be seen that, F-CART showed a significantly stronger inhibition on the tumors of the mice, and eventually eliminated the tumor. As shown in FIG. 7B, by using a dose of 5×10⁵(5E5), C-CART showed certain tumor inhibitory effect, but was not able to eliminate the tumor, meanwhile, F-CART completely eliminated the tumor and provided a stronger anti-tumor efficacy.

### Example 11: In Vivo Toxicity

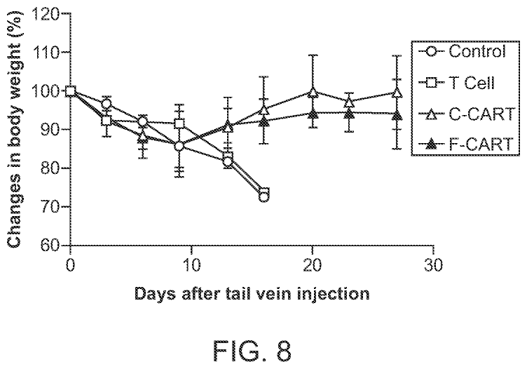

Body weight of the mice were measured before and after administrating the F-CART cells (such as F-CART-FV2 cells), C-CART cells, un-transfected T cells (starting material) and blank as Example 10 at a dose of 2×10⁶(2E6). Changes in body weight of the animals before and after the administration were compared, as shown in FIG. 8. It can be seen from FIG. 8 that changes in body weight (%) represents the body weight after administration as a percentage of the original body weight, calculated as changes in body weight (%)=body weight after administration/original body weight×100%.

The result shows that compared to the blank and the starting material groups, body weight of the animals in F-CART and C-CART groups did not show significant change, and the body weight of the animals in F-CART group appeared to be more stable, indicating the non-obvious toxicity of the preparation.

### Example 12: In Vivo Dose-Dependent Tumor Suppression

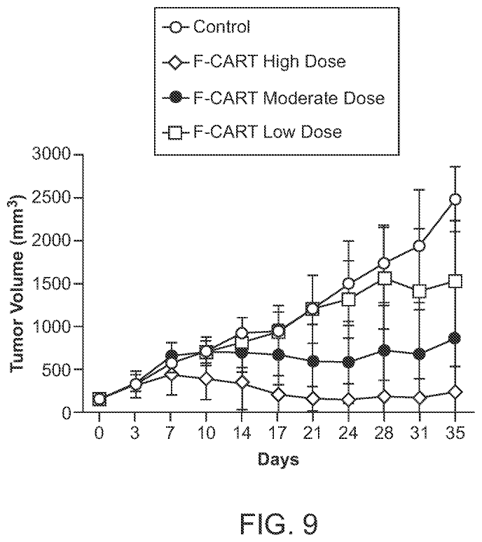

F-CART cells (such as F-CART-FV2 cells) at high (2×10⁶ cells/0.2 mL), moderate (5×10⁵ cells/0.2 mL) and low (5×10⁴ cells/0.2 mL) doses and blank were administrated to the mice as established in Example 10 to observe changes in the size of the tumors. The result is as shown in FIG. 9. It can be seen in FIG. 9 that the inhibition of F-CART cells on tumors was dose-dependent, and the size of the tumors decreased as the dose administrated increased.

### Example 13: In Vivo Proliferation of CAR-T Cells

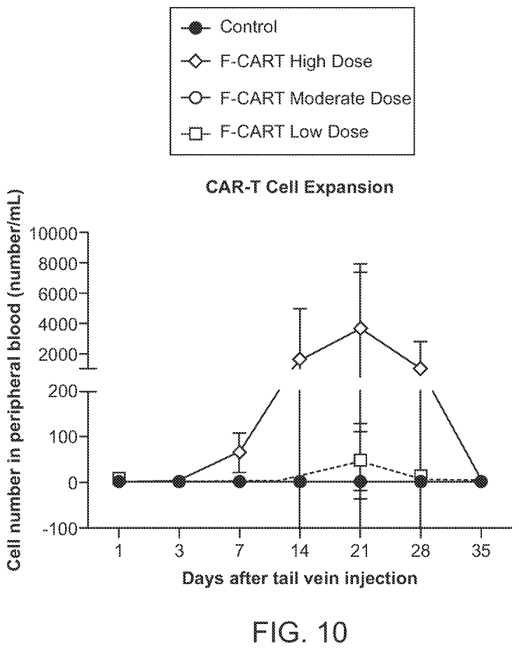

F-CART cells (such as F-CART-FV2 cells) at high (2×10⁶ cells/0.2 mL), moderate (5×10⁵ cells/0.2 mL) and low (5×10⁴ cells/0.2 mL) doses and blank were administrated to the mice as established in Example 10, and then peripheral blood of the mice was collected to analyze the expansion of the CAR-T cells by flow cytometry (see Garcia R L et al., Analysis of proliferative grade using anti-PCNA/cyclin monoclonal antibodies in fixed, embedded tissues. Comparison with flow cytometric analysis. The American journal of pathology, 1989, 134(4): 733). The result is as shown in FIG. 10.

It can be seen from FIG. 10 that F-CART cells showed rapid expansion until 28 days after the administration, especially between 14 and 21 days. The expansion capability of the F-CART cells was dose-dependent, and increased with the dose administrated.

### Example 14: Subpopulations of F-CART Cells Versus C-CART Cells

T cell status as well we the exhaustion were evaluated to compare the subpopulations of the F-CART (such as F-CART-FV2) and C-CART preparations. Subpopulations of the F-CART cells (such as F-CART-FV2 cells) and C-CART cells were analyzed by flow cytometry (see Garcia R L et al., Analysis of proliferative grade using anti-PCNA/cyclin monoclonal antibodies in fixed, embedded tissues. Comparison with flow cytometric analysis. The American journal of pathology, 1989, 134(4): 733).

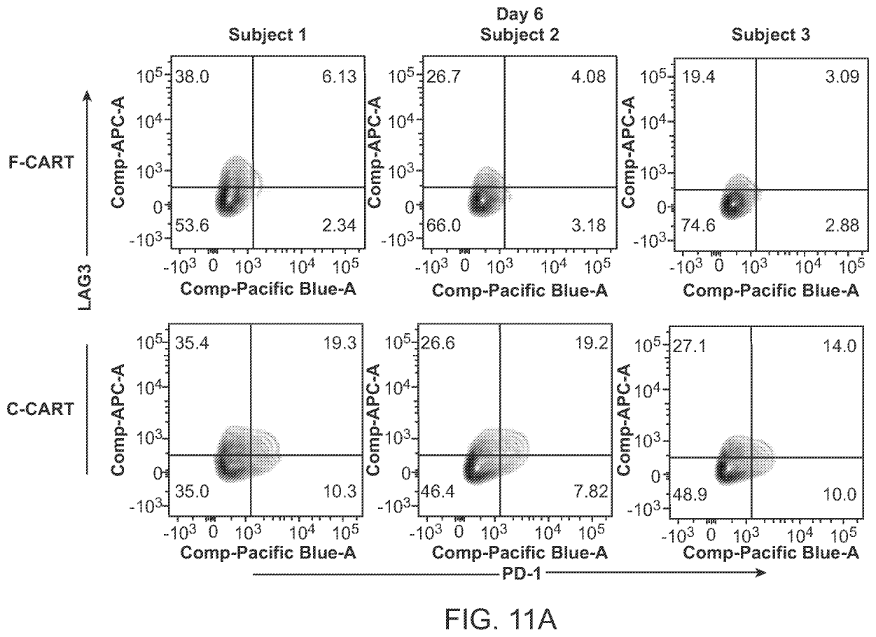

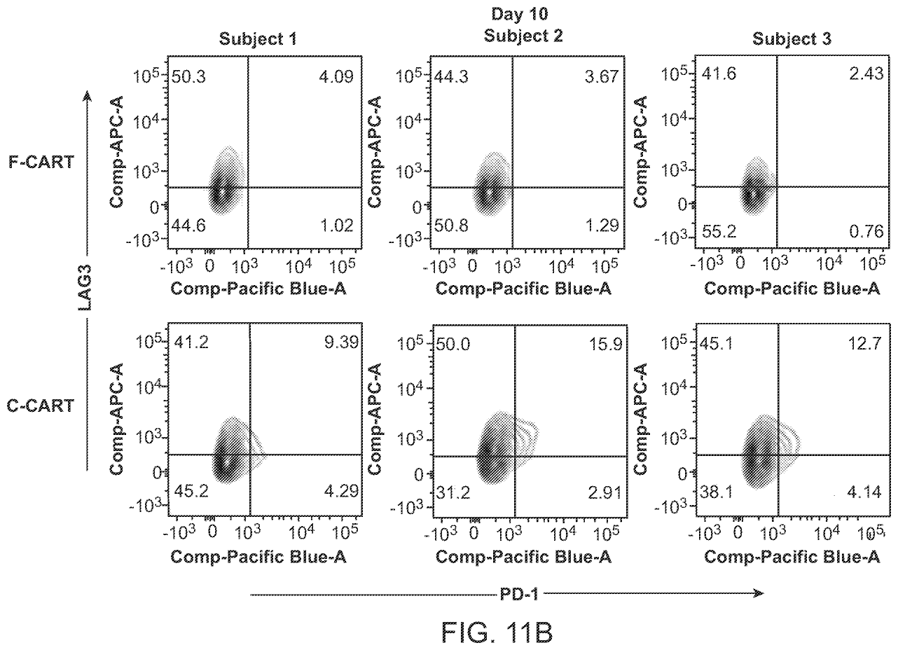

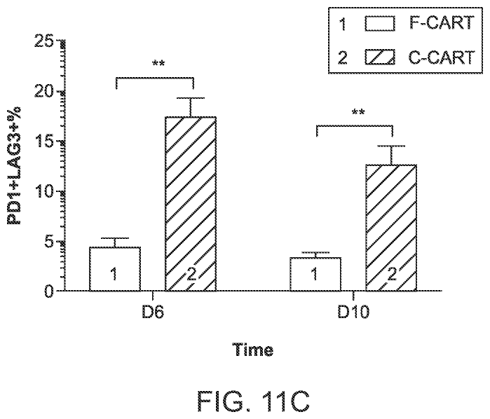

Briefly, cells were collected from three healthy donors and F-CART cells and C-CART cells were prepared as described in Example 2 and 4. Then 1×10⁶ prepared cells from each group were co-cultured with radiated K562-CD19 cells at a 1:1 ratio for 10 days, respectively. During the culture, the radiated K562-CD19 cells were supplemented every 3 days so that to keep the 1:1 ratio. On day 6 and day 10, the expression of PD1 and LAG3 on the surface of F-CART and C-CART cells was analyzed by flow cytometry. The result is as shown in FIG. 11A and FIG. 11B. From FIG. 11A and FIG. 11B, it can be seen that the proportion of PD1⁺LAG3⁺ cells in F-CART group was significantly lower than that of C-CART on both day 6 and day 10, indicating fewer cells were inhibited or exhausted in the F-CART group.

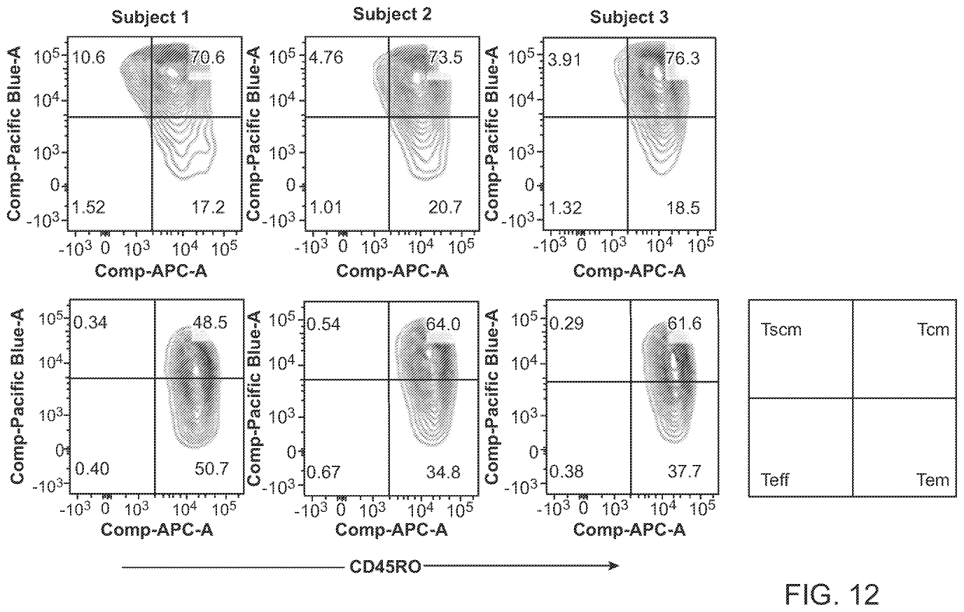

Similarly, cells were collected from three healthy donors and F-CART cells and C-CART cells were prepared as described in Example 2 and 4. Then 1×10⁶ prepared cells from each group were co-cultured with radiated K562-CD19 cells with a 1:1 ratio for 10 days, respectively. During the culture, the radiated K562-CD19 cells were supplemented every 3 days so that to keep the 1:1 ratio. Then the expression of CD62L and CD45RO on the surface of F-CART and C-CART cells was analyzed by flow cytometry to evaluate the differentiation status of the cells. The results are as shown in Table 6 and FIG. 12.

TABLE 6
Subpopulations of F-CART versus C-CART

| | F-CART ( Mean ± SD) | C-CART ( Mean ± SD) |
|---|---|---|
| TSCM | 6.42 ± 3.64* | 0.39 ± 0.13 |
| TCM | 73.47 ± 2.85* | 58.03 ± 8.34 |
| TEM | 18.8 ± 1.77** | 41.06 ± 8.47 |
| TEFF | 1.28 ± 0.26* | 0.48 ± 0.16 |

*P <0.05,
**P <0.01

From the results, it can be seen that the proportions of TSCM and TCM cells in the F-CART group was higher than those of C-CART group, and the proportion of TEM cells is lower, indicating less extent of differentiation, younger status, as well as stronger proliferation and differentiation potentials of the F-CART cells.

### Example 15: Safety and Clinical Efficacy of the F-CART Cell Preparation

The F-CART cells were administered to a human patient XF001 to study the safety and efficacy of the preparation. Patient XF001, female, 39 years old, height 150 cm, weight 70 kg, diagnosed with chronic myeloid leukemia (CML) for 15 years. B cell acute lymphoblastic leukemia (B-ALL) together with central nervous system leukemia was found and diagnosed in the patient 4 months before the F-CART treatment. The patient also had a drug resistance mutation T315I/V299L in BCR-ABL. Effects of various chemotherapy treatments were poor, and the patient also developed resistance to tyrosine kinase inhibitors and failed to respond to conventional chemotherapy.

Leukocytes were collected from the patient, and F-CART cells derived from the patient comprising CD19-CAR were prepared according to the method of Example 2. The interval from apheresis to cell infusion was only 10 days, and the preparation process of the F-CART cells was only 24 hours. The patient was subjected to a 3-day FC chemotherapy pretreatment (on day 1-3, daily administration of Fludarabine 50 mg and Cyclophosphamide 300 mg) first, and then the F-CART preparation was infused to the patient at a cell number of 4.2×10⁶ (about 6×10⁴ cells/kg body weight).

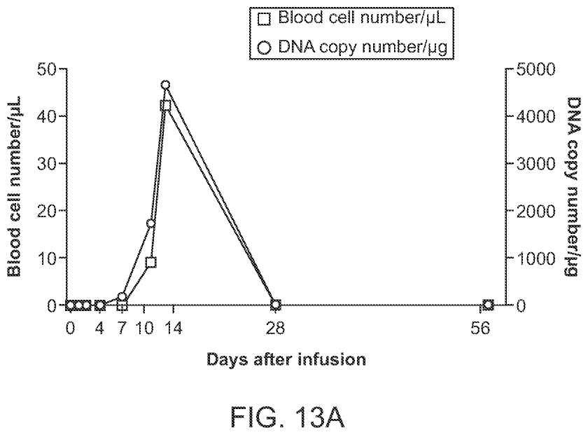

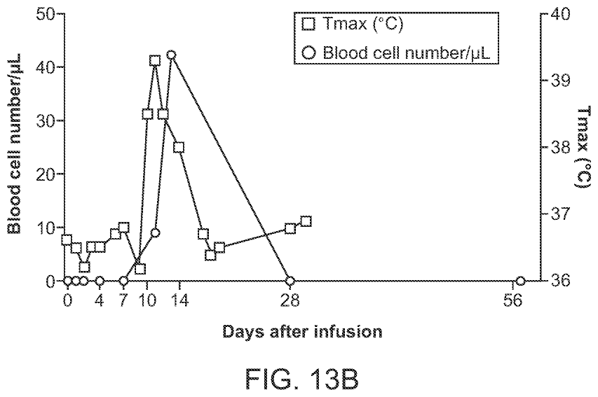

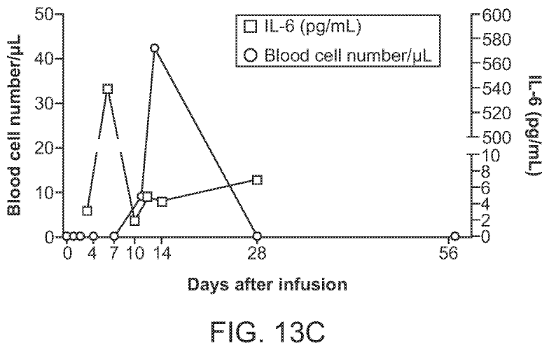

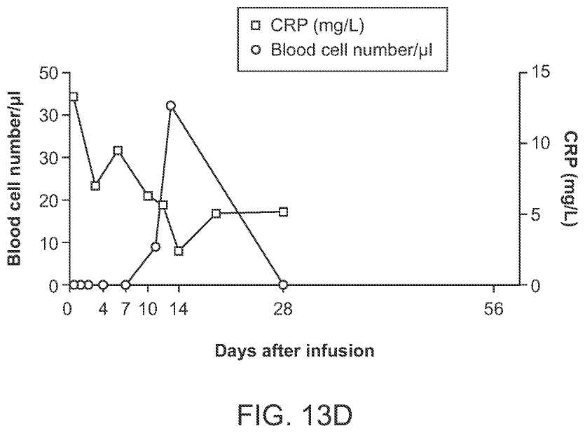

As shown in FIG. 13B, on day 8, the body temperature of the patient was normal, and from day 10 to day 13 after the infusion of the F-CART cells, symptoms such as fever and infection occurred, and the symptoms were judged as first grade cytokine release syndrome (first grade CRS). The patient was then treated with antipyretic therapy, and Meropenem for infection. On day 16, the body temperature of the patient went back to normal (as shown in FIG. 13B).

As shown in FIG. 13A, 1-7 days after the infusion of F-CART, flow cytometry results showed that very few CD19⁺ B cells appeared in peripheral blood, and no copy of the nucleic acid molecule encoding CAR (CAR copy number) was detected by PCR. 8-28 days after the infusion of F-CART, no CD19⁺ B cell was detected in peripheral blood. On day 13 after the infusion of F-CART, the CAR copy number came to the peak (4670.2 copies/pg DNA), and on day 28, the copy number went back to 15.4 copies/pg DNA. On day 56, the copy number decreased to 0. The result of CAR-T analyzed by flow cytometry (changes in the number CAR+ cells) was consistent with the result of CAR copy number.

On day 18 after the infusion of F-CART, analysis of the sample obtained from bone puncture showed that protozoa lymphocytes accounted for 0.5% with normal morphology; granulocyte proliferation was active; minimal residual lesions (MRD) was assessed as CML, indicating clearance of B-ALL MRD in the patient. The patient was thus diagnosed as chronic phase CML (CP-CML). The patient received HLA7/12 semi-compatible hematopoietic stem cell transplantation (HSCT) from her son 40 days after the infusion of F-CART, and showed white blood cell reconstruction and left the hospital 20 days after the transplantation. Changes in the factors of the patient associated with immune response (such as IL-6 and C reaction protein CRP) were as shown in FIG. 13C and FIG. 13D.

From the above results, it was found that after administrating a low dose of the F-CART cells to a patient, the MRD of the B-ALL was successfully eliminated without causing severe CRS and neurotoxicity.

Based on the rapid preparation, the infusion of the F-CART of the present application was accelerated by at least 7-10 days compared to the conventional CD19 CAR-T (C-CART), suggesting advantages of the F-CART in the timing of the treatment. In addition, compared to the conventional CD19 CAR-T (C-CART), the peaks of CAR+ ratio and CAR copy number appeared at a later time point in the patient by using the F-CART preparation.

### Example 16: Safety and Clinical Efficacy of the F-CART Cell Preparation

The F-CART cells were administered to a human patient F01 to study the safety and efficacy of the preparation. After enrollment, PBMCs were isolated from the patient, and F-CART cells derived from the patient comprising CD19-CAR were prepared according to the method described in Example 2. The patient was pretreated with chemotherapy for 3 days (Fludarabine 50 mg×3 days+Cyclophosphamide 0.4 g×3 days+Cytarabine 0.5 g×4 days) first, and then the prepared F-CART cells were infused to the patient at a dose of about 1.07×10⁵ cells/kg body weight.

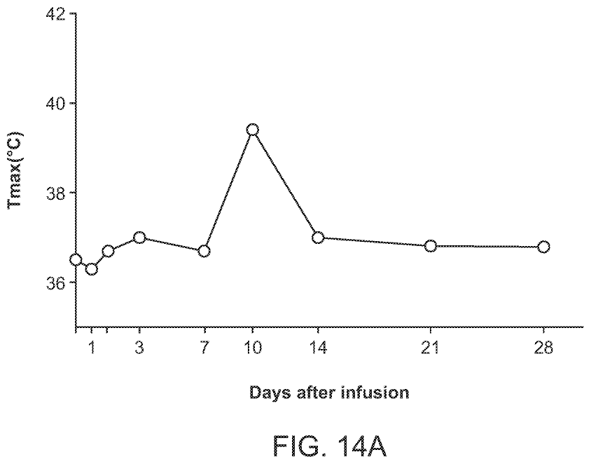

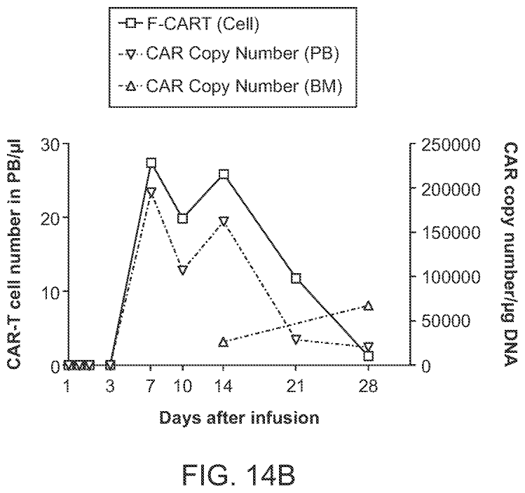

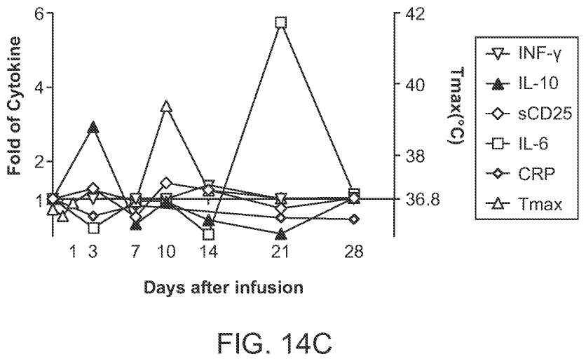

As shown in FIG. 14A, on day 10 after the infusion of F-CART, the patient developed fever, and the body temperature was up to 39.4° C. The fever was relieved after treatment. No hypoxic or hypotensive symptoms, CRS manifestation, or neurotoxicity were found. The patient was not subject to tocilizumab or any other hormonal drugs. The CRS was judged as the first grade. In addition, changes in cytokines including CRP, IL-6, IL-10, INF-γ, and sCD25 in peripheral blood of patients were assayed, as shown in FIG. 14C. Compared to the baseline, the secretion of IL-6 increased on day 21 after the infusion of F-CART. Other cytokines did not show significant changes.

As shown in FIG. 14B, a significant proliferation of F-CART cells was observed in peripheral blood (PB) from day 7 to day 14 after the infusion. On day 7 after the infusion, CAR copy number (qPCR) and F-CART cell number (FACS) in peripheral blood were 195,297 copies/pg DNA and 27.5 cells/μl, respectively; on day 10 after the infusion, CAR copy number (qPCR) and F-CART cell number (FACS) in peripheral blood were 106822 copies/pg DNA and 20 cells/μl, respectively; on day 14 after the infusion, CAR copy number (qPCR) and F-CART cell number (FACS) in peripheral blood were 162464 copies/pg DNA and 26.5 cells/μl, respectively. The proliferation was significantly decreased on day 21, and the F-CART cells could still be detected on day 28 after the infusion. On day 14 and 28 after the infusion, bone marrow (BM) CAR copy numbers were detected as 26429 copies/pg DNA and 68135.6 copies/pg DNA, respectively, and no CAR expansion was detected by qPCR. In addition, on day 14 and 28 after the infusion of the F-CART cells, abnormal B cells in the peripheral blood of the patient could not be detected, and no abnormal cells or tumors appeared in bone marrow sample by flow cytometry.

It can be seen that administrating a low dose of the F-CART cells to the patient is safe, and is effective in killing tumor cells in vivo.

### Example 17: Clinical Efficacy of the F-CART Cell Preparation

The F-CART cell preparation was administered to human patients DF06, GF001, XF002, TF003, TF002, DF04, DF01, XF001, and TF001, which were diagnosed as relapsed or refractory B-ALL patients, respectively.

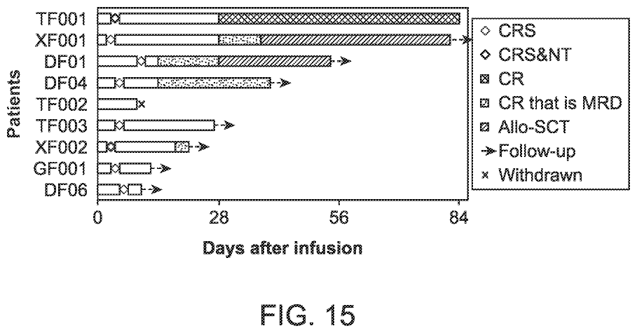

Leukocytes were collected from the patients, and F-CART cells derived from each patient comprising CD19-CAR were prepared according to the method described in Example 2. The interval from apheresis to cell infusion was only 10 days, and the preparation process of the F-CART cells was only 24 hours. During the treatment, the prepared F-CART cells were infused to each of the patients at a dose of about 10⁴ to 10⁷ (about 10′ cells/kg body weight to about 10⁶ cells/kg body weight). The results are as shown in FIG. 15.

In the treatment, patient TF002 withdrew from the study on day 10 after the infusion of F-CART. Five patients (TF001, XF001, DF01, DF04, and XF002) achieved complete remission (CR), and 4 of them (XF001, DF01, DF04, and XF002) achieve minimal residual disease (MRD). These results demonstrate the effectiveness of the F-CART cell preparation in tumor treatment.

In addition, 8 of the 9 patients developed CRS (cytokine release syndrome) (except for patient TF002), and only 2 patients (TF001 and XF002) developed CRS of grade 3 or higher, and grade 1 neurotoxicity (NT). The CRS in the 8 patients occurred between day 3 and day 10 after the infusion. These results indicate that the F-CART cell preparation is relatively safe.

### Example 18: Phenotypic Analysis of F-CART Vs C-CART

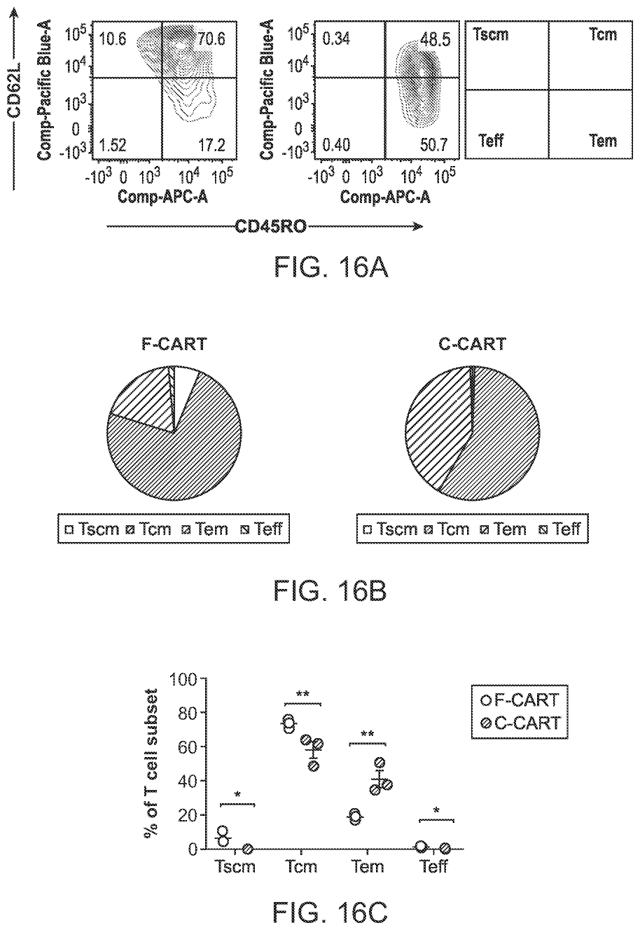

Lymphocyte subpopulations of C-CART and F-CART cells were analyzed by conventional flow cytometry. Expression of markers CD3, CD4, CD8, CD45RO and CD62L were analyzed through flow cytometry by using 2-3×10⁶ of starting C-CART cells and F-CART cells, respectively. The results are as shown in Table 7 and FIG. 16A, FIG. 16B, and FIG. 16C.

TABLE 7
C-CART vs F-CART

| | F-CART (Mean ± SD) | C-CART (Mean ± SD) |
|---|---|---|
| TSCM | 6.42 ± 3.64 * | 0.39 ± 0.13 |
| TCM | 73.47 ± 2.85 * | 58.03 ± 8.34 |
| TEM | 18.8 ± 1.77 ** | 41.06 ± 8.47 |
| TEFF | 1.28 ± 0.26 * | 0.48 ± 0.16 |

In Table 7, TEM represents effector T cells having CD45RO⁺/CD62L⁻; TCM represents central memory T cells having CD45RO⁺/CD62L⁺; T_{N} represents initial (or naive) T cells having CD45RO⁻/CD62L⁺ with great differentiation potential, which are able to differentiate into cell subpopulations such as TEM and TCM.

The result shows that the proportion of TSCM and TCM are more abundant in FasT CAR-T population. More CD45RO+/CD62L+(TCM) than CD45RO+/CD62L− (TEM) are observed in FasT CAR-T cells (4-fold increase). Additionally, more CD45RO−/CD62L+ (TSCM) in F-CART than in C-CART (31-fold increase) are observed. These results indicate that the F-CART preparation process of the present application can be used to prepare activated T cells with differentiation potential and a “young” phenotype with a non-exhausted phenotype.

### Example 19: In Vitro Expansion, Phenotype, and Cytotoxity of F-CAR-T Vs C-CAR-T Cells

Fold expansion was quantified on day 8, 12, and 18 in F-CART cells and C-CART cells to compare the in vitro proliferation of the two methods.

Subpopulations of C-CART and F-CART cells were also analyzed by conventional flow cytometry. Expression of markers CD3, CD4, CD8, PD-1 and LAG3 were analyzed through flow cytometry by using 2-3×10⁶ of starting C-CART cells and F-CART cells, respectively.

Cytotoxicity of F-CART and C-CART was compared using the real time cell analyzer (RTCA) assay. RTCA is a technique that uses real time cell monitoring to detect migration, cytotoxicity, and adherence/proliferation of cells during direct and indirect co-cultures. Briefly, cocultures of C-CART cultured with CD19⁺ tumor cells, F-CART cultured with CD19⁺ tumor cells, non-transduced cells cultured with CD19⁺ tumor cells, and tumor only cells (Hela-CD19) were set up. Background measurements were taken from the wells by adding 50 μl of the same medium to the E-16 plates. Subsequently, RTCA Software Package 1.2 was used to calibrate the plates. Cells were plated at a density of 20,000/well in fresh medium to a final volume of 200 μl. Cells were incubated for 4 min at 37° C. and 5% CO₂ in the RTCA cradle before the software schedule was initiated. The impedance signals were recorded every 5 min for the first 25 scans (2 h) and every 10 min until the end of the experiment (40 hours). After 20 h of impedance reading, 140 μl of medium was removed from each well and replaced with the appropriate volume of conditioned medium (CM).

Cytokine secretion of IL-2 and IFNγ was evaluated using media from the RTCA assay. Briefly, 100 uL of media was collected from the co-culture assay and evaluated by ELISA.

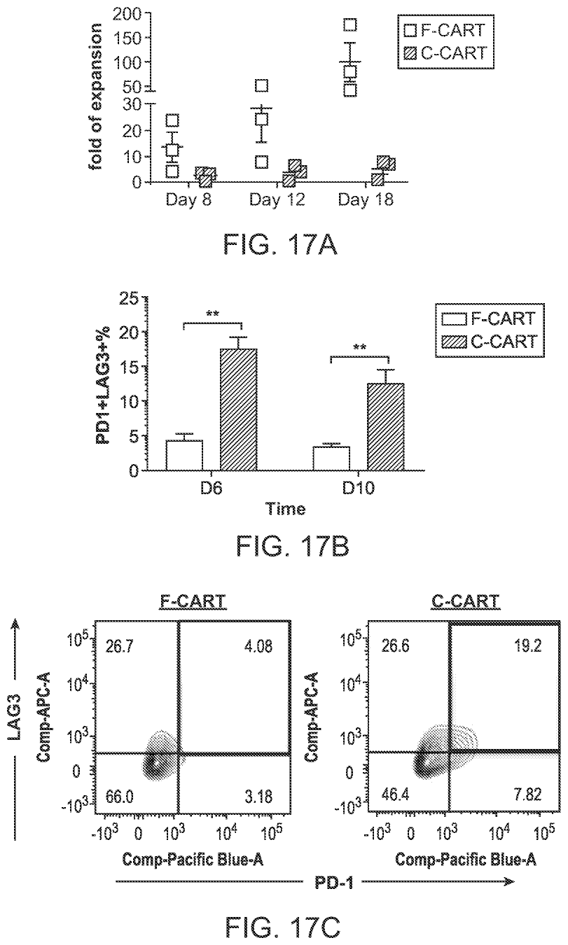

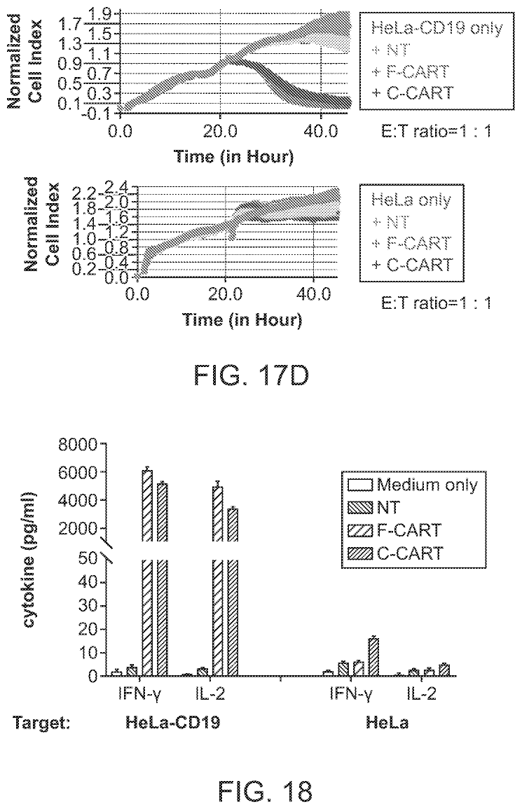

The results are as shown in FIG. 17A, FIG. 17B, FIG. 17C, FIG. 17D, and FIG. 18. The results show that the % of PD1+LAG3+ CAR-T cells are significantly less compared to conventionally-manufactured CAR-T. Results also show that F-CART exhibit CD19 specific killing, tumor-specific cytokine secretion, and similar killing capacity compared to C-CART.

### Example 21: In Vitro Analysis of F-CART Vs C-CART Subject Samples

The CART cells were prepared using the F-CART method and conventional method for subjects in Table 8.

TABLE 8
Summary of Patient Samples

| ID | Cancer |
|---|---|
| GC007-180016 | B-ALL |
| GC007-180018 | B-ALL |
| GC007-180019 | B-ALL |
| GC007F-190007 | B-ALL |
| GC007-190004 | B-ALL |
| GC022-190003 | B-ALL |

### Cellular Expansion

Leukocytes were collected from the patients, and F-CART and C-CART cells from each subject expressing a CD19-CAR were prepared according to the method described in Example 2. The interval from apheresis to cell infusion was only 10 days, and the preparation process of the F-CART cells was only 24 hours.

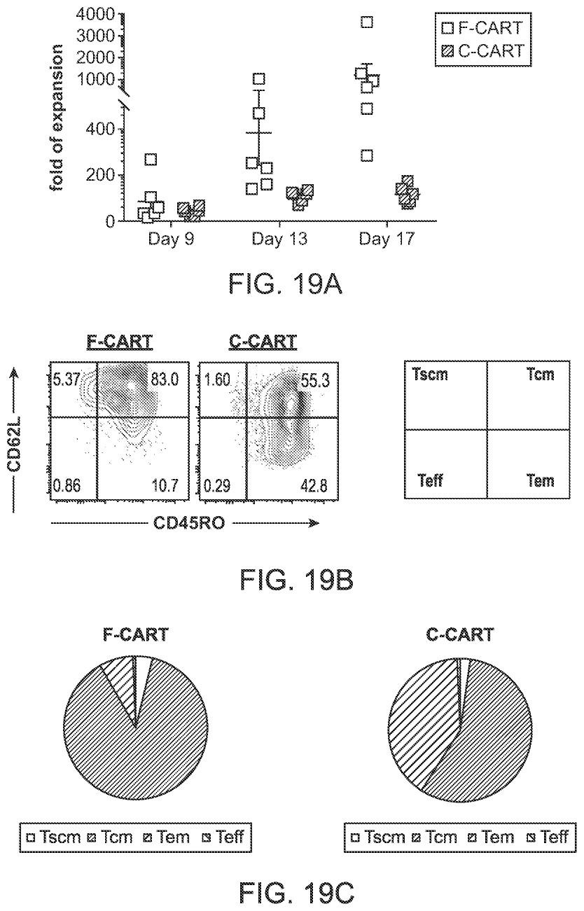

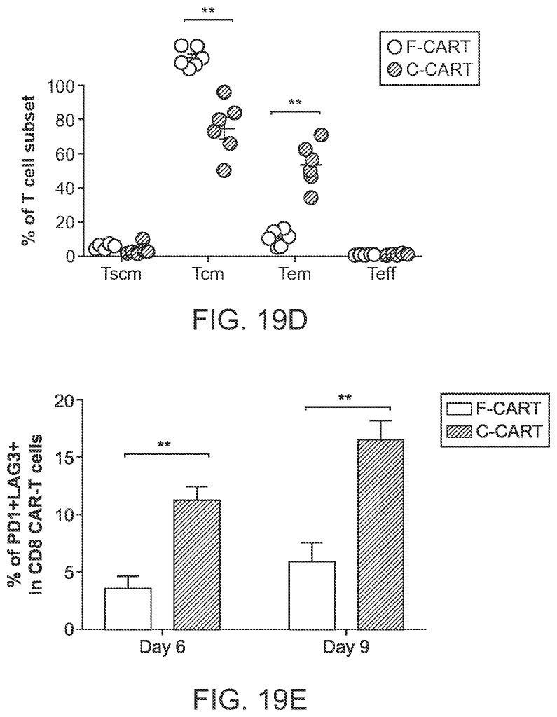

Expansion of the F-CART and C-CART was compared under the stimulation of tumor antigen. Briefly, primary tumor cells expressing CD19 (from B-ALL tumor patient) were co-cultured with F-CART cells and C-CART cells since day 0. Cell viability and living cell numbers were calculated on days 9, 13, and 17. Cell density was maintained between 0.5×10⁶ cells/mL and 1.0×10⁶ cells/mL during the process. The result is as shown in FIG. 19A. Results show that FasT CAR-T (F-CART) proliferates drastically better compared to conventionally manufactured CAR-T (C-CART).

### Phenotype

Lymphocyte subpopulations of C-CART and F-CART cells from subject GC007F were analyzed by conventional flow cytometry. Expression of markers CD3, CD4, CD8, CD45RO and CD62L were analyzed through flow cytometry by using 2-3×10⁶ of starting C-CART cells and F-CART cells, respectively. The results are as shown in FIG. 19B, FIG. 19C, FIG. 19D, and Table 9. Results show that TCM are more abundant in FasT CAR-T population as compared to C-CART.

TABLE 9
Cellular Phenotype of GC007F

| | F-CART (Mean ± SD) | C-CART (Mean ± SD) |
|---|---|---|
| TSCM | 3.84 ± 1.11 | 2.34 ± 2.36 |
| TCM | 87.92 ± 3.98 ** | 56.62 ± 10.93 |
| TEM | 7.84 ± 3.18 ** | 40.48 ± 8.86 |
| TEFF | 0.42 ± 0.24 | 0.59 ± 0.34 |

Additionally cellular exhaustion was also determined using flow cytometry. Lymphocyte subpopulations of C-CART and F-CART cells from subject GC007F were analyzed by conventional flow cytometry. Expression of markers CD3, CD8, PD-1, and LAG3 were analyzed through flow cytometry in CAR positive cells. The results are shown in FIG. 19E. Results show that % of PD1+LAG3+ CAR-T cells are significantly less compared to conventionally-manufactured CAR-T.

### Cytotoxicity RTCA and ELISA

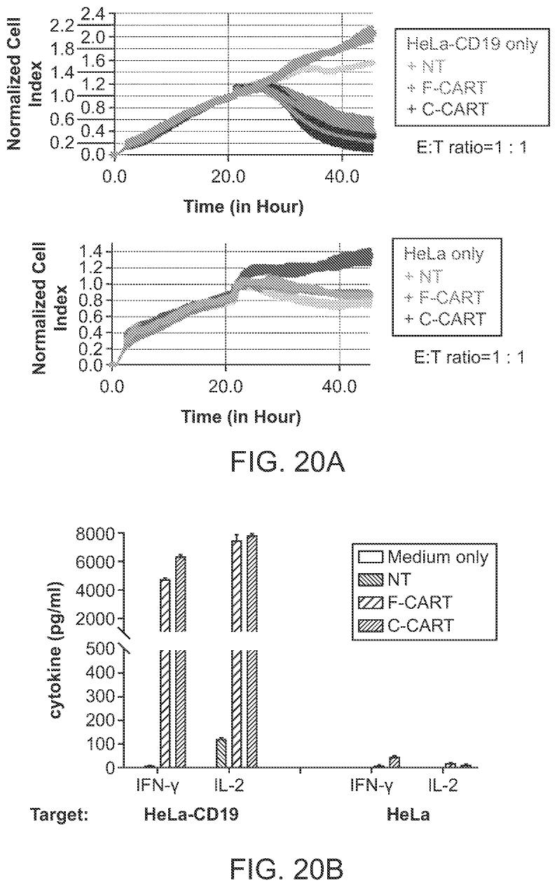

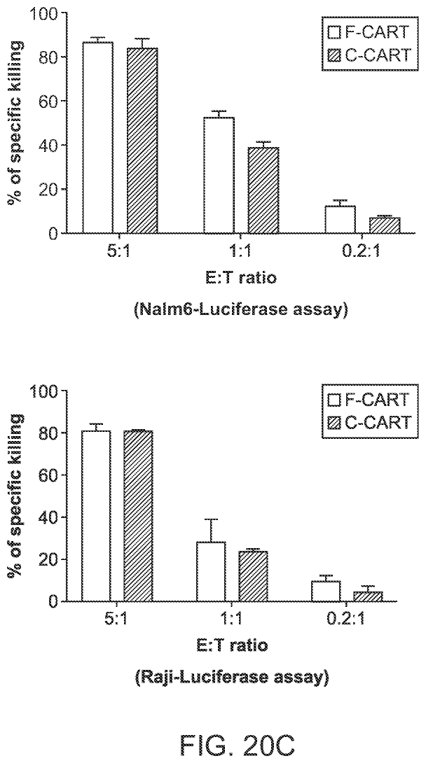

Cytotoxicity of subject's GC007F F-CART and C-CART was compared using the real time cell analyzer (RTCA) assay as previously described in Example 17 using an effector to target ratio of 1:1. Results are shown in FIG. 20A.

Cytokine secretion of IL-2 and IFNγ was evaluated using media from the RTCA assay. Briefly, 100 uL of media was collected from the co-culture assay and evaluated by ELISA. The results are as shown in FIG. 20B. Results also show that F-CART exhibit CD19 specific killing, tumor-specific cytokine secretion, and similar killing capacity compared to C-CART.

### Cytotoxicity Luciferase Assay

Luciferase-expressing NALM-6 or Raji tumor cells were placed in 96-well round bottom plates at a concentration of 3×10⁵ cells/ml in triplicates, were given D-firefly luciferin potassium salt (75 pg/ml; Caliper Hopkinton, MA), and measured with a luminometer. This was done to establish the BLI baseline readings before the occurrence of any cell death and to ensure equal distribution of target cells among wells. Subsequently, effector F-CART or C-CART cells were added at 5:1, 1:1, and 0.2:1 effector-to-target (E:T) ratios and incubated at 37° C. for 2 or 4 hours. BLI was then measured for 10 seconds with a luminometer (Packard Fusion Universal Microplate Analyzer, Model A153600) as relative light units (RLU). Cells were treated with 1% Nonidet P-40 (NP40) or with water as a measure of maximal killing. Target cells incubated without effector cells were used to measure spontaneous death RLU. Cells were images at 2 hours or 4 hours. Triplicate wells were averaged and percent lysis was calculated from the data with the following equation: % specific lysis=100×(spontaneous death RLU−test RLU)/(spontaneous death RLU−maximal killing RLU).

Results are shown in FIG. 20C and show comparable in vitro cytotoxicity between F-CART and C-CART, therefore the method of manufacture of concurrent transduction and activation does not significantly affect cytotoxicity of engineered cells while reducing manufacturing time. Table 10 below shows a summary of the various in vitro and vivo findings of comparative studies between F-CART and C-CART.

TABLE 10
Summary of F-CART vs. C-CART

| Performance Feature | F-CART | C-CART |
|---|---|---|
| Proliferation | ++++ | + |
| Memory/Stemness | +++ | + |
| Exhaustion | + | +++ |
| Killing Capacity (in vitro) | +++ | +++ |
| Killing Capacity (in vivo) | +++ | + |
| BM Migration | +++ | + |

### Example 22: In Vivo Analysis of F-CART Vs C-CART in Murine Leukemia Model

NOG mice (NOD.Cg-Prkdcscid Il2rgtm1Sug/JicTac) were engrafted with Raji-Luc cells. Briefly, Raji-Luc cells were suspended in PBS to a density of 5×10⁵ cells/0.2 mL, and 0.2 mL of the cells were injected to each mouse through tail vein. Since day 0 of the injection, the mice were subject to imaging to observe growth of the tumor. When the average signal of the image of the mice reached to about 5×10⁶ P/S, the mice with moderate signals was selected and then randomly divided into different groups, with 3 mice per group. On the same day, the mice were subject to tail vein injection by using F-CART cells, C-CART cells, the un-transfected T cells, and blank with cell cryopreservation solution only. After injection, growth of tumors in each animal was observed twice a week via bioluminescence imaging.

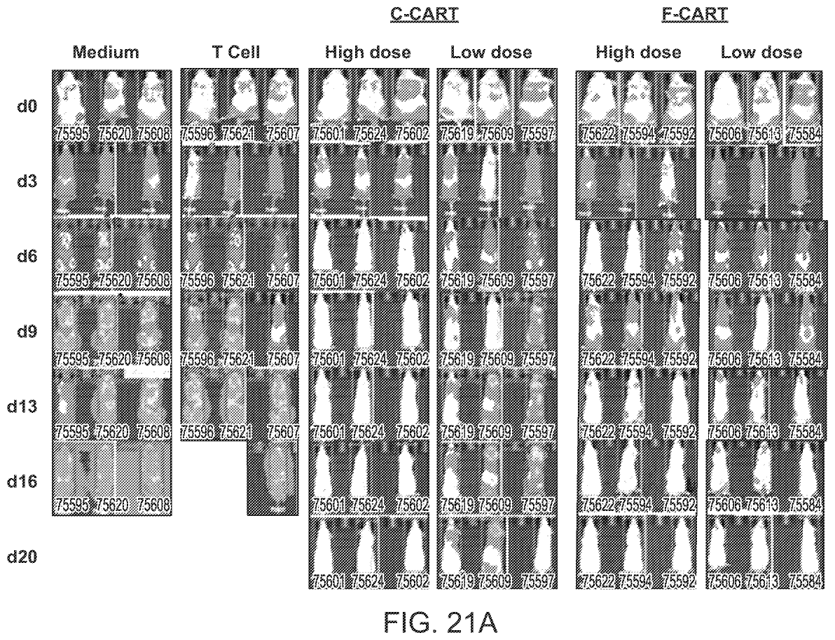

Results are as shown in FIG. 21A. From the result it can be seen that, F-CART showed a significantly stronger reduction of tumors, and eventually eliminated the tumor.

### Example 23: In Vivo Analysis of Infiltration and Chemotaxis of F-CART Vs C-CART in Murine Leukemia Model

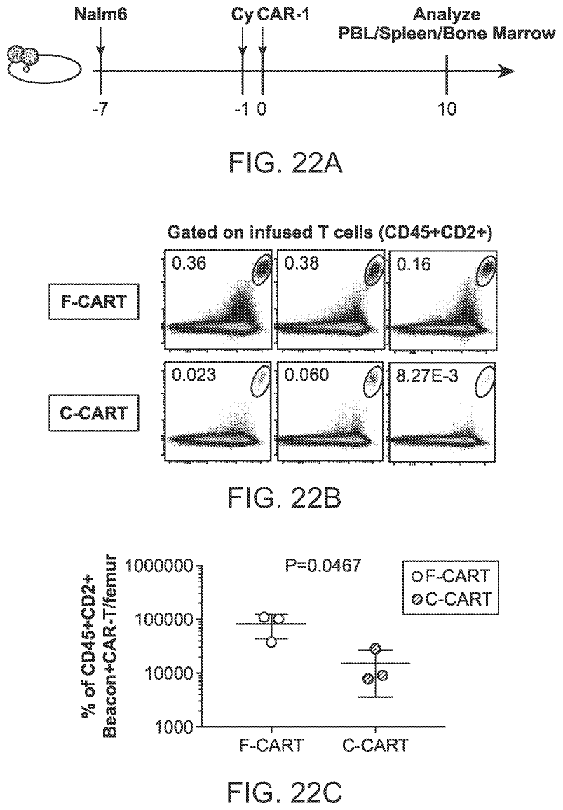

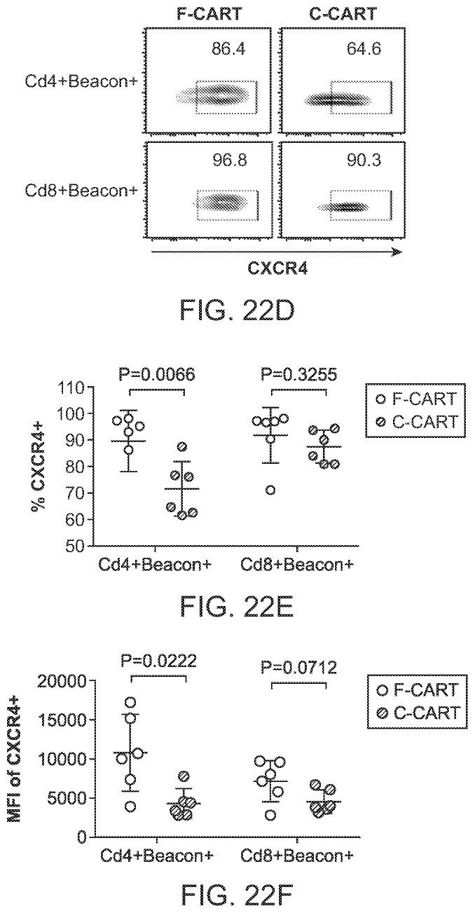

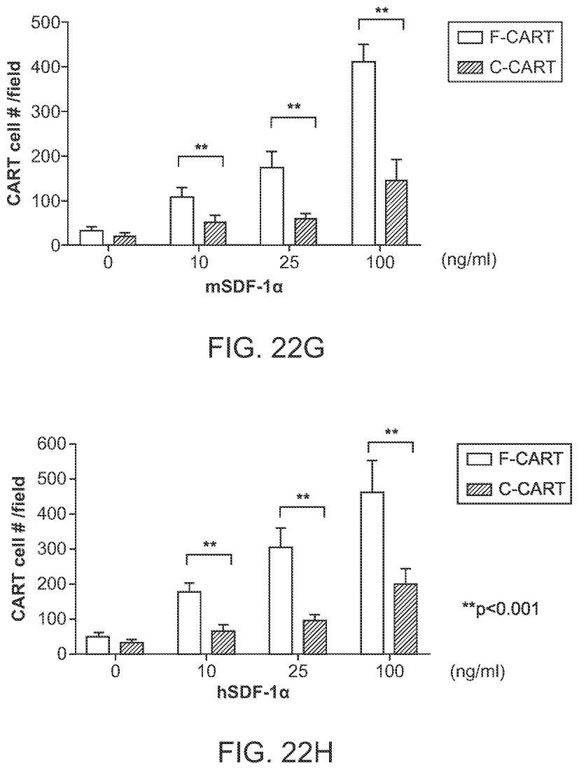

NOG mice (NOD.Cg-Prkdcscid Il2rgtm1Sug/JicTac) were engrafted with NALM-6-Luc cells. Briefly, NALM-6 cells were suspended in PBS to a density of 5×10⁵ cells/0.2 mL, and 0.2 mL of the cells were injected to each mouse through tail vein injection. 7 days post tumor cell injection, mice were subject to tail vein injection of F-CART cells, C-CART cells, the un-transfected T cells, and blank with cell cryopreservation solution only. After treatment, infiltration of cells into the bone marrow was evaluated on day 10 by isolating bone marrow from the femur of the mice and evaluating the sample for the presence of CAR positive cells. Treatment schematic is depicted in FIG. 22A. Results are shown in FIG. 22B, and FIG. 22C. Results show that there exists dramatically more infiltration of F-CART in the bone marrow as compared to C-CART treated mice 10 days after CAR-T infusion.

Subpopulations of C-CART and F-CART cells were also analyzed using flow cytometry. Expression of markers CD3, CD4, CD8, and CXCR4 were analyzed through flow cytometry. Results are shown in FIG. 22D, FIG. 22E, and FIG. 22F. Results show a higher expression of CXCR4 in F-CART treated mice.

Chemotaxis was investigated using 5 μm pore-size transwell plates (Costar, Cambridge, MA). Five×10⁵ cells were dispensed in the upper chamber, chemokines or medium alone were added to the lower chamber. Mouse SDF-1α and human SDF-1α were tested at concentrations of 0 ng/ml, 10 ng/ml, 25 ng/ml, and 100 ng/ml. Plates were incubated 2 h at 37° C. Migrated cells were collected and counted using CFSE, and migration index was calculated as follows: (n° of migrated cells/n° of dispensed cells)×100. Migration index obtained with medium alone was subtracted from each value. Results are shown in FIG. 22G and FIG. 22H. Results show that more CFSE labeled F-CART transmigrate to the bottom well in the presence of murine SDF-1a or human SDF-1a as compared to C-CART.

### Example 24: Analysis of T Cells Expressing Engineered T Cell Receptors (TCRT Cells)

### Construction of Lentiviral Vector and Preparation of TCRT Cells

Expression of a gene coding for NY-ESO-1, also known as CTAG1, is limited to germ cells, and such expression is minimal in normal somatic tissue. However, the NY-ESO-1 gene is frequently expressed in cancer, thus can be targeted as a cancer-testis (CT) antigen. Expression of the NY-ESO-1 gene can be found in a variety of cancer types including, but are not limited to, synovial sarcoma, colon cancer, lung cancer, breast cancer, multiple myeloma, etc.

MHC-I antigens are integral membrane glycoproteins expressed at varying levels on a surface of somatic cells. Without wishing to be bound by theory, MHC-I molecules can function by binding one or more peptides from degraded polypeptides, such as endogenous proteins, (i.e., processed antigens) and presenting the processed antigens to T cell receptor (TCR) specific for a particular MHC-I antigen/peptide complex. Human leukocyte antigen (HLA) is a class I molecule of the human major histocompatibility complex (MHC). HLA-A*02 is a human leukocyte antigen serotype within the HLA-A serotype group. In some cases, HLA-A*02 can be the most frequent allele. In some cases, HLA-A*02 can present a fragment of the NT-ESO-1 protein to a TCR of a T cell.

Thus, a cell (e.g., a T cell) can be engineered to express engineered TCR comprising a ligand specific for a fragment of the NT-ESO-1 protein, which fragment may be presented by HLA-A*02 of cancer or tumor cells. Nucleotide sequences encoding the engineered NY-ESO-1 TCR (as set forth in SEQ ID NO: 11, and the polypeptide product in SEQ ID NO: 13) comprise TCR alpha (TCRA) and TCR beta (TCRB) linked by a self-cleavage linker p2a. The engineered NT-ESO-1 TCR is designed to bind NY-ESO-1 peptide 157-165 (SLLMWITQC) (as set forth in SEQ ID NO: 15).

SEQ ID NO: 11 (TCRA + p2a + TCRB):
ATGGAGACCCTGCTGGGCCTGCTGATCCTGTGGCTGCAGCTCCAGTGGG
TGTCCAGCAAGCAGGAGGTGACCCAGATCCCTGCCGCCCTGAGCGTGCC
CGAGGGCGAGAACCTGGTGCTGAACTGCAGCTTCACCGACTCCGCCATC
TACAACCTGCAGTGGTTCCGGCAGGACCCCGGCAAGGGCCTGACCAGCC
TGCTGCTGATCCAGAGCAGCCAGCGGGAGCAGACCAGCGGACGGCTGAA
CGCCAGCCTGGACAAGAGCAGCGGCCGGAGCACCCTGTACATCGCCGCC
AGCCAGCCCGGCGACAGCGCCACCTACCTGTGCGCTGTGCGGCCTACCA
GCGGCGGCAGCTACATCCCCACCTTCGGCAGAGGCACCAGCCTGATCGT
GCACCCCTACATCCAGAACCCCGACCCCGCCGTGTACCAGCTGCGGGAC
AGCAAGAGCAGCGACAAGTCTGTGTGCCTGTTCACCGACTTCGACAGCC
AGACCAATGTGAGCCAGAGCAAGGACAGCGACGTGTACATCACCGACAA
GACCGTGCTGGACATGCGGAGCATGGACTTCAAGAGCAACAGCGCCGTG
GCCTGGAGCAACAAGAGCGACTTCGCCTGCGCCAACGCCTTCAACAACA
GCATTATCCCCGAGGACACCTTCTTCCCCAGCCCCGAGAGCAGCTGCGA
CGTGAAACTGGTGGAGAAGAGCTTCGAGACCGACACCAACCTGAACTTC
CAGAACCTGAGCGTGATCGGCTTCAGAATCCTGCTGCTGAAGGTGGCCG
GATTCAACCTGCTGATGACCCTGCGGCTGTGGAGCAGCCTTggaagcgg
agagggcagaggaagtcttctaacatgcggtgacgtggaggagaatccc
ggccctATGAGCATCGGCCTGCTGTGCTGCGCCGCCCTGAGCCTGCTGT
GGGCAGGACCCGTGAACGCCGGAGTGACCCAGACCCCCAAGTTCCAGGT
GCTGAAAACCGGCCAGAGCATGACCCTGCAGTGCGCCCAGGACATGAAC
CACGAGTACATGAGCTGGTATCGGCAGGACCCCGGCATGGGCCTGCGGC
TGATCCACTACTCTGTGGGAGCCGGAATCACCGACCAGGGCGAGGTGGC
CAACGGCTACAATGTGAGCCGGAGCACCACCGAGGACTTCCCCCTGCGG
CTGCTGAGCGCTGCCCCCAGCCAGACCAGCGTGTACTTCTGCGCCAGCA
GCTATGTGGGCAACACCGGCGAGCTGTTCTTCGGCGAGGGCTCCAGGCT
GACCGTGCTGGAGGACCTGAAGAACGTGTTCCCCCCCGAGGTGGCCGTG
TTCGAGCCCAGCGAGGCCGAGATCAGCCACACCCAGAAGGCCACACTGG
TGTGTCTGGCCACCGGCTTCTACCCCGACCACGTGGAGCTGTCCTGGTG
GGTGAACGGCAAGGAGGTGCACAGCGGCGTGTCTACCGACCCCCAGCCC
CTGAAGGAGCAGCCCGCCCTGAACGACAGCCGGTACTGCCTGTCCTCCA
GACTGAGAGTGAGCGCCACCTTCTGGCAGAACCCCCGGAACCACTTCCG
GTGCCAGGTGCAGTTCTACGGCCTGAGCGAGAACGACGAGTGGACCCAG
GACCGGGCCAAGCCCGTGACCCAGATTGTGAGCGCCGAGGCCTGGGGCA
GGGCCGACTGCGGCTTCACCAGCGAGAGCTACCAGCAGGGCGTGCTGAG
CGCCACCATCCTGTACGAGATCCTGCTGGGCAAGGCCACCCTGTACGCC
GTGCTGGTGTCTGCCCTGGTGCTGATGGCTATGGTGAAGCGGAAGGACA
GCCGGGGCTAA.
SEQ ID NO: 13:
METLLGLLILWLQLQWVSSKQEVTQIPAALSVPEGENLVLNCSFTDSAI
YNLQWFRQDPGKGLTSLLLIQSSQREQTSGRLNASLDKSSGRSTLYIAA
SQPGDSATYLCAVRPTSGGSYIPTFGRGTSLIVHPYIQNPDPAVYQLRD
SKSSDKSVCLFTDFDSQTNVSQSKDSDVYITDKTVLDMRSMDFKSNSAV
AWSNKSDFACANAFNNSIIPEDTFFPSPESSCDVKLVEKSFETDTNLNF
QNLSVIGFRILLLKVAGFNLLMTLRLWSSLGSGEGRGSLLTCGDVEENP
GPMSIGLLCCAALSLLWAGPVNAGVTQTPKFQVLKTGQSMTLQCAQDMN
HEYMSWYRQDPGMGLRLIHYSVGAGITDQGEVPNGYNVSRSTTEDFPLR
LLSAAPSQTSVYFCASSYVGNTGELFFGEGSRLTVLEDLKNVFPPEVAV
FEPSEAEISHTQKATLVCLATGFYPDHVELSWWVNGKEVHSGVSTDPQP
LKEQPALNDSRYCLSSRLRVSATFWQNPRNHFRCQVQFYGLSENDEWTQ
DRAKPVTQIVSAEAWGRADCGFTSESYQQGVLSATILYEILLGKATLYA
VLVSALVLMAMVKRKDSRG.

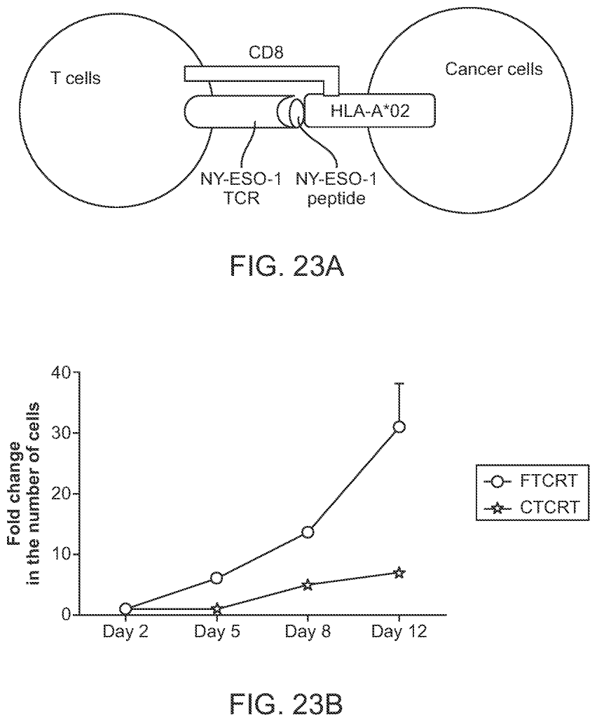

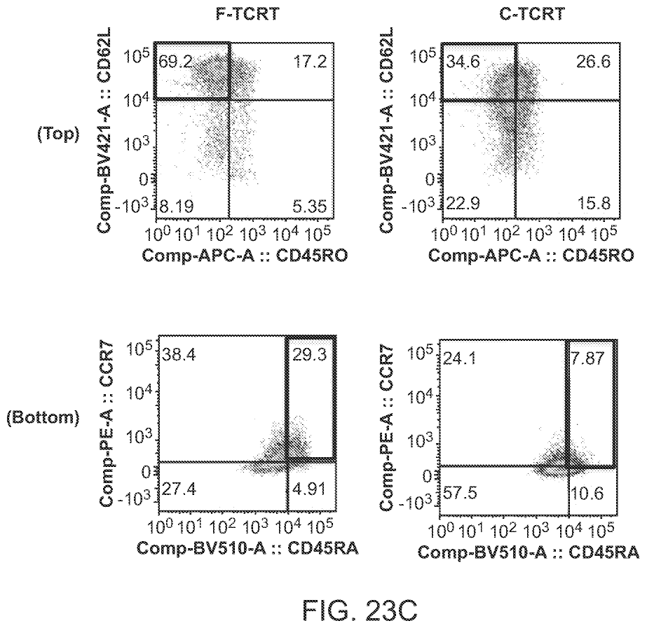

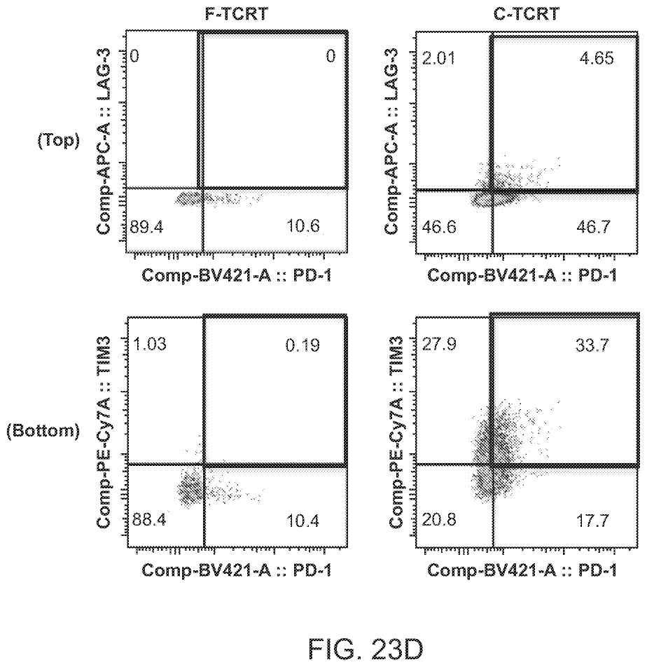

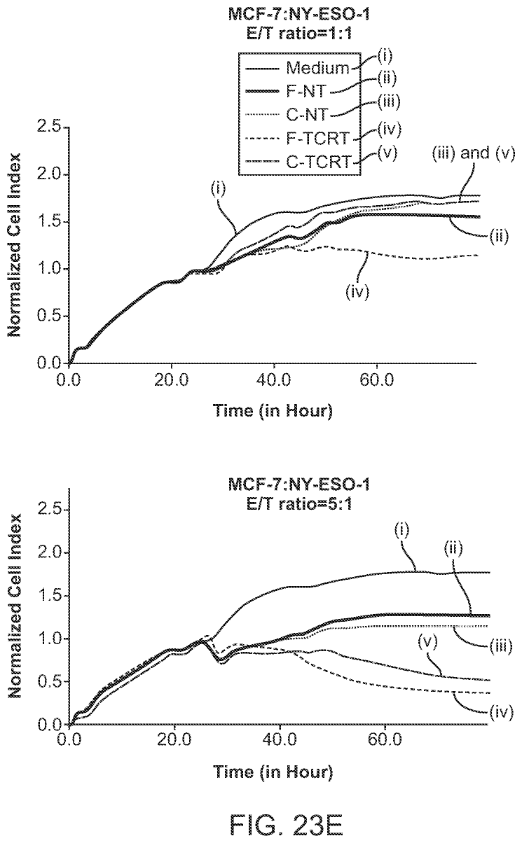

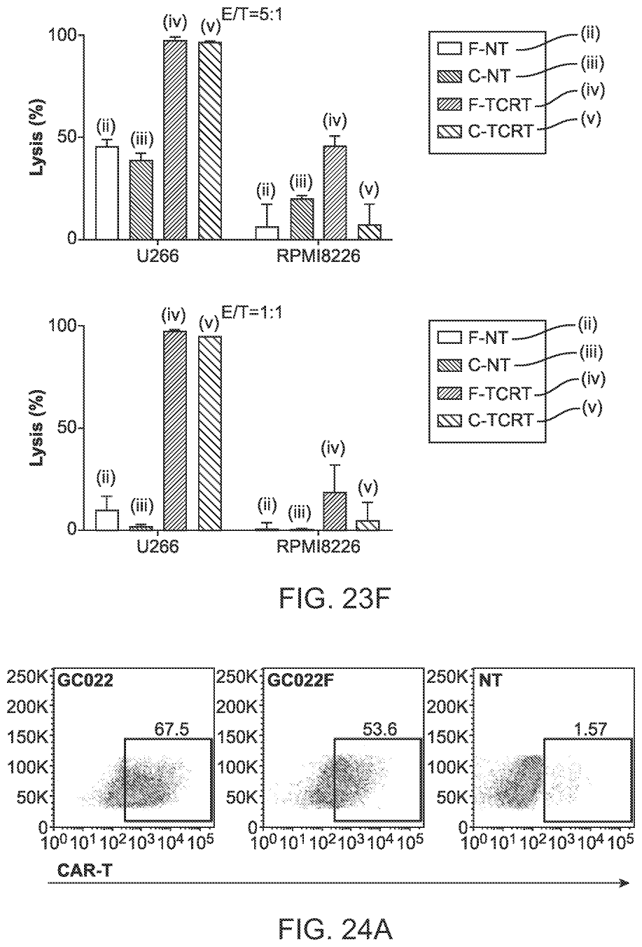

Nucleotide sequences encoding the engineered NY-ESO-1 TCR (e.g., NY-ESO-1 TCRT cDNA) were inserted into pCCL-cPPT Lentivirus plasmid. Subsequently, HEK293 cells were transfected with pCCL-cPPT and other packaging plasmids (helper plasmids). Following, e.g., 3 days after transfection, lentiviral (LV) particles were harvested and concentrated via centrifugation. T cells comprising the NY-ESO-1 TCRT gene were prepared following a procedure similar to that for F-CART cells, as provided in Examples 2 and 3 of the present disclosure. In such procedure, T cells were transfected with NY-ESO01 TCR LV particles for 1 day. After the transfection, without further expansion, the CAR-T cells of the present disclosure were obtained (also named FTCRT, F-TCRT, or F-TCR-T cells herein). In this procedure, the engineered T cells were not activated before transfection. An interaction between (i) T cells modified to express an engineered NY-ESO-1 TCR and (ii) cancer cells expressing a NY-ESO-1 peptide via HLA-A*02 is schematically illustrated in FIG. 23A.

Control cells comprising the NY-ESO-1 TCRT gene were prepared by transfecting T cells with NY-ESO-1 TCR LV particles using conventional methods, e.g., the methods disclosed in Example 4 of the present disclosure to prepare C-CART cells. After the activation and transfection (e.g., over the course of 1 day), the modified T cells were cultured for 8 days for expansion, to obtain the control cells (also named as the second reference cells, CTCRT, C-TCRT, or C-CAR-T cells).

### Analysis of TCRT Proliferation

A proliferative capacity (e.g., in vitro proliferation) of FTCRT cells and CTCRT cells were analyzed, and the results are shown in FIG. 23B. Briefly, two days after thawing frozen TCRT cells (e.g., FTCRT cells and CTRCT cells), the engineered T cells were stimulated with irradiated U266 twice a week, and the number of NY-ESO-1 TCRT cells were quantified by flow cytometry. As illustrated in FIG. 23B, FTCRT cells exhibited a higher proliferative capacity than the CTCRT cells control. A fold change in the number of FTCRT cells was at least about 5 times higher than a fold change in the number of CTCRT on day 5. A fold change in the number of FTCRT cells was at least about 3 times higher than a fold change in the number of CTCRT on day 8. A fold change in the number of FTCRT cells was at least about 6 to 7 times higher than a fold change in the number of CTCRT on day 12.

### Analysis of Lymphocyte Subpopulations

Lymphocyte subpopulations were analyzed in stimulated FTCRT cells an CTCRT cells using methods as illustrated in Examples 6 of the present disclosure. Briefly, thawed TCRT cells were stimulated with irradiated U266 for 3 days, and phenotype of TCRT cell in the FTCRT cells and the CTCRT cells were analyzed by conventional flow cytometry. The results are shown in FIG. 23C, in which naïve T cells with great differentiation potential can be indicated by being CD45RO⁻/CD62L⁺ (top plots) or CD45RA⁺/CCR7⁺ (bottom plots). The results suggest that the FTCRT cells exhibited a “younger” phenotype than the CTCRT cells, indicated by a higher percentage of native T cells. A proportion of CD45RO⁻/CD62L⁺ naïve T cells in FTCRT cells (69.2%) was about twice as high as that in CTCRT cells (34.6%). A proportion of CD45RA⁺/CCR7⁺ naïve T cells in FTCRT cells (29.3%) was about 3.7 times higher than that in CTCRT cells (7.87%).

Separately, T cell exhaustion was analyzed in stimulated FTCRT cells an CTCRT cells using methods as illustrated in Examples 14 of the present disclosure. Briefly, thawed TCRT cells were stimulated with irradiated U266 for 3 days, and phenotype of TCRT cell in the FTCRT cells and the CTCRT cells were analyzed by conventional flow cytometry. The results are shown in FIG. 23D, in which exhausted T cells are indicated by being PD1⁺/LAG3⁺ (top plots) or PD1⁺/TIM3⁺ (bottom plots). The results suggest that the FTCRT cells exhibited a less exhaustion than the CTCRT cells, indicated by a lower percentage of exhausted TCRT cells. A proportion of PD1⁺/LAG3⁺ T cells in FTCRT cells was non-detectable (0%), while that in CTCRT cells was significantly higher (4.65%). A proportion of PD1⁺/TIM3⁺ T cells in FTCRT cells (0.19%) was about 177 times lower than that in CTCRT cells (33.7%).

### In Vitro Tumor Killing Efficacy of TCRT Cells

Cytotoxicity of FTCRT cells against target cells (e.g., MCF-7 breast cancer cells presenting at least a fragment of the NY-ESO-1 protein) and that of CTCRT cells were compared using the real time cell analyzer (RTCA) assay as previously described in Example 17 of the present disclosure, using an effector to target ratio (i.e., E/T ratio) of 5:1 or 1:1. Briefly, thawed TCRT cells were stimulated with 2 rounds of irradiated U266. Following, TCRT cells were co-cultured with 2×10⁴ target cell in a E/T ratio 5:1 or 1:1. Controls included normal T cells (without the modified TCR against NY-ESO-1 fragment) subjected to either the FTCRT preparation procedure (as indicated by F-NT herein) or the conventional CTCRT preparation procedure (as indicated by C-NT herein). Target cell growth were monitored with RTCA, and the results are shown in FIG. 23E. The results indicate that the FTCRT cells exhibited enhanced cytotoxicity against MCF-7 cells at the E/T ratio of 1:1 (as indicated by a normalized cell index of MCF-7 of about 1.1 after 60 hours), as compared to the CTCRT cells (as indicated by a normalized cell index of MCF-7 of about 1.7 after 60 hours). Additionally, the results indicate that the FTCRT cells exhibited enhanced cytotoxicity against MCF-7 cells at the E/T ratio of 5:1 (as indicated by a normalized cell index of MCF-7 of about 0.5 after 60 hours), as compared to the CTCRT cells (as indicated by a normalized cell index of MCF-7 of about 0.7 after 60 hours).

### Cytotoxicity Luciferase Assay of TCRT Cells

Cytotoxicity of FTCRT cells against target cells (e.g., MCF-7 breast cancer cells presenting at least a fragment of the NY-ESO-1 protein) and that of CTCRT cells were compared using the luciferase assay as previously described in Example 21 of the present disclosure, using an effector to target ratio (i.e., E/T ratio) of 5:1 or 1:1. Briefly, thawed TCRT cells were stimulated with 2 rounds of irradiated U266 human B lymphocytes, or stimulated RPMI 8226 human B lymphocytes. Following, the TCRT cells were co-cultured with 2×10⁴ target cells in a E/T ratio 5:1 or 1:1. After 20 hours of co-culture, Luciferase substance were added in the co-culture system, and residual target cell were quantified based on luciferase activity. The results are shown in FIG. 23F. When stimulated with U266, FTCRT cells and CTCRT cells exhibited comparable cytotoxicity against the target cells at E/T ratio of 5:1 (top plot) or 1:1 (bottom plot). When stimulated with RPMI 8226, FTCRT cells exhibited enhanced lysis of target cells at E/T ratio of 5:1 (about 50%, top plot) as compared to that of CTCRT cells (less than about 10%, bottom plot). Additionally, when stimulated with RPMI 8226, FTCRT cells exhibited enhanced lysis of target cells at E/T ratio of 1:1 (about 20%, top plot) as compared to that of CTCRT cells (less than about 10%, bottom plot).

Overall, FTCRT cells configured to express the engineered TCR against a fragment of NY-ESO-1 protein exhibited (i) enhanced proliferative capacity, (ii) a higher proportion of naive T cells having greater memory and/or stemness, (iii) less cell exhaustion, and (iv) enhanced cytotoxicity against certain target cells as compared to CTCRT cells configured to express the same engineered TCR. Table 11 below shows a summary of the various in vitro findings of comparative studies between FTCRT cells and CTCRT cells.

TABLE 11
Summary of FTCRT vs. CTCRT.

| Performance Feature | FTCRT | CTCRT |
|---|---|---|
| Proliferation | +++ | + |
| Memory/Stemness | +++ | + |
| Exhaustion | + | +++ |
| Killing Capacity | +++ | +++ |
| (in vitro, U266) | | |
| Killing Capacity | ++ | + |
| (in vitro, MCF-7) | | |

### Example 25: In Vitro and In Vivo Analyses of F-CART Vs C-CART Subject Samples

The CART cells expressing a dual anti-CD19 and anti-CD22 CAR were prepared using the F-CART method and conventional method as provided in the previous Examples of the present disclosure, e.g., Example 24. Methods of subjecting T cells from the GC022 patient sample (e.g., as disclosed in Table 8 of the present disclosure) to the F-CART production processes, or products thereof, are denoted herein as GC022F. Methods of subjecting T cells from the GC022 patient sample to the conventional C-CART production processes, or products thereof, are denoted herein as GC022. Unlike conventional production process (e.g., C-CART) that is used to produce the GC022 C-CART cells, which requires 8-14 days of culture (e.g., 9 days), GC022F can produce and prepare CAR-T cells in one day, which can be provided to patients faster and also reduce the cost of production. In order to verify the safety and effectiveness of GC022F products, in vitro and in vivo experiments were conducted, as discussed below.

### Production of GC022F (F-CART) and GC022 (C-CART) Cells

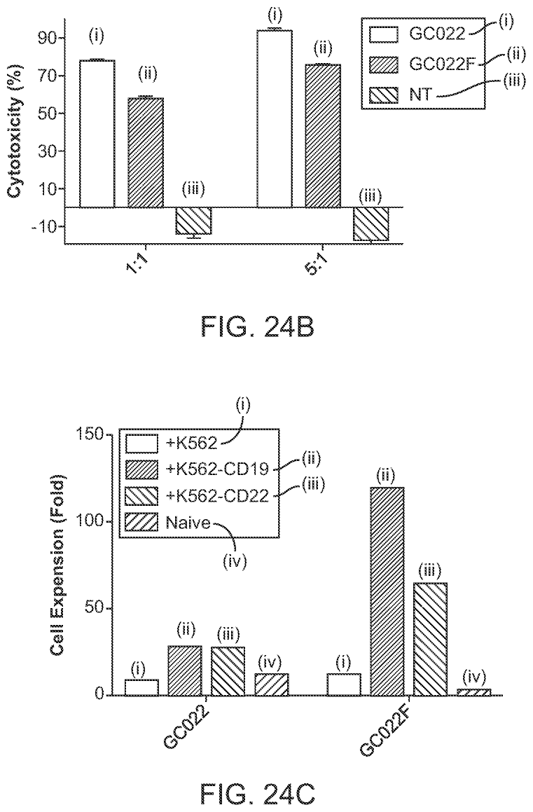

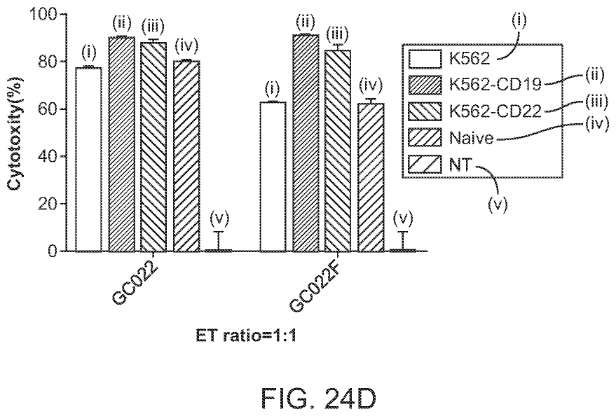

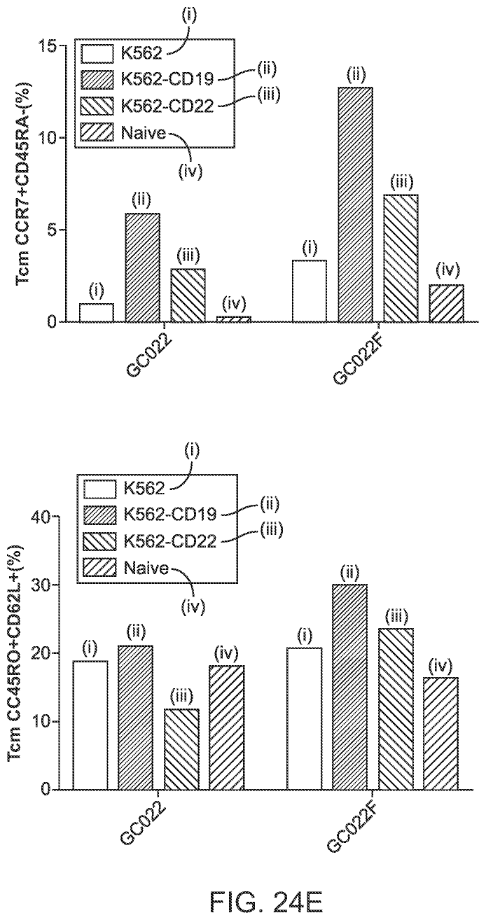

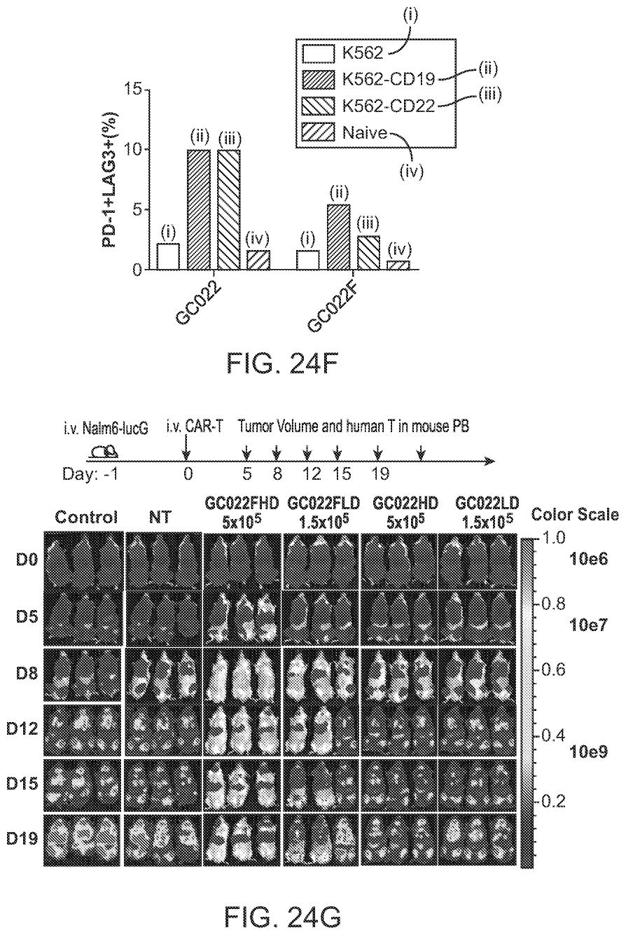

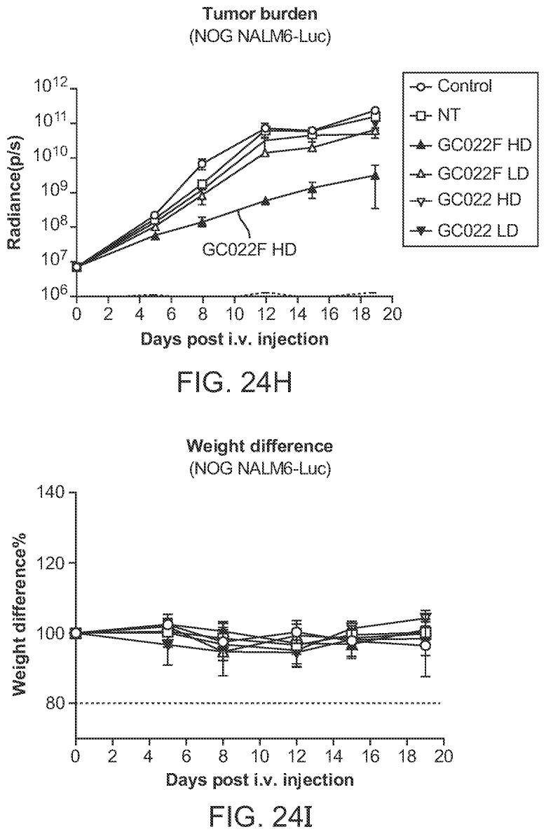

T cells from the B-ALL GC022 subject were thawed and treated (e.g., transduced) accordingly to produce the GC022F CART cells and the conventional GC022 CART cells. After 2 days of culture, flow cytometry analysis showed that more than 50% of both GC022F CART cells (53.6%) and the conventional GC022 CART cells (67.5%) expressed the CAR of interest. NT is a T cell control not transduced with GC022 retrovirus. The results are as shown in FIG. 24A.

### Cytotoxicity Luciferase Assay

Cytotoxicity of subject's GC022F CART and conventional GC022 CART was assessed as previously described, e.g., in Example 21 using an effector to target ratio (i.e., E/T ratio) of 1:1 or 5:1. Results are shown in FIG. 24B. The GC022F CART cells and the conventional GC022 CART cells were mixed with Raji-Luc cells as target cells, and incubated for a total of 20 hours. Substrates were added to determine the amount of Luciferase released in the cell culture solution, and the specific killing ratio was calculated. The results in FIG. 24B show that both the GC022F CART cells and the conventional GC022 CART cells exhibit comparable cytotoxicity against the target cells at the E/T ratio of 1:1 and 5:1. The control NT cells exhibited no cytotoxic activity under the same conditions.

### Cellular Expansion

Frozen samples of the GC022F CART cells and the conventional GC022 CART cells were thawed and cultured for 2 days. Subsequently, K562, K562-CD19, or K562-CD22 cells, each of which were inactivated by irradiation, were added to each CART cell culture system. A number of the K562, K562-CD19, or K562-CD22 cells added was twice that of the GC022F CART cells or the conventional GC022 CART cells. Control (Naïve) CART cells were not co-cultured with any one of the K562, K562-CD19, or K562-CD22 cells. On day 5, cells were counted, and passaged with the same stimulation. On day 8, cells were counted. As shown in FIG. 24C, control CART cells and CART cells co-cultured/stimulated with K562 cells that did not express CD19 and CD22 expanded (or proliferated) slowly. When stimulated with K562 cells expressing CD19 or CD22, both the GC022F CART cells and the conventional GC022 CART cells expanded (or proliferated) in large numbers, and the GC022F CART cells exhibited a greater expansion capacity than the conventional GC022 CART cells. The conventional GC022 CART cells and the GC0022F CART cells exhibited about a 26.4-fold and about a 118.5-fold expansion, respectively, under K562-CD19 stimulation, thus expansion of the GC022F CART cells was about 4.5 times greater than that of the conventional GC022 CART cells under CD19 stimulation. The conventional GC022 CART cells and the GC022F CART cells exhibited about a 26.7-fold and about a 63.4-fold expansion, respectively, under K562-CD22 stimulation, thus expansion of the GC022F CART cells was about 2.3 times greater than that of the conventional GC022 CART cells under CD22 stimulation. Overall, the GC022F CART cells showed enhanced expansion/proliferation capacity under antigen-specific stimulation in comparison to the conventional GC022 CART cells.

### Cytotoxicity Assay Following Antigen-Stimulated Cellular Expansion

In order to test whether CAR-T cells can maintain cytotoxicity against target cells (e.g., tumor killing function) after stimulation and expansion via antigen (e.g., CD19 or CD22), the GC022F CART cells and the conventional GC022 CART cells were antigen-stimulated and expanded (as shown in FIG. 24C), then co-cultured with Raji-Luc cells at a 1:1 E/T ratio for 20 hours. Afterwards, cytotoxicity of the CART cells assessed as previously described, e.g., in Example 21 using the Luciferase-based assay. As shown in FIG. 24D, both the GC022F CART cells and the conventional GC022 CART cells exhibited cytotoxicity against the Raji target cells after in vitro culture for antigen-stimulation and expansion. In some cases, CD19 or CD22 antigen-specific stimulation enhanced cytotoxicity of the GC022F CART cells and the conventional GC022 CART cells against the Raji target cells.

### Phenotype and Exhaustion

Lymphocyte subpopulations of the GC022F CART cells and the conventional GC022 CART cells were analyzed by conventional flow cytometry. Expression of markers (e.g., CCR7, CD45RA, CD45RO, CD62L, PD-1, and LAG3) were analyzed through flow cytometry. Subsequent to antigen-specific stimulation, as described above (e.g., 3 days of antigen-specific stimulation), and further cell culture (e.g., 5 days of additional cell culture), the CART cells were subjected to FACS analysis. As shown in FIG. 24E, the proportion of Tcm cells (CCR7⁺/CD45RA⁻) increased after CD19 or CD22 stimulation for both GC022F CART cells and conventional GC022 CART cells. Additionally, subsequent to CD19 or CD22 stimulation, the proportion of the Tcm cells in the GC022F CART cells was about two times greater than that in the conventional GC022 CART cells.

As shown in FIG. 24F, CART cells were assessed for exhibiting T cell exhaustion markers, such as PD-1 and LAG3. The proportion of PD-1⁺/LAG3⁺ cells (indicative of exhausted T cells) in the GC022 CART cells was increased after being stimulated by the antigen CD19 or CD22. However, the proportion of the PD-1⁺/LAG3⁺ cells in the CD19 antigen-stimulated GC022F CART cells (about 5%) was about 50% of that in the CD19 antigen-stimulated conventional GC022 CART cells (about 10%). Additionally, the proportion of the PD-1⁺/LAG3⁺ cells in the CD22 antigen-stimulated GC022F CART cells (about 2-3%) was about 20-30% of that in the CD22 antigen-stimulated conventional GC022 CART cells (about 10%). The method of the present disclosure resulted in reducing exhaustion of the T cells during production of CART cells, in comparison to conventional CART cell production methods.

### In Vivo Analysis for Tumor Cytotoxicity

NOG mice (NOD.Cg-Prkdcscid Il2rgtm1Sug/JicTac) were engrafted with NALM-6-LucG cells. Briefly, 5×10⁵ NALM-6 cells were injected to each mouse through tail vein injection, and the fluorescence value was measured after 1 day of growth of the model tumor cells. Mice were grouped according to tumor growth and treated with PBS, control T cells, and the GC022F CART cells, and the conventional GC022 CART cells, respectively. T cells were administered at a dose of 1×10⁶ cells. The GC0022F CART cells were administered at a high dose (GC022FHD) of 5×10⁵ cells or at a low dose (GC022FLD) of 1.5×10⁵ cells. The conventional GC022 CART cells were administered at a high dose (GC022HD) of 5×10⁵ cells or at a low dose (GC022LD) of 1.5×10⁵ cells. Luciferase measurements were performed twice a week (e.g., at day 0, day 5, day 8, day 12, day 15, and day 19) after the respective CART cell therapy to assess their effects, as shown in FIG. 24G. The results showed the GC022F CART cells exhibited enhanced tumor suppression and/or removal than the conventional GC022 CART cells from day 8. While the conventional GC022 CART cells induced a reduction in tumor cells by day 8 and an increase in the presence of tumor cells up to day 19, the GC022F CART cells induced removal of the tumor cells by day 8 and maintained tumor suppression up to day 19. A graphical summary of the bioluminescence imaging in FIG. 24G is shown in FIG. 24H.

FIG. 24I shows change in body weight of the mice throughout the abovementioned in vivo analysis. The results indicated that there were no detectable side effects such as weight loss up to day 19, suggesting that the GC0022F CART cell therapy may be safe and effective to treat or reduce tumor in a subject, and that the GC022F CART cell therapy of the present disclosure may be more therapeutically effective and cost-effective than any conventional GC022 CART cell therapy.

1.-22. (canceled)

23. A method of producing a population of engineered immune cells expressing a chimeric antigen receptor (CAR) and/or an engineered T cell receptor (TCR), comprising:
(a) contacting a population of immune cells obtained from a subject with an activation moiety (“population of contacted immune cells”);
(b) introducing a polynucleotide encoding the CAR or TCR in the population of contacted immune cells in (a), thereby producing a population of engineered immune cells expressing the CAR or TCR, and
(c) harvesting the population of engineered immune cells expressing the CAR or TCR for infusion to the subject;
wherein the total duration of both (a) and (b) is no more than 36 hours and wherein the method does not comprise an ex vivo expansion except (a) to (c).

24. The method of claim 23, wherein the harvesting comprises cryopreserving the population of engineered immune cells.

25. The method of claim 23, wherein the activation moiety is removed after step (b).

26. The method of claim 23, wherein the introducing in (b) is performed on the same day as the contacting in (a).

27. The method of claim 26, wherein the introducing in (b) is performed about 4 hours or less after the contacting in (a).

28. The method of claim 23, wherein the activation moiety and the polynucleotide together are in contact with the population of immune cells in (b) for no more than 24 hours.

29. The method of claim 23, wherein the population of immune cells are activated after (a).

30. The method of claim 28, wherein the activation moiety comprises anti-CD3 antibody and/or anti-CD28 antibody.

31. The method of claim 23, wherein the activation moiety is associated with the population of immune cells during the introducing in (b).

32. The method of claim 31, wherein the activation moiety is conjugated to a bead, a plate, and/or a matrix.

33. The method of claim 23, wherein the introducing in (b) comprises transducing the population of immune cells with a viral vector and/or a transposon vector.

34. The method of claim 33, wherein the viral vector is a retroviral vector, a lentiviral vector and/or an adeno-associated viral vector.

35. The method of claim 23, wherein the population of immune cells is seeded at a density from about 10⁴ cells/mL to about 10⁸ cells/mL.

36. The method of claim 33, wherein the viral vector is plated at a mean of infectivity (MOI) from about 0.1 to about 20.

37. The method of claim 23, further comprising contacting a cytokine to the population of immune cells.

38. The method of claim 37, wherein the cytokine comprises IL2, IL7, IL15 and/or IL21.

39. The method of claim 23, wherein the population of engineered immune cells are not cultured in the presence of human serum.

40. The method of claim 23, wherein the population of engineered immune cells is characterized in that cell memory T cells (TCM) in the population are more abundant than effector memory T cells (TEM).

41. A population of engineered immune cells produced by the method of claim 23.

42. A point-of-care facility comprising a cell infusion equipment configured to: infuse the population of engineered immune cells of claim 41.

43. A method of treating a cancer in a subject in need thereof, comprising infusing the population of engineered immune cells of claim 41 in the subject.


SEQUENCE LISTING (16 sequences)

SEQ ID NO: 1 - DNA, 493 bp, synthetic construct
  misc_feature 1..493: note = Description of Artificial Sequence: Synthetic polynucleotide
  source 1..493: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 2 - skipped

SEQ ID NO: 3 - DNA, 834 bp, synthetic construct
  misc_feature 1..834: note = Description of Artificial Sequence: Synthetic polynucleotide
  source 1..834: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcagactacaaagacgatgacgacaag

SEQ ID NO: 4 - skipped

SEQ ID NO: 5 - DNA, 321 bp, synthetic construct
  misc_feature 1..321: note = Description of Artificial Sequence: Synthetic polynucleotide
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 6 - skipped

SEQ ID NO: 7 - DNA, 336 bp, synthetic construct
  misc_feature 1..336: note = Description of Artificial Sequence: Synthetic polynucleotide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - skipped

SEQ ID NO: 9 - DNA, 1494 bp, synthetic construct
  misc_feature 1..1494: note = Description of Artificial Sequence: Synthetic polynucleotide
  source 1..1494: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 10 - skipped

SEQ ID NO: 11 - DNA, 1824 bp, synthetic construct
  misc_feature 1..1824: note = Description of Artificial Sequence: Synthetic polynucleotide
  source 1..1824: mol_type = other DNA
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagccttggaagcggagagggcagaggaagtcttctaacatgcggtgacgtggaggagaatcccggccctatgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggctaa

SEQ ID NO: 12 - skipped

SEQ ID NO: 13 - protein, 607 aa, synthetic construct
  REGION 1..607: note = Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSLGSGEGRGSLLTCGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - skipped

SEQ ID NO: 15 - protein, 9 aa, synthetic construct
  REGION 1..9: note = Description of Artificial Sequence: Synthetic peptide
SLLMWITQC

SEQ ID NO: 16 - protein, 6 aa, synthetic construct
  REGION 1..6: note = Description of Artificial Sequence: Synthetic 6xHis tag
HHHHHH